(19) United States
(12) Patent Application Publication
(10) Pub. No.: US 2007/0149570 A1
(43) Pub. Date: Jun. 28, 2007

(54) Piperidine derivative and use thereof

(76) Inventors: Yoshinori Ikeura, Osaka (JP); Junya Shirai, Osaka (JP); Takeshi Yoshikawa, Osaka (JP); Nobuki Sakauchi, Osaka (JP)

Correspondence Address: WENDEROTH, LIND & PONACK, L.L.P., 2033 K STREET N. W., SUITE 800, WASHINGTON, DC, 20006-1021, US

(21) Appl. No.: 11/701,380

(22) Filed: Feb. 2, 2007

Related U.S. Application Data
(63) Continuation-in-part of application No. PCT/JP06/15899, filed on Aug. 4, 2006.

(30) Foreign Application Priority Data
Aug. 4, 2005 (JP) 227183/2005

(51) Int. Cl. C07D 211/56; A61K 31/445

(52) U.S. Cl. 514/317; 546/223

(57) ABSTRACT

The present invention relates to a compound represented by the formula: 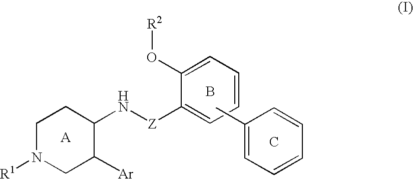
wherein Ar is a phenyl group optionally having substituent(s), R¹ is a hydrogen atom, a hydrocarbon group optionally having substituent(s), an acyl group or a heterocyclic group optionally having substituent(s), R² is a hydrogen atom, a C₁₋₆ alkyl group optionally having substituent(s) or a C₃₋₆ cycloalkyl group optionally having substituent(s), Z is a methylene group optionally having a C₁₋₆ alkyl group, ring A is a piperidine ring optionally further having substituent(s), ring B and ring C are benzene rings optionally further having substituent(s), and R² optionally form a ring together with the adjacent substituent on the ring B, except the compounds represented by the formula: 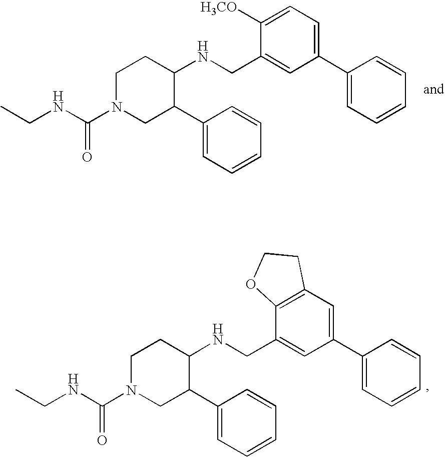 or a salt thereof. The compound of the present invention has a superior tachykinin receptor antagonistic action, particularly a substance P receptor antagonistic action, and is useful as a pharmaceutical agent, for example, tachykinin receptor antagonist, an agent for the prophylaxis or treatment of lower urinary tract symptoms, gastrointestinal diseases or central nerve diseases.

### TECHNICAL FIELD

The present invention relates to a novel piperidine derivative having excellent antagonistic action for a tachykinin receptor and use thereof.

### BACKGROUND ART

Tachykinin is a generic term for a group of neuropeptides. Substance P (SP), neurokinin A and neurokinin B are known in mammals, and these peptides are known to bind to the corresponding receptors (neurokinin-1, neurokinin-2 and neurokinin-3) that exist in a living body and thereby to exhibit various biological activities.

Of such neuropeptides, SP has the longest history and has been studied in detail. In 1931, the existence of SP in the extract from equine intestines was confirmed, and in 1971, its structure was determined. SP is a peptide consisting of 11 amino acids.

SP is broadly distributed over the central and peripheral nervous systems, and has various physiological activities such as vasodilation, enhancement of vascular extravasation, contraction of smooth muscles, excitation of neurons, salivation, enhancement of diuresis, immunological enhancement and the like, in addition to the function as a transmitter substance for primary sensory neurons. In particular, it is known that SP released from the terminal of the spinal (dorsal) horn due to a pain impulse transmits the information of pain to secondary neurons, and that SP released from the peripheral terminal induces an inflammatory response in the receptor thereof. Thus, it is considered that SP is involved in various disorders (e.g., pain, headache, particularly migraine, Alzheimer's disease, multiple sclerosis, cardiovascular modulation, chronic inflammatory diseases such as chronic rheumatic arthritis, respiratory diseases including asthma or allergic rhinitis, intestinal inflammatory diseases including ulcerative colitis and Crohn's disease, ocular damage and ocular inflammatory diseases, proliferative vitreous retinopathy, an irritable bowel syndrome, urinary frequency, psychosis, vomiting, etc.) [see, for example, Physiological Reviews, Vol. 73, pp. 229-308 (1993); Journal of Autonomic Pharmacology, Vol. 13, pp. 23-93 (1993)].

At present, the following compounds are known as those having antagonistic action for SP receptors.

EP-A-436,334 discloses a compound represented by the formula 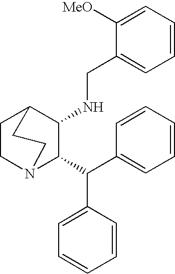
and the like,
WO92/17449 discloses a compound represented by the formula 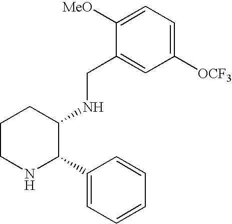
and the like,
WO95/16679 discloses a compound represented by the formula 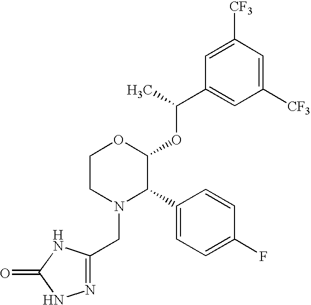
and the like, and
JP-A-9-263585 discloses a heterocyclic compound represented by the formula 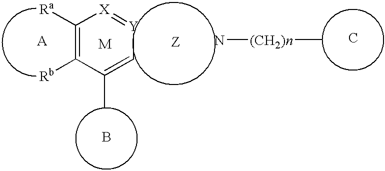
wherein Ring M is a heterocyclic ring wherein 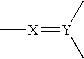
is one of —N═C<, —CO—N< or —CS—N<; R^{a} and R^{b} are bonded to each other to form Ring A, or they are the same or different and represent, independently, a hydrogen atom or a substituent on the Ring M; Ring A and Ring B represent, independently, an optionally substituted homocyclic or heterocyclic ring, with the proviso that at least one of them is an optionally substituted heterocyclic ring; Ring C is an optionally substituted homocyclic or heterocyclic ring; Ring Z is an optionally substituted nitrogen-containing heterocyclic ring; and n is an integer from 1 to 6, or a salt thereof and the like.

WO03/101964 describes a compound having a tachykinin receptor antagonistic action, which is represented by the formula 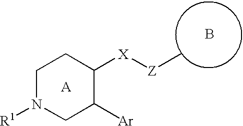
wherein Ar is an aryl group, an aralkyl group or an aromatic heterocyclic group, each of which may be substituted, R¹ is a hydrogen atom, an optionally substituted hydrocarbon group, an acyl group or an optionally substituted heterocyclic group, X is an oxygen atom or an optionally substituted imino group, Z is an optionally substituted methylene group, Ring A is a further optionally substituted piperidine ring, and Ring B is an optionally substituted aromatic ring, provided that when Z is a methylene group substituted with an oxo group, R¹ is not a methyl group, and when Z is a methylene group substituted with a methyl group, Ring B is a substituted aromatic ring, or a salt thereof.

### DISCLOSURE OF THE INVENTION

An object of the present invention is to provide a piperidine derivative having antagonistic action for a tachykinin receptor, etc. with a different chemical structure from the known compounds including the above-mentioned compounds, an agent for the prophylaxis or treatment of an abnormality of lower urinary tract functions comprising the derivative, and the like.

The present inventors have made extensive studies in consideration of the above-mentioned situation and, as a result, have found unexpectedly that piperidine derivatives represented by the formula (I) below or a salt thereof have excellent antagonistic action for a tachykinin receptor (particularly antagonistic action for a SP receptor) as based on their peculiar chemical structures and are sufficiently satisfactory as pharmaceutical agents. On the basis of these findings, the present inventors have completed the present invention.

Specifically, the present invention provides the following:
[1] a compound represented by the formula: 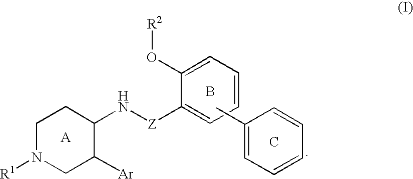
wherein Ar is a phenyl group optionally having substituent(s), R¹ is a hydrogen atom, a hydrocarbon group optionally having substituent(s), an acyl group or a heterocyclic group optionally having substituent(s), R² is a hydrogen atom, a C₁₋₆ alkyl group optionally having substituent(s) or a C₃₋₆ cycloalkyl group optionally having substituent(s), Z is a methylene group optionally having a C₁₋₆ alkyl group, ring A is a piperidine ring optionally further having substituent(s), ring B and ring C are benzene rings optionally further having substituent(s), and R² optionally forms a ring together with the adjacent substituent on the ring B, except the compounds represented by the formula: 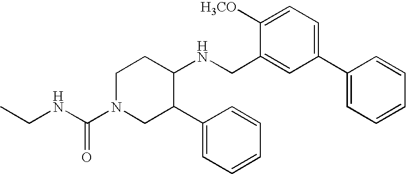
and the formula: 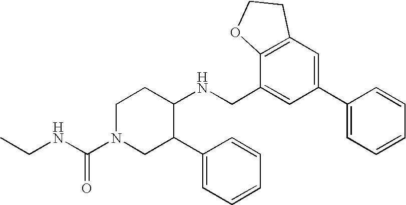
(hereinafter sometimes to be abbreviated as compound (I)), or a salt thereof;
[2] the compound of [1], which is represented by the formula: 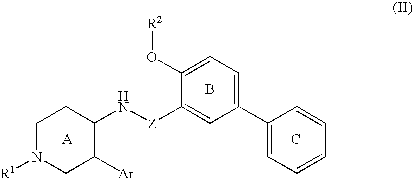
wherein the symbols in the formula are as defined in [1];
[3] the compound of [1], which is represented by the formula: 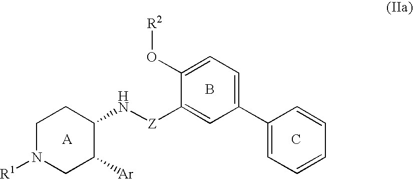
wherein the symbols in the formula are as defined in [1];
[4] the compound of any of [1]-[3], wherein R¹ is a hydrogen atom or an acyl group;
[5] the compound of any of [1]-[3], wherein R² is a hydrogen atom or a C₁₋₆ alkyl group optionally having substituent(s);
[6] the compound of any of [1]-[3], wherein Z is a methylene group optionally having a methyl group;
[7] the compound of [3], wherein Ar is a phenyl group optionally having 1 to 3 halogen atoms;
R¹ is (1) a hydrogen atom,
(2) a C₁₋₆ alkyl-carbonyl optionally having 1 or 2 substituents selected from the group consisting of (i) an amino, (ii) a C₁₋₆ alkoxy, (iii) a C₁₋₆ alkyl-carbonylamino, (iv) a C₁₋₆ alkoxy-carbonylamino, (v) a of alkylsulfonylamino, (vi) a 5- or 6-membered nitrogen-containing heterocyclic group optionally having 1 to 5 substituents selected from the group consisting of a C₁₋₆ alkyl and an oxo, said heterocyclic group optionally forms a spiro ring together with cyclopentane or cyclohexane, (vii) a C₁₋₆ alkyl-carbonyloxy, (viii) a hydroxy and (ix) a carbamoyl,
(3) a C₁₋₆ alkoxy-carbonyl,
(4) a C₁₋₆ alkylamino-carbonyl,
(5) a C₁₋₆ alkylsulfonyl,
(6) an aminocarbonylcarbonyl,
(7) a C₁₋₆ alkylamino-carbonylcarbonyl,
(8) a di-C₁₋₆ alkylamino-carbonylcarbonyl, or
(9) a piperidin-4-ylcarbonyl optionally having 1 or 2 substituents selected from the group consisting of (i) a C₁₋₆ alkyl-carbonyl optionally having a 5- or 6-membered nitrogen-containing heterocyclic group optionally having 1 or 2 oxo, (ii) a C₁₋₆ alkoxy-carbonyl, (iii) a C₁₋₆ alkylsulfonyl, (iv) a C₁₋₆ alkyl-carbonylamino-C₁₋₆ alkyl-carbonyl, (v) a di-C₁₋₆ alkyl-carbamoyl and (vi) an oxo;
R² is (1) a hydrogen atom or (2) a C₁₋₆ alkyl group optionally having 1 to 3 halogen atoms;
Z is a methylene group optionally having a methyl group;
ring A is a piperidine ring without a further substituent;
ring B is a benzene ring optionally further having a halogen, atom or a C₁₋₆ alkyl or ring B forms a 2,3-dihydrobenzofuran ring together with R²; and
ring C is a benzene ring optionally having 1 or 2 substituents selected from the group consisting of
(1) a cyano,
(2) a nitro,
(3) a halogen atom,
(4) a C₁₋₆ alkyl optionally having 1 to 3 halogen atoms,
(5) a C₁₋₆ alkynyl,
(6) a C₁₋₆ alkoxy optionally having 1 to 3 halogen atoms,
(7) a C₁₋₆ alkylthio,
(8) a C₁₋₆ alkylsulfonyl,
(9) a di-C₁₋₆ alkylamino,
(10) a C₁₋₆ alkyl-carbonyl,
(11) a C₁₋₆ alkyl-carbonylamino,
(12) a C₁₋₆ alkoxy-carbonyl and
(13) a carbamoyl;
[8] a compound represented by the formula: 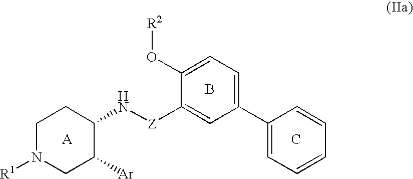
wherein
Ar is a phenyl group optionally having 1 to 3 halogen atoms;
R¹ is (1) a hydrogen atom,
(2) a C₁₋₆ alkyl-carbonyl optionally having 1 or 2 substituents selected from the group consisting of (i) an amino, (ii) a C₁₋₆ alkoxy, (iii) a C₁₋₆ alkyl-carbonylamino, (iv) a C₁₋₆ alkoxy-carbonylamino, (v) a C₁₋₆ alkylsulfonylamino, (vi) a 5- or 6-membered nitrogen-containing heterocyclic group optionally having 1 to 5 substituents selected from the group consisting of a C₁₋₆ alkyl and an oxo, said heterocyclic group optionally forms a spiro ring together with cyclopentane or cyclohexane, (vii) a C₁₋₆ alkyl-carbonyloxy, (viii) a hydroxy and (ix) a carbamoyl,
(3) a C₁₋₆ alkoxy-carbonyl,
(4) a C₁₋₆ alkylsulfonyl,
(5) an aminocarbonylcarbonyl,
(6) a C₁₋₆ alkylamino-carbonylcarbonyl,
(7) a di-C₁₋₆ alkylamino-carbonylcarbonyl, or
(8) a piperidin-4-ylcarbonyl optionally having 1 or 2 substituents selected from the group consisting of (i) a C₁₋₆ alkyl-carbonyl optionally having a 5- or 6-membered nitrogen-containing heterocyclic group optionally having 1 or 2 oxo, (ii) a C₁₋₆ alkoxy-carbonyl, (iii) a C₁₋₆ alkylsulfonyl, (iv) a C₁₋₆ alkyl-carbonylamino-C₁₋₆ alkyl-carbonyl, (v) a di-C₁₋₆ alkyl-carbamoyl and (vi) an oxo;
R² is (1) a hydrogen atom or (2) a C₁₋₆ alkyl group optionally having 1 to 3 halogen atoms;
Z is a methylene group optionally having a methyl group;
ring A is a piperidine ring without a further substituent;
ring B is a benzene ring optionally further having a halogen atom or a C₁₋₆ alkyl or ring B forms a 2,3-dihydrobenzofuran ring together with R²; and
ring C is a benzene ring optionally having 1 or 2 substituents selected from the group consisting of
(1) a cyano,
(2) a nitro,
(3) a halogen atom,
(4) a C₁₋₆ alkyl optionally having 1 to 3 halogen atoms,
(5) a C₁₋₆ alkynyl,
(6) a C₁₋₆ alkoxy optionally having 1 to 3 halogen atoms,
(7) a C₁₋₆ alkylthio,
(8) a C₁₋₆ alkylsulfonyl,
(9) a di-C₁₋₆ alkylamino,
(10) a C₁₋₆ alkyl-carbonyl,
(11) a C₁₋₆ alkyl-carbonylamino,
(12) a C₁6 alkoxy-carbonyl and
(13) a carbamoyl,
   or a salt thereof;
[9]N-{2-[(3R,4S)-4-({[4′-chloro-4-(trifluoromethoxy)biphenyl-3-yl]methyl}amino)-3-phenylpiperidin-1-yl]-2-oxoethyl}acetamide,
3′-[({(3R,4S)-1-[(5,5-dimethyl-2,4-dioxo-1,3-oxazolidin-3-yl)acetyl]-3-phenylpiperidin-4-yl}amino)methyl]-2-fluoro-4′-(trifluoromethoxy)biphenyl-4-carbonitrile,
2-fluoro-3′-({[(3R,4S)-1-glycoloyl-3-phenylpiperidin-4-yl]amino}methyl)-4′-(trifluoromethoxy)biphenyl-4-carbonitrile,
3′-[({(3R,4S)-1-[(1-acetylpiperidin-4-yl)carbonyl]-3-phenylpiperidin-4-yl}amino)methyl]-2-fluoro-4′-(trifluoromethoxy)biphenyl-4-carbonitrile,
3′-[({(3R,4S)-1-[(2,6-dioxopiperidin-4-yl)carbonyl]-3-phenylpiperidin-4-yl}amino)methyl]-2-fluoro-4′-(trifluoromethoxy)biphenyl-4-carbonitrile,
2-[(3R,4S)-4-({[4′-cyano-2′-fluoro-4-(trifluoromethoxy)biphenyl-3-yl]methyl}amino)-3-phenylpiperidin-1-yl]-2-oxoacetamide,
3-{2-[(3R,4S)-4-({[4′-chloro-4-(trifluoromethoxy)biphenyl-3-yl]methyl}amino)-3-phenylpiperidin-1-yl]-2-oxoethyl}-5,5-dimethyl-1,3-oxazolidine-2,4-dione,
4-{[(3R,4S)-4-({[4′-chloro-2′-fluoro-4-(trifluoromethoxy)biphenyl-3-yl]methyl}amino)-3-phenylpiperidin-1-yl]carbonyl}piperidine-2,6-dione,
3′-[({(3R,4S)-1-[(5,5-dimethyl-2,4-dioxo-1,3-oxazolidin-3-yl)acetyl]-3-phenylpiperidin-4-yl}amino)methyl]-4′-(trifluoromethoxy)biphenyl-4-carbonitrile,
   or a salt thereof;
[10] a prodrug of the compound of [1];
[11] a pharmaceutical agent comprising the compound of [1] or a prodrug thereof;
[12] the pharmaceutical agent of [11], which is a tachykinin receptor antagonist;
[13] the pharmaceutical agent of [11], which is an agent for the prophylaxis or treatment of lower urinary tract symptoms, a digestive organ disease or a central nerve disease;
[14] the pharmaceutical agent of [11], which is an agent for the prophylaxis or treatment of overactive bladder, lower urinary tract symptoms associated with benign prostatic hyperplasia, pelvic visceral pain, lower urinary tract symptoms associated with chronic prostatitis, lower urinary tract symptoms associated with interstitial cystitis, irritable bowel syndrome, inflammatory bowel disease, vomiting, nausea, depression, anxiety neurosis, anxiety or sleep disorder (insomnia);
[15] a method for the prophylaxis or treatment of lower urinary tract symptoms, a digestive organ disease or a central nerve disease, which comprises administering an effective amount of the compound of [1] or a prodrug thereof to a mammal;
[16] use of the compound of [1] or a prodrug thereof for the production of an agent for the prophylaxis or treatment of lower urinary tract symptoms, a digestive organ disease or a central nerve disease; and the like.

Compound (I) of the present invention and a salt thereof and a prodrug thereof have a high antagonistic action for a tachykinin receptor, particularly an antagonistic action for substance P receptor, and have low toxicity, and are safe as pharmaceutical agents. Therefore, compound (I) of the present invention and a salt thereof and a prodrug thereof are useful as medicaments, for example, a tachykinin receptor antagonist, an agent for the prophylaxis or treatment of an abnormality of lower urinary tract functions and the like.

### BEST MODE FOR EMBODYING THE INVENTION

Ar is a phenyl group optionally having substituent(s).

As the substituent of the “phenyl group”, for example, 1 to 3 substituents selected from the group consisting of (1) a halogen atom (e.g., fluorine, chlorine, bromine, iodine, etc.), (2) a C₁₋₃ alkylenedioxy (e.g., methylenedioxy, ethylenedioxy, etc.), (3), a nitro, (4) a cyano, (5) an optionally halogenated C₁₋₆ alkyl, (6) an optionally halogenated C₂₋₆ alkenyl, (7) an optionally halogenated C₂₋₆ alkynyl, (8) an optionally halogenated C₃₋₆ cycloalkyl, (9) a C₆₋₁₄ aryl (e.g., phenyl, 1-naphthyl, 2-naphthyl, biphenylyl, 2-anthryl, etc.), (10) an optionally halogenated C₁₋₆ alkoxy, (11) an optionally halogenated C₁₋₆ alkylthio or mercapto, (12) a hydroxy, (13) an amino, (14) a mono-C₁₋₆ alkylamino (e.g., methylamino, ethylamino, etc.), (15) a mono-C₆₋₁₄ arylamino (e.g., phenylamino, 1-naphthylamino, 2-naphthylamino, etc.), (16) a di-C₁₋₆ alkylamino (e.g., dimethylamino, diethylamino, etc.), (17) a di-C₆₋₁₄ arylamino (e.g., diphenylamino, etc.), (18) an acyl, (19) an acylamino, (20) an acyloxy, (21) a 5- to 7-membered cyclic amino optionally having substituent(s), (22) a 5- to 10-membered aromatic heterocyclic group (e.g., 2- or 3-thienyl, 2-, 3- or 4-pyridyl, 2-, 3-, 4-, 5- or 8-quinolyl, 1-, 3-, 4- or 5-isoquinolyl, 1-, 2- or 3-indolyl, 2-benzothiazolyl, 2-benzo[b]thienyl, benzo[b]furanyl, etc.), (23) a sulfo, (24) a C₆₋₁₄ aryloxy (e.g., phenyloxy, naphthyloxy, etc.), (25) a group which is a combination of 1 to 3 groups from the above-mentioned (1)-(24) and the like can be mentioned.

As the above-mentioned “optionally halogenated C₁₋₆ alkyl”, for example, a C₁₋₆ alkyl (e.g., methyl, ethyl, propyl, isopropyl, butyl, isobutyl, sec-butyl, tert-butyl, pentyl, hexyl, etc.) optionally having 1 to 5, preferably 1 to 3, halogen atoms (e.g., fluorine, chlorine, bromine, iodine, etc.) and the like can be mentioned. Specific examples include methyl, chloromethyl, difluoromethyl, trichloromethyl, trifluoromethyl, ethyl, 2-bromoethyl, 2,2,2-trifluoroethyl, pentafluoroethyl, propyl, 3,3,3-trifluoropropyl, isopropyl, butyl, 4,4,4-trifluorobutyl, isobutyl, sec-butyl, tert-butyl, pentyl, isopentyl, neopentyl, 5,5,5-trifluoropentyl, hexyl, 6,6,6-trifluorohexyl and the like.

As the above-mentioned “optionally halogenated C₂₋₆ alkenyl”, for example, a C₂₋₆ alkenyl (e.g., vinyl, allyl, isopropenyl, butenyl, isobutenyl, sec-butenyl, etc.) optionally having 1 to 5, preferably 1 to 3, halogen atoms (e.g., fluorine, chlorine, bromine, iodine, etc.) and the like can be mentioned. Specific examples include vinyl, allyl, isopropenyl, butenyl, isobutenyl, sec-butenyl, 3,3,3-trifluoro-1-propenyl, 4,4,4-trifluoro-1-butenyl and the like.

As the above-mentioned “optionally halogenated C₂₋₆ alkynyl”, for example, a C₂₋₆ alkynyl (e.g., ethynyl, propargyl, butynyl, 1-hexynyl, etc.) optionally having 1 to 5, preferably 1 to 3, halogen atoms (e.g., fluorine, chlorine, bromine, iodine, etc.) and the like can be mentioned. Specific examples include ethynyl, propargyl, butynyl, 1-hexynyl, 3,3,3-trifluoro-1-propynyl, 4,4,4-trifluoro-1-butynyl and the like.

As the above-mentioned “optionally halogenated C₃₋₆ cycloalkyl”, for example, a C₃₋₆ cycloalkyl (e.g., cyclopropyl, cyclobutyl, cyclopentyl, cyclohexyl, etc.) optionally having 1 to 5, preferably 1 to 3, halogen atoms (e.g., fluorine, chlorine, bromine, iodine, etc.) and the like can be mentioned. Specific examples include cyclopropyl, cyclobutyl, cyclopentyl, cyclohexyl, 4,4-dichlorocyclohexyl, 2,2,3,3-tetrafluorocyclopentyl, 4-chlorocyclohexyl and the like.

As the above-mentioned “optionally halogenated C₁₋₆ alkoxy”, for example, a C₁₋₆ alkoxy (e.g., methoxy, ethoxy, propoxy, isopropoxy, butoxy, isobutoxy, sec-butoxy, pentyloxy, hexyloxy, etc.) optionally having 1 to 5, preferably 1 to 3, halogen atoms (e.g., fluorine, chlorine, bromine, iodine, etc.) and the like can be mentioned. Specific examples include methoxy, difluoromethoxy, trifluoromethoxy, ethoxy, 2,2,2-trifluoroethoxy, propoxy, isopropoxy, butoxy, 4,4,4-trifluorobutoxy, isobutoxy, sec-butoxy, pentyloxy, hexyloxy and the like.

As the above-mentioned “optionally halogenated C₁₋₆ alkylthio”, for example, a C₁₋₆ alkylthio (e.g., methylthio, ethylthio, propylthio, isopropylthio, butylthio, sec-butylthio, tert-butylthio, etc.) optionally having 1 to 5, preferably 1 to 3, halogen atoms (e.g., fluorine, chlorine, bromine, iodine, etc.) and the like can be mentioned. Specific examples include methylthio, difluoromethylthio, trifluoromethylthio, ethylthio, propylthio, isopropylthio, butylthio, 4,4,4-trifluorobutylthio, pentylthio, hexylthio and the like.

As the above-mentioned “acyl”, for example, —(C═O)—R³, —(C═S)—R³, —SO₂—R³, —SO—R³, —(P═O)(OR⁴)(OR⁴′) (R³ is a hydrogen atom, a hydrocarbon group optionally having substituent(s), an amino group optionally having substituent(s), a hydroxy group optionally having a substituent or a heterocyclic group optionally having substituent(s), and R⁴ and R^{4′} are the same or different and each is a hydrogen atom or a hydrocarbon group optionally having substituent(s)) and the like can be mentioned.

The “hydrocarbon group optionally having substituent(s)” represented by R³, R⁴ and R^{4′} includes, for example, the same group as those referred to herein for the “hydrocarbon group optionally having substituent(s)” represented by R¹ which will be described below.

The “substituent” of the “amino group optionally having substituent(s)” represented by R³ includes, for example, a hydrocarbon group optionally having substituent(s), a heterocyclic group optionally having substituent(s), a hydroxy group optionally having a substituent, an acyl group and the like.

The “hydrocarbon group optionally having substituent(s)” as the “substituent” of the “amino group optionally having substituent(s)” represented by R³ includes, for example, the same group as those referred to herein for the “hydrocarbon group optionally having substituent(s)” represented by R¹ which will be described below.

The “heterocyclic group optionally having substituent(s)” as the “substituent” of the “amino group optionally having substituent(s)” represented by R³ includes, for example, the same group as those referred to herein for the “heterocyclic group optionally having substituent(s)” represented by R¹ which will be described below.

The “hydroxy group optionally having a substituent” as the “substituent” of the “amino group optionally having substituent(s)” represented by R³ includes, for example, (i) a hydroxy group, (ii) a C₁₋₆ alkoxy group (e.g., a methoxy group, an ethoxy group, a propoxy group, an isopropoxy group, a butoxy group, a tert-butoxy group, etc.), (iii) a C₆₋₁₄ aryloxy group (e.g., a phenyloxy group, a naphthyloxy group, etc.), (iv) a formyloxy group or a C₁₋₆ alkyl-carbonyloxy group (e.g., an acetoxy group, a propionyloxy group, etc.) and (v) a C₆₋₁₄ aryl-carbonyloxy group (e.g., a benzoyloxy group, a naphthylcarbonyloxy group, etc.) and the like, and preferably, a hydroxy group and a C₁₋₆ alkoxy group (e.g., a methoxy group, an ethoxy group, a propoxy group, an isopropoxy group, etc.).

The “acyl group” as the “substituent” of the “amino group optionally having substituent(s)” represented by R³ includes, for example, —(C═O)—R″, —(C═S)—R″, —SO₂—R″, —SO—R″, —(C═O)NR″R′″, —(C═O)O—R″, —(C═S)O—R″, —(C═S)NR″R′″ (R″ is a hydrogen atom or a hydrocarbon group optionally having substituent(s), R′″ is a hydrogen atom or a lower alkyl group (e.g., a C₁₋₆ alkyl group such as methyl, ethyl, propyl, isopropyl, butyl, isobutyl, sec-butyl, tert-butyl, pentyl, hexyl, etc., and particularly preferably a C₁₋₃ alkyl group such as methyl, ethyl, propyl, isopropyl, etc.) and the like.

The “hydrocarbon group optionally having substituent(s)” represented by R″ includes, for example, the same group as those referred to herein for the “hydrocarbon group optionally having substituent(s)” represented by R¹ which will be described below.

The “C₁₋₆ alkoxy group”, “C₆₋₁₄ aryloxy group”, “formyloxy group”, “C₁₋₆ alkyl-carbonyloxy group” and “C₆₋₁₄ aryl-carbonyloxy group” exemplified as the “hydroxy group optionally having a substituent” as the “substituent” of the “amino group optionally having substituent(s)” represented by R³, may be optionally further substituted with the same group as those referred to herein for the “substituent” of the “hydrocarbon group optionally having substituent(s)” represented by R¹ which will be described below and the like, and such substituent is preferably a halogen atom (e.g., fluorine, chlorine, bromine, etc.) and the like.

The “amino group optionally having substituent(s)” represented by R³ may form a cyclic amino group (e.g., a 5- to 9-membered cyclic amino group having 1 to 3 heteroatoms such as an oxygen atom, a sulfur atom, etc. in addition to a nitrogen atom (e.g., a pyrrolidino (1-pyrrolidinyl) group, a piperidino group, a piperazino (1-piperazinyl) group, a morpholino group, etc.) and the like.

The “hydroxy group optionally having a substituent” represented by R³ includes, for example, the same group as those referred to herein for the “hydroxy group optionally having a substituent” as the “substituent” of the “amino group optionally having substituent(s)” represented by R³ which is described above, and the like.

The “heterocyclic group optionally having substituent(s)” represented by R³ includes, for example, the same group as those referred to herein for the “heterocyclic group optionally having substituent(s)” represented by R¹ which will be described below.

As the above-mentioned “acylamino”, for example, formylamino, C₁₋₆ alkyl-carbonylamino (e.g., acetylamino, etc.), heterocyclyl-C₁₋₆ alkyl-carbonylamino (e.g., piperidino-acetylamino optionally having oxo, etc.), C₃₋₇ cycloalkyl-carbonylamino (e.g., cyclopropylcarbonylamino, etc.), C₆₋₁₄ aryl-carbonylamino (e.g., phenylcarbonylamino, naphthylcarbonylamino, etc.), heterocyclylcarbonylamino (e.g., thienylcarbonylamino, furylcarbonylamino, pyrrolylcarbonylamino, etc.), C₁₋₆ alkoxy-carbonylamino (e.g., methoxycarbonylamino, ethoxycarbonylamino, propoxycarbonylamino, butoxycarbonylamino, etc.), C₆₋₁₄ aryloxy-carbonylamino (e.g., phenoxycarbonylamino, naphthoxycarbonylamino, etc.), heterocyclyloxy-carbonylamino, C₁₋₆ alkylsulfonylamino (e.g., methylsulfonylamino, ethylsulfonylamino, etc.), C₆₋₁₄ arylsulfonylamino (e.g., phenylsulfonylamino, 2-naphthylsulfonylamino, 1-naphthylsulfonylamino, etc.), heterocyclylsulfonylamino, ureido, mono- or di-C₁₋₆ alkyl-ureido (e.g., methylureido, dimethylureido, etc.), mono- or di-C₆₋₁₄ aryl-ureido (e.g., phenylureido, diphenylureido, etc.) and the like can be mentioned.

As the above-mentioned “acyloxy”, for example, formyloxy, C₁₋₆ alkyl-carbonyloxy (e.g., acetoxy, propionyloxy, etc.), heterocyclyl-C₁₋₆ alkyl-carbonyloxy, C₃₋₇ cycloalkyl-carbonyloxy (e.g., cyclopropylcarbonyloxy, etc.), C₆₋₁₄ aryl-carbonyloxy (e.g., benzoyloxy, naphthylcarbonyloxy, etc.), heterocyclylcarbonyloxy (e.g., nicotinoyloxy, etc.), C₁₋₆ alkoxy-carbonyloxy (e.g., methoxycarbonyloxy, ethoxycarbonyloxy, propoxycarbonyloxy, butoxycarbonyloxy, etc.), C₆₋₁₄ aryloxy-carbonyloxy, heterocyclyloxy-carbonyloxy, mono-C₁₋₆ alkyl-carbamoyloxy (e.g., methylcarbamoyloxy, ethylcarbamoyloxy, etc.), di-C₁₋₆ alkyl-carbamoyloxy (e.g., dimethylcarbamoyloxy, diethylcarbamoyloxy, etc.), C₆₋₁₄ aryl-carbamoyloxy (e.g., phenylcarbamoyloxy, naphthylcarbamoyloxy, etc.) and the like can be mentioned.

Here, as the heterocyclic group of the heterocyclyl-C₁₋₆ alkyl-carbonylamino, heterocyclylcarbonylamino, heterocyclyloxy-carbonylamino, heterocyclylsulfonylamino, heterocyclyl-C₁₋₆ alkyl-carbonyloxy, heterocyclylcarbonyloxy and heterocyclyloxy-carbonyloxy, for example, a 5- to 14-membered (preferably 5- to 9-membered, more preferably 5- or 6-membered) non-aromatic heterocyclic group (e.g., pyrrolidinyl, tetrahydrofuryl, tetrahydrothienyl, piperidyl, tetrahydropyranyl, morpholinyl, thiomorpholinyl, piperazinyl) or aromatic heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl) containing, besides carbon atom, 1 to 4 heteroatoms of one or two kinds selected from the group consisting of a nitrogen atom, an oxygen atom and a sulfur atom, and optionally having 1 or 2 oxo and the like, and the like can be used.

As the “5- to 7-membered cyclic amino” of the above-mentioned “5- to 7-membered cyclic amino optionally having substituent(s)”, for example, a 5- to 7-membered saturated cyclic amino such as morpholino, thiomorpholino, piperazin-1-yl, piperidino, pyrrolidin-1-yl and the like can be mentioned. As the “substituent” of the “5- to 7-membered cyclic amino optionally having substituent(s)”, for example, 1 to 3 substituents selected from the group consisting of a C₁₋₆ alkyl (e.g., methyl, ethyl, propyl, isopropyl, butyl, isobutyl, sec-butyl, tert-butyl, pentyl, hexyl, etc.), a C₆₋₁₄ aryl (e.g., phenyl, 1-naphthyl, 2-naphthyl, biphenylyl, 2-anthryl, etc.), a 5- to 10-membered aromatic heterocyclic group (e.g., 2- or 3-thienyl, 2-, 3- or 4-pyridyl, 2-, 3-, 4-, 5- or 8-quinolyl, 1-, 3-, 4- or 5-isoquinolyl, 1-, 2- or 3-indolyl, 2-benzothiazolyl, 2-benzo[b]thienyl, benzo[b]furanyl, etc.) and the like can be mentioned.

As Ar, a phenyl group optionally having a halogen atom (e.g., fluorine atom) is preferable, and a phenyl group optionally substituted by a fluorine atom at the para-position and the like is more preferable. Particularly preferred is an unsubstituted phenyl group.

R¹ is a hydrogen atom, a hydrocarbon group optionally having substituent(s), an acyl group or a heterocyclic group optionally having substituent(s).

As the “hydrocarbon group” of the “hydrocarbon group optionally having substituent(s)” represented by R¹, for example, an aliphatic hydrocarbon group, a monocyclic saturated hydrocarbon group, an aromatic hydrocarbon group and the like can be mentioned, with preference given to such group having 1 to 16 carbon atoms. Specifically, for example, an alkyl group, an alkenyl group, an alkynyl group, a cycloalkyl group, an aryl group and the like are used.

As the “alkyl group”, for example, a lower alkyl group and the like are preferable and, for example, a C₁₋₆ alkyl group (e.g., methyl, ethyl, propyl, isopropyl, butyl, isobutyl, sec-butyl, tert-butyl, pentyl, hexyl, etc.) and the like are widely used.

As the “alkenyl group”, for example, a lower alkenyl group and the like are preferable and, for example, a C₂₋₆ alkenyl group (e.g., vinyl, 1-propenyl, allyl, isopropenyl, butenyl, isobutenyl, etc.) and the like are widely used.

As the “alkynyl group”, for example, a lower alkynyl group and the like are preferable and, for example, a C₂₋₆ alkynyl group (e.g., ethynyl, propargyl, 1-propynyl, etc.) and the like are widely used.

As the “cycloalkyl group”, for example, a lower cycloalkyl group and the like are preferable and, for example, a C₃₋₆ cycloalkyl group (e.g., cyclopropyl, cyclobutyl, cyclopentyl, cyclohexyl, etc.) and the like are widely used.

As the “aryl group”, for example, a C₆₋₁₄ aryl group (e.g., phenyl, 1-naphthyl, 2-naphthyl, biphenylyl, 2-anthryl, etc.) and the like are preferable and, for example, phenyl group and the like are widely used.

As the substituent that the “hydrocarbon group” of the “hydrocarbon group optionally having substituent(s)” represented by R¹ may have, for example, (1) a halogen atom (e.g., fluorine atom, chlorine atom, bromine atom, iodine atom, etc.), (2) a nitro group, (3) a cyano group, (4) a hydroxy group, (5) an optionally halogenated lower alkyl group (e.g., an optionally halogenated C₁₋₆ alkyl group such as methyl, chloromethyl, difluoromethyl, trichloromethyl, trifluoromethyl, ethyl, 2-bromoethyl, 2,2,2-trifluoroethyl, pentafluoroethyl, propyl, 3,3,3-trifluoropropyl, isopropyl, butyl, isobutyl, sec-butyl, tert-butyl, 4,4,4-trifluorobutyl, pentyl, isopentyl, neopentyl, 5,5,5-trifluoropentyl, hexyl, 6,6,6-trifluorohexyl and the like, etc.), (6) an optionally halogenated C₂₋₆ alkenyl, (7) an optionally halogenated C₂₋₆ alkynyl, (8) an optionally halogenated C₃₋₆ cycloalkyl, (9) an optionally halogenated lower alkoxy group (e.g., a C₁₋₆ alkoxy group such as methoxy, ethoxy, propoxy, isopropoxy, butoxy, isobutoxy, pentyloxy, hexyloxy and the like, etc.), (10) an acyloxy, (11) an optionally halogenated C₁₋₆ alkylthio or mercapto, (12) an acyl, (13) an amino group, (14) a mono-lower alkylamino group (e.g., a mono-C₁₋₆ alkylamino group such as methylamino, ethylamino and the like, etc.), (15) a di-lower alkylamino group (e.g., a di-C₁₋₆ alkylamino group such as dimethylamino, diethylamino and the like, etc.), (16) a mono-C₆₋₁₄ arylamino (e.g., phenylamino, 1-naphthylamino, 2-naphthylamino, etc.), (17) a di-C₆₋₁₄ arylamino (e.g., diphenylamino, etc.), (18) an acylamino, (19) a carboxyl group, (20) an aryl group (e.g., a C₆₋₁₄ aryl group such as phenyl, naphthyl, biphenylyl, 2-anthryl and the like, etc.), (21) an aryloxy group (e.g., a C₆₋₁₄ aryloxy group such as phenyloxy, naphthyloxy and the like, etc.), (22) an optionally halogenated lower alkylcarbonylamino group (e.g., an optionally halogenated C₁₋₆ alkyl-carbonylamino group such as acetylamino, trifluoroacetylamino and the like, etc.), (23) an optionally halogenated lower alkylsulfonylamino group (e.g., an optionally halogenated C₁₋₆ alkylsulfonylamino group such as methylsulfonylamino, trifluoromethylsulfonylamino and the like, etc.), (24) an optionally halogenated lower alkoxycarbonylamino group (e.g., an optionally halogenated C₁₋₆ alkoxy-carbonylamino group such as methoxycarbonylamino, trifluoromethoxycarbonylamino and the like, etc.), (25) an oxo group, (26) a 5- to 7-membered cyclic amino optionally having substituent(s), (27) a heterocyclic group, (28) a C₁₋₃ alkylenedioxy (e.g., methylenedioxy, ethylenedioxy, etc.), (29) a carbamoyl group, (30) a group which is a combination of 1 to 3 groups from the above-mentioned (1)-(29) and the like can be used.

The “hydrocarbon group” of the “hydrocarbon group optionally having substituent(s)” may have 1 to 5, preferably 1 to 3, of the above-mentioned substituents at substitutable position(s) for the hydrocarbon group. When the number of the substituents is two or more, each substituent may be the same or different.

The “acyl” as the “substituent” of the “hydrocarbon group optionally having substituent(s)” represented by R¹, for example, includes formyl, C₁₋₆ alkyl-carbonyl (e.g., acetyl, propionyl, etc.), heterocyclyl-C₁₋₆ alkyl-carbonyl, C₃₋₇ cycloalkyl-carbonyl (e.g., cyclopropylcarbonyl, etc.), C₆₋₁₄ aryl-carbonyl (e.g., phenylcarbonyl, naphthylcarbonyl, etc.), heterocyclylcarbonyl (e.g., nicotinoyl, etc.), C₁₋₆ alkoxy-carbonyl (e.g., methoxycarbonyl, ethoxycarbonyl, propoxycarbonyl, butoxycarbonyl, etc.), C₆₋₁₄ aryloxy-carbonyl (e.g., phenoxycarbonyl, naphthoxycarbonyl, etc.), heterocyclyloxy-carbonyl, C₁₋₆ alkylsulfonyl (e.g., methylsulfonyl, ethylsulfonyl, etc.), C₆₋₁₄ arylsulfonyl (e.g., phenylsulfonyl, 2-naphthylsulfonyl, 1-naphthylsulfonyl, etc.), heterocyclylsulfonyl, C₁₋₆ alkylsulfinyl (e.g., methylsulfinyl, ethylsulfinyl, propylsulfinyl, butylsulfinyl, etc.), C₆₋₁₄ arylsulfinyl (e.g., phenylsulfinyl, naphthylsulfinyl, etc.), carbamoyl, thiocarbamoyl, mono-C₁₋₆ alkyl-carbamoyl (e.g., methylcarbamoyl, ethylcarbamoyl, etc.), di-C₁₋₆ alkyl-carbamoyl (e.g., dimethylcarbamoyl, diethylcarbamoyl, etc.), C₆₋₁₄ aryl-carbamoyl (e.g., phenylcarbamoyl, naphthylcarbamoyl, etc.) and the like. Here, as the heterocyclic group of the heterocyclyl-C₁₋₆ alkyl-carbonyl, heterocyclylcarbonyl, heterocyclyloxy-carbonyl and heterocyclylsulfonyl, for example, a 5- to 14-membered (preferably 5- to 9-membered, more preferably 5- or 6-membered) non-aromatic heterocyclic group (e.g., pyrrolidinyl, tetrahydrofuryl, tetrahydrothienyl, piperidyl, tetrahydropyranyl, morpholinyl, thiomorpholinyl, piperazinyl) or aromatic heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl) containing, besides carbon atom, 1 to 4 heteroatoms of one or two kinds selected from the group consisting of a nitrogen atom, an oxygen atom and a sulfur atom, and optionally having substituent(s) such as halogen atom, optionally halogenated C₁₋₆ alkyl, C₁₋₆ alkoxy, oxo and the like, and the like can be used.

The “acyloxy” and “acylamino” recited as the “substituent” of the “hydrocarbon group optionally having substituent(s)” represented by R¹ include, for example, the same groups as those referred to herein above for the foregoing “acyloxy” and “acylamino” recited as the “substituent” of the “phenyl group” represented by Ar.

The “5- to 7-membered cyclic amino optionally having substituent(s)” recited as the “substituent” of the “hydrocarbon group optionally having substituent(s)” represented by R¹ includes, for example, the same group as those referred to herein above for the foregoing “5- to 7-membered cyclic amino optionally having substituent(s)” recited as the “substituent” of the “phenyl group” represented by Ar.

As the “heterocyclic group” recited as the “substituent” of the “hydrocarbon group optionally having substituent(s)” represented by R¹, for example, a 5- to 14-membered (preferably 5- to 9-membered, more preferably 5- or 6-membered) aromatic heterocyclic group (e.g., furyl, thienyl, pyrrolyl, oxazolyl, isoxazolyl, thiazolyl, isothiazolyl, imidazolyl, pyrazolyl, 1,2,3-oxadiazolyl, 1,2,4-oxadiazolyl, 1,3,4-oxadiazolyl, furazanyl, 1,2,3-thiadiazolyl, 1,2,4-thiadiazolyl, 1,3,4-thiadiazolyl, 1,2,3-triazolyl, 1,2,4-triazolyl, tetrazolyl, pyridyl, pyridazinyl, pyrimidinyl, pyrazinyl, triazinyl) or non-aromatic heterocyclic group (e.g., pyrrolidinyl, tetrahydrofuryl, tetrahydrothienyl, piperidyl, tetrahydropyranyl, morpholinyl, thiomorpholinyl, piperazinyl) containing, besides carbon atom, 1 to 4 heteroatoms of one or two kinds selected from the group consisting of a nitrogen atom, an oxygen atom and a sulfur atom, and the like can be mentioned. These non-aromatic heterocyclic groups may be further fused with other aromatic or non-aromatic homocyclic ring or heterocyclic ring. The “heterocyclic group” may have substituent(s) such as halogen atom, optionally halogenated C₁₋₆ alkyl, C₁₋₆ alkoxy, oxo and the like.

The “acyl group” represented by R¹ includes, for example, the same group as those referred to herein above for the foregoing “acyl” recited as the “substituent” of the “phenyl group” represented by Ar.

As the “heterocyclic group” of the “heterocyclic group optionally having substituent(s)” represented by R¹, for example, a 5- to 14-membered (preferably 5- to 10-membered) (monocyclic to tricyclic, preferably monocyclic or bicyclic) heterocyclic group containing, besides carbon atom, 1 to 4 (preferably 1 to 3) heteroatoms of one or two kinds selected from the group consisting of a nitrogen atom, an oxygen atom and a sulfur atom, and the like can be mentioned. For example, 5-membered ring groups containing, besides carbon atom, 1 to 4 heteroatoms selected from the group consisting of a nitrogen atom, an oxygen atom and a sulfur atom, such as 2- or 3-thienyl, 2- or 3-furyl, 1-, 2- or 3-pyrrolyl, 1-, 2- or 3-pyrrolidinyl, 2-, 4- or 5-oxazolyl, 3-, 4- or 5-isoxazolyl, 2-, 4- or 5-thiazolyl, 3-, 4- or 5-isothiazolyl, 3-, 4- or 5-pyrazolyl, 2-, 3- or 4-pyrazolidinyl, 2-, 4- or 5-imidazolyl, 1,2,3-triazolyl, 1,2,4-triazolyl, 1H- or 2H-tetrazolyl and the like; 6-membered ring groups containing, besides carbon atom, 1 to 4 heteroatoms selected from the group consisting of a nitrogen atom, an oxygen atom and a sulfur atom, such as 2-, 3- or 4-pyridyl, N-oxide-2-, 3- or 4-pyridyl, 2-, 4- or 5-pyrimidinyl, N-oxide-2-, 4- or 5-pyrimidinyl, thiomorpholinyl, morpholinyl, piperidino, 2-, 3- or 4-piperidyl, thiopyranyl, 1,4-oxazinyl, 1,4-thiazinyl, 1,3-thiazinyl, piperazinyl, triazinyl, 3- or 4-pyridazinyl, pyrazinyl, N-oxide-3- or 4-pyridazinyl and the like; bicyclic or tricyclic fused ring groups containing, besides carbon atom, 1 to 4 heteroatoms selected from the group consisting of a nitrogen atom, an oxygen atom and a sulfur atom, such as indolyl, benzofuryl, benzothiazolyl, benzoxazolyl, benzimidazolyl, quinolyl, isoquinolyl, phthalazinyl, quinazolinyl, quinoxalinyl, indolizinyl, quinolizinyl, 1,8-naphthyridinyl, dibenzofuranyl, carbazolyl, acrydinyl, phenanthridinyl, chromanyl, phenothiazinyl, phenoxazinyl and the like (preferably, a group formed by condensation of the above-mentioned 5- or 6-membered ring with one or two 5- or 6-membered rings containing, besides carbon atom, 1 to 4 heteroatoms selected from the group consisting of a nitrogen atom, an oxygen atom and a sulfur atom) and the like can be used. Of these, a 5- to 7-membered (preferably 5- or 6-membered) heterocyclic group containing, besides carbon atom, 1 to 3 heteroatoms selected from the group consisting of a nitrogen atom, an oxygen atom and a sulfur atom is preferable.

As the substituent that the “heterocyclic group” of the “heterocyclic group optionally having substituent(s)” may have, those similar to the “substituent” that the “hydrocarbon group” of the above-mentioned “hydrocarbon group optionally having substituent(s)” may have can be used and, for example, (1) a halogen atom (e.g., fluorine, chlorine, bromine, iodine, etc.), (2) a lower alkyl group (e.g., a C₁₋₆ alkyl group such as methyl, ethyl, propyl, isopropyl, butyl, isobutyl, sec-butyl, tert-butyl, pentyl, hexyl and the like, etc.), (3) a cycloalkyl group (e.g., a C₃₋₆ cycloalkyl group such as cyclopropyl, cyclobutyl, cyclopentyl, cyclohexyl and the like, etc.), (4) a lower alkynyl group (e.g., a C₂₋₆ alkynyl group such as ethynyl, 1-propynyl, propargyl and the like, etc.), (5) a lower alkenyl group (e.g., a C₂₋₆ alkenyl group such as vinyl, allyl, isopropenyl, butenyl, isobutenyl and the like, etc.), (6) an aralkyl group (e.g., a C₇₋₁₁ aralkyl group such as benzyl, α-methylbenzyl, phenethyl and the like, etc.), (7) an aryl group (e.g., a C₆₋₁₀ aryl group such as phenyl, naphthyl and the like, etc., preferably phenyl group, etc.), (8) a lower alkoxy group (e.g., a C₁₋₆ alkoxy group such as methoxy, ethoxy, propoxy, isopropoxy, butoxy, isobutoxy, sec-butoxy, tert-butoxy and the like, etc.), (9) an aryloxy group (e.g., a C₆₋₁₀ aryloxy group such as phenoxy and the like, etc.), (10) an acyl (e.g., formyl group, lower alkyl-carbonyl group (e.g., C₁₋₆ alkyl-carbonyl group such as acetyl, propionyl, butyryl, isobutyryl and the like, etc.), arylcarbonyl group (e.g., C₆₋₁₄ aryl-carbonyl group such as benzoyl, naphthoyl and the like, etc.), carbamoyl group, sulfo group, sulfino group, phosphono group, sulfamoyl group, lower alkylsulfinyl group (e.g., C₁₋₆ alkylsulfinyl group such as methylsulfinyl, ethylsulfinyl, propylsulfinyl, butylsulfinyl and the like, etc.), arylsulfinyl group (e.g., C₆₋₁₄ arylsulfinyl group such as phenylsulfinyl, naphthylsulfinyl and the like, etc.), lower alkylsulfonyl group (e.g., C₁₋₆ alkylsulfonyl group such as methylsulfonyl, ethylsulfonyl, propylsulfonyl, butylsulfonyl and the like, etc.), arylsulfonyl group (e.g., C₆₋₁₄ arylsulfonyl group such as phenylsulfonyl, naphthylsulfonyl and the like, etc.), monoalkylsulfamoyl group (e.g., mono-C₁₋₆ alkylsulfamoyl group such as N-methylsulfamoyl, N-ethylsulfamoyl, N-propylsulfamoyl, N-isopropylsulfamoyl, N-butylsulfamoyl and the like, etc.), dialkylsulfamoyl group (e.g., di-C₁₋₆ alkylsulfamoyl group such as N,N-dimethylsulfamoyl, N,N-diethylsulfamoyl, N,N-dipropylsulfamoyl, N,N-dibutylsulfamoyl and the like, etc.) and the like), (11) a carboxyl group, (12) an acyloxy (e.g., formyloxy, lower alkyl-carbonyloxy group (e.g., C₁₋₆ alkyl-carbonyloxy group such as acetyloxy, propionyloxy, butyryloxy, isobutyryloxy and the like, etc.), arylcarbonyloxy group (e.g., C₆₋₁₄ aryl-carbonyloxy group such as benzoyloxy, naphthoyloxy and the like, etc.), lower alkoxycarbonyl group (e.g., C₁₋₆ alkoxy-carbonyl group such as methoxycarbonyl, ethoxycarbonyl, propoxycarbonyl, isopropoxycarbonyl, butoxycarbonyl, isobutoxycarbonyl, tert-butoxycarbonyl and the like, etc.), aralkyloxycarbonyl (e.g., C₇₋₁₅ aralkyloxycarbonyl group such as benzyloxycarbonyl and the like, etc.), (13) a mono-, di- or trihalogeno-lower alkyl group (e.g., mono-, di- or trihalogeno-C₁₋₆ alkyl group such as chloromethyl, dichloromethyl, trifluoromethyl, 2,2,2-trifluoroethyl and the like, etc.), (14) an oxo group, (15) an amidino group, (16) an imino group, (17) an amino group, (18) a mono-lower alkylamino group (e.g., mono-C₁₋₆ alkylamino group such as methylamino, ethylamino, propylamino, isopropylamino, butylamino and the like, etc.), (19) a di-lower alkylamino group (e.g., di-C₁₋₄ alkylamino group such as dimethylamino, diethylamino, dipropylamino, diisopropylamino, dibutylamino, methylethylamino and the like, etc.), (20) an acylamino, (21) a 3- to 6-membered cyclic amino group optionally containing, besides carbon atom and one nitrogen atom, 1 to 3 heteroatoms selected from the group consisting of an oxygen atom, a sulfur atom and a nitrogen atom (e.g., 3- to 6-membered cyclic amino group such as aziridinyl, azetidinyl, pyrrolidinyl, pyrrolinyl, pyrrolyl, imidazolyl, pyrazolyl, imidazolidinyl, piperidyl, morpholinyl, dihydropyridyl, tetrahydropyridyl, N-methylpiperazinyl, N-ethylpiperazinyl and the like, etc.), (22) an alkylenedioxy group (e.g., C₁₋₃ alkylenedioxy group such as methylenedioxy, ethylenedioxy and the like, etc.), (23) a hydroxy group, (24) a nitro group, (25) a cyano group, (26) a mercapto group, (27) an alkylthio group (e.g., C₁₋₆ alkylthio group such as methylthio, ethylthio, propylthio, isopropylthio, butylthio, sec-butylthio, tert-butylthio and the like, etc.), (28) an arylthio group (e.g., C₆₋₁₄ arylthio group such as phenylthio, naphthylthio and the like, etc.), (29) a group which is a combination of 1 to 3 groups from the above-mentioned (1)-(28) and the like can be used. As used herein, as the “acyl”, “acyloxy” and “acylamino”, those similar to the “acyl”, “acyloxy” and “acylamino” recited as the “substituent” that the “hydrocarbon group” of the above-mentioned “hydrocarbon group optionally having substituent(s)” may have can be used.

The “heterocyclic group” of the “heterocyclic group optionally having substituent(s)” may have 1 to 5, preferably 1 to 3, the above-mentioned substituents at substitutable position(s) for the heterocyclic group. When the number of the substituents-is two or more, each substituent may be the same or different.

R¹ is preferably a hydrogen atom or an acyl group. As the acyl group, a group represented by —(C═O)—R⁵, —SO₂—R⁵ or -(C═O)—(C═O)—R⁵ (R⁵ is a hydrogen atom, a hydrocarbon group optionally having substituent(s), an amino group optionally having substituent(s), a hydroxy group optionally having a substituent or a heterocyclic group optionally having substituent(s)) is preferable.

The “hydrocarbon group optionally having substituent(s)”, “amino group optionally having substituent(s)”, “hydroxy group optionally having a substituent” and “heterocyclic group optionally having substituent(s)” represented by R⁵, include, for example, the same groups as those referred to herein above for the foregoing “hydrocarbon group optionally having substituent(s)”, “amino group optionally having substituent(s)”, “hydroxy group optionally having a substituent” and “heterocyclic group optionally having substituent(s)” represented by R³.

As the “hydrocarbon group optionally having substituent(s)” represented by R⁵;
(1) a C₁₋₆ alkyl group (e.g., methyl group) optionally having 1 or 2 substituents selected from the group consisting of (i) amino, (ii) C₁₋₆ alkoxy (e.g., methoxy), (iii) formylamino, (iv) C₁₋₆ alkyl-carbonylamino (e.g., acetylamino), (v) C₁₋₆ alkoxy-carbonylamino (e.g., methoxycarbonylamino, tert-butoxycarbonylamino), (vi) C₁₋₆ alkylsulfonylamino (e.g., methylsulfonylamino), (vii) a heterocyclic group (e.g., a 5- or 6-membered aromatic or non-aromatic heterocyclic group (e.g., piperazinyl, oxopiperazinyl, 1-piperidinyl, imidazolyl, tetrazolyl, triazolyl, dihydrotriazolyl, oxazolidinyl, imidazolidinyl, tetrahydropyrimidinyl, pyrrolidinyl and the like) containing, besides carbon atom, 1 to 4 heteroatoms of one or two kinds selected from the group consisting of a nitrogen atom, an oxygen atom and a sulfur atom) optionally having 1 to 5 substituents selected from the group consisting of C₁₋₆ alkyl (e.g., methyl, isopropyl), hydroxy, formyl, C₁₋₆ alkyl-carbonyl (e.g., acetyl), formylamino and C₁₋₆ alkyl-carbonylamino (e.g., acetylamino), optionally having 1 or 2 oxo, and said heterocyclic group optionally forms a spiro ring together with cyclopentane or cyclohexane, (viii) C₁₋₆ alkyl-carbonyloxy (e.g., acetoxy), (ix) hydroxy and (x) carbamoyl and the like,
(2) a C₃₋₇ cycloalkyl group (e.g., cyclohexyl group) optionally having substituent(s) selected from the group consisting of (i) amino, (ii) C₁₋₆ alkoxy (e.g., methoxy), (iii) formylamino, (iv) C₁₋₆ alkyl-carbonylamino (e.g., acetylamino), (v) C₁₋₆ alkoxy-carbonylamino (e.g., methoxycarbonylamino, tert-butoxycarbonylamino), (vi) C₁₋₆ alkylsulfonylamino (e.g., methylsulfonylamino), (vii) a heterocyclic group (e.g., 5- or 6-membered aromatic or non-aromatic heterocyclic group (e.g., piperazinyl, oxopiperazinyl, 1-piperidinyl, imidazolyl, tetrazolyl, triazolyl, dihydrotriazolyl, oxazolidinyl, imidazolidinyl, tetrahydropyrimidinyl, pyrrolidinyl and the like) containing, besides carbon atom, 1 to 4 heteroatoms of one or two kinds selected from the group consisting of a nitrogen atom, an oxygen atom and a sulfur atom) optionally having 1 to 5 substituents selected from the group consisting of C₁₋₆ alkyl (e.g., methyl, isopropyl), hydroxy, formyl, C₁₋₆ alkyl-carbonyl (e.g., acetyl), formylamino and C₁₋₆ alkyl-carbonylamino (e.g., acetylamino), and optionally having 1 or 2 oxo, (viii) C₁₋₆ alkyl-carbonyloxy (e.g., acetoxy), (ix) hydroxy and (x) carbamoyl and the like,
(3) a C₆₋₁₄ aryl group (e.g., phenyl group) optionally having substituent(s) selected from the group consisting of (i) formylamino and (ii) C₁₋₆ alkyl-carbonylamino (e.g., acetylamino) and the like, and the like are preferable.

As the “amino group optionally having substituent(s)” represented by R⁵, an amino group, a C₁₋₆ alkylamino group, a di-C₁₋₆ alkylamino group and the like are preferable.

As the “hydroxy group optionally having a substituent” represented by R⁵, a C₁₋₆ alkoxy group is preferable.

As the “heterocyclic group optionally having substituent(s)” represented by R⁵,
a 5- or 6-membered non-aromatic heterocyclic group (particularly, 1-piperidyl group, 4-piperidyl group, piperazinyl group) containing, besides carbon atom, 1 or 2 nitrogen atoms, and optionally having 1 or 2 substituents selected from
(i) a C₁₋₆ alkyl group (e.g., methyl, ethyl, propyl, isopropyl) optionally substituted by substituent(s) selected from the group consisting of formyl, C₁₋₆ alkyl-carbonyl (e.g., acetyl), C₁₋₆ alkoxy-carbonyl (e.g., methoxycarbonyl, isopropoxycarbonyl), carbamoyl and mono or di-C₁₋₆ alkyl-carbamoyl (e.g., dimethylcarbamoyl),
(ii) a formyl group,
(iii) a C₁₋₆ alkyl-carbonyl group (e.g., acetyl) optionally substituted by substituent(s) selected from the group consisting of (a) formylamino, (b) C₁₋₆ alkyl-carbonylamino (e.g., acetylamino) and (c) a 5- or 6-membered non-aromatic or aromatic heterocyclic group (e.g., tetrazolyl, triazolyl, dihydrotriazolyl, etc.) containing, besides carbon atom, 1 to 4 heteroatoms of one or two kinds selected from the group consisting of a nitrogen atom, an oxygen atom and a sulfur atom, and optionally having 1 or 2 oxo,
(iv) a-C₁₋₆ alkylsulfonyl group (e.g., methylsulfonyl) optionally substituted by substituent(s) selected from the group consisting of (a) formylamino, (b) C₁₋₆ alkyl-carbonylamino (e.g., acetylamino) and (c) a 5- or 6-membered non-aromatic or aromatic heterocyclic group (e.g., tetrazolyl, triazolyl, dihydrotriazolyl, etc.) containing, besides carbon atom, 1 to 4 heteroatoms of one or two kinds selected from the group consisting of a nitrogen atom, an oxygen atom and a sulfur atom, and optionally having 1 or 2 oxo,
(v) a C₁₋₆ alkoxy-carbonyl group (e.g., methoxycarbonyl, tert-butoxycarbonyl, etc.) optionally substituted by substituent(s) selected from the group consisting of (a) formylamino, (b) C₁₋₆ alkyl-carbonylamino (e.g., acetylamino) and (c) a 5- or 6-membered non-aromatic or aromatic heterocyclic group (e.g., tetrazolyl; triazolyl, dihydrotriazolyl, etc.) containing, besides carbon atom, 1 to 4 heteroatoms of one or two kinds selected from the group consisting of a nitrogen atom, an oxygen atom and a sulfur atom, and optionally having 1 or 2 oxo,
(vi) a substituent bonded via carbon atom, such as a heterocyclyl-carbonyl group (e.g., a 5- or 6-membered aromatic or non-aromatic heterocyclyl (e.g., imidazoline)-carbonyl group containing, besides carbon atom, 1 to 4 heteroatoms of one or two kinds selected from the group consisting of a nitrogen atom, an oxygen atom and a sulfur atom) optionally having 1 or 2 oxo, and the like,
(vii) a heterocyclic group optionally substituted by oxo (e.g., a 5- or 6-membered aromatic or non-aromatic heterocyclic group (e.g., dihydrofuran-2(3H)-one) containing, besides carbon atom, 1 to 4 heteroatoms of one or two kinds selected from the group consisting of a nitrogen atom, an oxygen atom and a sulfur atom),
(viii) a di-C₁₋₆ alkyl-carbamoyl group (e.g., dimethylcarbamoyl),
(ix) oxo and the like is preferable.

Of these, as R¹,
(1) a hydrogen atom,
(2) a C₁₋₆ alkyl-carbonyl optionally having 1 or 2 substituents selected from the group consisting of (i) an amino, (ii) a C₁₋₆ alkoxy, (iii) a C₁₋₆ alkyl-carbonylamino, (iv) a C₁₋₆ alkoxy-carbonylamino, (v) a C₁₋₆ alkylsulfonylamino, (vi) a 5- or 6-membered nitrogen-containing heterocyclic group (e.g., tetrazolyl, triazolyl, dihydrotriazolyl, oxazolidinyl, imidazolidinyl, tetrahydropyrimidinyl, piperidinyl, pyrrolidinyl, etc.) optionally having 1 to 5 substituents selected from the group consisting of a C₁₋₆ alkyl and an oxo, said heterocyclic group optionally forms a spiro ring together with cyclopentane or cyclohexane, (vii) a C₁₋₆ alkyl-carbonyloxy, (viii) a hydroxy and (ix) a carbamoyl,
(3) a C₁₋₆ alkoxy-carbonyl,
(4) a C₁₋₆ alkylamino-carbonyl,
(5) a C₁₋₆ alkylsulfonyl,
(6) an aminocarbonylcarbonyl,
(7) a C₁₋₆ alkylamino-carbonylcarbonyl,
(8) a di-C₁₋₆ alkylamino-carbonylcarbonyl, or
(9) a piperidin-4-ylcarbonyl optionally having 1 or 2 substituents selected from the group consisting of (i) a C₁₋₆ alkyl-carbonyl optionally having a 5- or 6-membered nitrogen-containing heterocyclic group (e.g., tetrazolyl, triazolyl, dihydrotriazolyl, etc.) optionally having 1 or 2 oxo, (ii) a C₁₋₆ alkoxy-carbonyl, (iii) a C₁₋₆ alkylsulfonyl, (iv) a C₁₋₆ alkyl-carbonylamino-C₁₋₆ alkyl-carbonyl, (v) a di-C₁₋₆ alkyl-carbamoyl and (vi) an oxo, and the like are preferable.

As the “5- or 6-membered nitrogen-containing heterocyclic group” of the above-mentioned “5- or 6-membered nitrogen-containing heterocyclic group optionally having 1 to 5 substituents selected from the group consisting of a C₁₋₆ alkyl and an oxo, said heterocyclic group optionally forms a spiro ring together with cyclopentane or cyclohexane”, a 5- or 6-membered heterocyclic group (e.g., tetrazolyl, triazolyl, dihydrotriazolyl, oxazolidinyl, imidazolidinyl, tetrahydropyrimidinyl, piperidinyl, pyrrolidinyl, etc.) optionally containing, besides one nitrogen atom and carbon atom, 1 to 3 heteroatoms of one or two kinds selected from the group consisting of a nitrogen atom, an oxygen atom and a sulfur atom can be mentioned.

As the “5- or 6-membered nitrogen-containing heterocyclic group” of the above-mentioned “5- or 6-membered nitrogen-containing heterocyclic group optionally having 1 or 2 oxo”, a 5- or 6-membered heterocyclic group (e.g., tetrazolyl, triazolyl, dihydrotriazolyl, etc.) optionally containing, besides one nitrogen atom and carbon atom, 1 to 3 heteroatoms of one or two kinds selected from the group consisting of a nitrogen atom, an oxygen atom and a sulfur atom can be mentioned.

Of these, as R¹, C₁₋₆ alkoxy-C₁₋₆ alkyl-carbonyl, C₁₋₆ alkyl-carbonylamino-C₁₋₆ alkyl-carbonyl, C₁₋₆ alkoxy-carbonyl or 1-(C₁₋₆ alkyl-carbonyl)piperidin-4-ylcarbonyl is preferable.

R² is a hydrogen atom, a C₁₋₆ alkyl group optionally having substituent(s) or a C₃₋₆ cycloalkyl group optionally having substituent(s).

As the “C₁₋₆ alkyl group” of the “C₁₋₆ alkyl group optionally having substituent(s)” represented by R², for example, methyl, ethyl, propyl, isopropyl, butyl, isobutyl, sec-butyl, tert-butyl, pentyl, hexyl and the like can be used. Of these, a C₁₋₃ alkyl group such as methyl, ethyl and the like is preferable and a methyl group is particularly preferable.

As the “C₃₋₆ cycloalkyl group” of the “C₃₋₆ cycloalkyl group optionally having substituent(s)” represented by R², for example, cyclopropyl, cyclobutyl, cyclopentyl, cyclohexyl and the like can be used.

The substituent of the “C₁₋₆ alkyl group optionally having substituent(s)” or “C₃₋₆ cycloalkyl group optionally having substituent(s)” represented by R² include, for example, the same group as those referred to herein above for the foregoing substituent that the “hydrocarbon group” of the “hydrocarbon group optionally having substituent(s)” represented by R¹ may have. Of these, one having 1 to 3 halogen atoms (e.g., fluorine, chlorine, bromine, iodine, etc.) is preferable.

As R², a hydrogen atom or a C₁₋₆ alkyl group optionally having substituent(s) is preferable. Of these, a C₁₋₆ alkyl group optionally having 1 to 3 halogen atoms (e.g., fluorine, chlorine, bromine, iodine, etc.) is preferable. A C₁₋₆ alkyl group is particularly preferable and methyl is specifically preferable.

Z is-a methylene group optionally having a C₁₋₆ alkyl group (e.g., methyl, ethyl, propyl, isopropyl, butyl, isobutyl, sec-butyl, tert-butyl, pentyl, hexyl, etc.).

Z is preferably a methylene group optionally having a methyl group.

Ring A is a piperidine ring optionally further having substituent(s). That is, ring A may further have 1 to 8 substituents besides R¹, NH— and Ar.

As the “substituent” of the “piperidine ring optionally having substituent(s)”, those similar to the substituent of the “phenyl group” for the above-mentioned Ar can be mentioned.

Ring A is preferably a piperidine ring without a substituent other than R¹, NH— and Ar.

Ring B and ring C are benzene rings optionally further having substituent(s). That is, ring B may further have 1 to 3 substituents besides ring C, O—R² and Z-, and ring C may have 1 to 5 substituents besides ring B.

As the “substituent” that ring B and ring C may have, those similar to the substituent of the “phenyl group” for the above-mentioned Ar can be mentioned.

As ring B, a benzene ring optionally further having a halogen atom or a C₁₋₆ alkyl or ring B forming a 2,3-dihydrobenzofuran ring together with R² are preferable.

As ring C, a benzene ring optionally having 1 or 2 substituents selected from the group consisting of (1) a cyano, (2) a nitro, (3) a halogen atom, (4) a C₁₋₆ alkyl optionally having 1 to 3 halogen atoms, (5) a C₁₋₆ alkynyl, (6) a C₁₋₆ alkoxy optionally having 1 to 3 halogen atoms, (7) a C₁₋₆ alkylthio, (8) a C₁₋₆ alkylsulfonyl, (9) a di-C₁₋₆ alkylamino, (10) a C₁₋₆ alkyl-carbonyl, (11) a C₁₋₆ alkyl-carbonylamino, (12) a C₁₋₆ alkoxy-carbonyl and (13) a carbamoyl is preferable. Particularly, a benzene ring optionally having 1 or 2 substituents selected from the group consisting of (1) a cyano, (2) a halogen atom, (3) a C₁₋₆ alkyl optionally having 1 to 3 halogen atoms and (4) a C₁₋₆ alkoxy is preferable.

As compound (I), a compound represented by the formula (II) 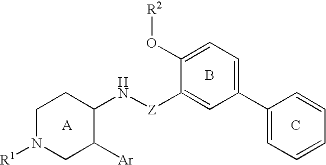
wherein the symbols are as defined above, is preferable, and compound (IIa) having a configuration represented by the formula 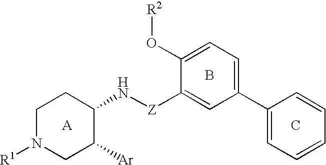
wherein the symbols are as defined above is particularly preferable. More specifically, compound (IIa) wherein Ar is a phenyl group optionally having 1 to 3 halogen atoms;
R¹ is (1) a hydrogen atom,
(2) a C₁₋₆ alkyl-carbonyl optionally having 1 or 2 substituents selected from the group consisting of (i) an amino, (ii) a C₁₋₆ alkoxy, (iii) a C₁₋₆ alkyl-carbonylamino, (iv) a C₁₋₆ alkoxy-carbonylamino, (v) a C₁₋₆ alkylsulfonylamino, (vi) a 5- or 6-membered nitrogen-containing heterocyclic group optionally having 1 to 5 substituents selected from the group consisting of a C₁₋₆ alkyl and an oxo, said heterocyclic group optionally forms a spiro ring together with cyclopentane or cyclohexane, (vii) a C₁₋₆ alkyl-carbonyloxy, (viii) a hydroxy and (ix) a carbamoyl,
(3) a C₁₋₆ alkoxy-carbonyl,
(4) a C₁₋₆ alkylamino-carbonyl,
(5) a C₁₋₆ alkylsulfonyl,
(6) an aminocarbonylcarbonyl,
(7) a C₁₋₆ alkylamino-carbonylcarbonyl,
(8) a di-C₁₋₆ alkylamino-carbonylcarbonyl, or
(9) a piperidin-4-ylcarbonyl optionally having 1 or 2 substituents selected from the group consisting of (i) a C₁₋₆ alkyl-carbonyl optionally having a 5- or 6-membered nitrogen-containing heterocyclic group optionally having 1 or 2 oxo, (ii) a C₁₋₆ alkoxy-carbonyl, (iii) a C₁₋₆ alkylsulfonyl, (iv) a C₁₋₆ alkyl-carbonylamino-C₁₋₆ alkyl-carbonyl, (v) a di-C₁₋₆ alkyl-carbamoyl and (vi) an oxo;
R² is (1) a hydrogen atom or (2) a C₁₋₆ alkyl group optionally having 1 to 3 halogen atoms;
Z is a methylene group optionally having a methyl group;
ring A is a piperidine ring without a further substituent;
ring B is a benzene ring optionally further having a halogen atom or a C₁₋₆ alkyl or ring B forms a 2,3-dihydrobenzofuran ring together with R²; and
ring C is a benzene ring optionally having 1 or 2 substituents selected from the group consisting of
(1) a cyano,
(2) a nitro,
(3) a halogen atom,
(4) a C₁₋₆ alkyl optionally having 1 to 3 halogen atoms,
(5) a C₁₋₆ alkynyl,
(6) a C₁₋₆ alkoxy optionally having 1 to 3 halogen atoms,
(7) a C₁₋₆ alkylthio,
(8) a C₁₋₆ alkylsulfonyl,
(9) a di-C₁₋₆ alkylamino,
(10) a C₁₋₆ alkyl-carbonyl,
(11) a C₁₋₆ alkyl-carbonylamino,
(12) a C₁₋₆ alkoxy-carbonyl and
(13) a carbamoyl
   is preferable. More preferably, compound (IIa) wherein
Ar is a phenyl group;
R¹ is C₁₋₆ alkoxy-C₁₋₆ alkyl-carbonyl, C₁₋₆ alkyl-carbonylamino-C₁₋₆ alkyl-carbonyl, C₁₋₆ alkoxy-carbonyl or 1-(C₁₋₆ alkyl-carbonyl)piperidin-4-ylcarbonyl;
ring A is a piperidine ring without a further substituent;
R² is a C₁₋₆ alkyl group optionally having 1 to 3 halogen atoms;
ring B is a benzene ring without a further substituent; and
ring C is a benzene ring optionally having 1 or 2 substituents selected from the group consisting of (1) a cyano, (2) a halogen atom, (3) a C₁₋₆ alkyl optionally having 1 to 3 halogen atoms and (4) a C₁₋₆ alkoxy can be mentioned.

Moreover, as compound (I), a compound represented by the formula (IIa′) 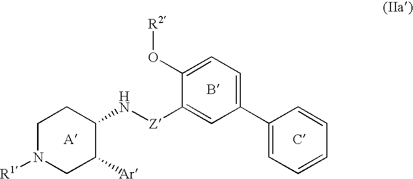
wherein
Ar′ is a phenyl group optionally having 1 to 3 halogen atoms;
R¹′ is (1) a hydrogen atom,
(2) a C₁₋₆ alkyl-carbonyl optionally having 1 or 2 substituents selected from the group consisting of (i) an amino, (ii) a C₁₋₆ alkoxy, (iii) a C₁₋₆ alkyl-carbonylamino, (iv) a C₁₋₆ alkoxy-carbonylamino, (v) a C₁₋₆ alkylsulfonylamino, (vi) a 5- or 6-membered nitrogen-containing heterocyclic group optionally having 1 to 5 substituents selected from the group consisting of a C₁₋₆ alkyl and an oxo, said heterocyclic group optionally forms a spiro ring together with cyclopentane or cyclohexane, (vii) a C₁₋₆ alkyl-carbonyloxy, (viii) a hydroxy and (ix) a carbamoyl,
(3) a C₁₋₆ alkoxy-carbonyl,
(4) a C₁₋₆ alkylsulfonyl,
(5) an aminocarbonylcarbonyl,
(6) a C₁₋₆ alkylamino-carbonylcarbonyl,
(7) a di-C₁₋₆ alkylamino-carbonylcarbonyl, or
(8) a piperidin-4-ylcarbonyl optionally having 1 or 2 substituents selected from the group consisting of (i) a C₁₋₆ alkyl-carbonyl optionally having a 5- or 6-membered nitrogen-containing heterocyclic group optionally having 1 or 2 oxo, (ii) a C₁₋₆ alkoxy-carbonyl, (iii) a C₁₋₆ alkylsulfonyl, (iv) a C₁₋₆ alkyl-carbonylamino-C₁₋₆ alkyl-carbonyl, (v) a di-C₁₋₆ alkyl-carbamoyl and (vi) an oxo;
R²′ is (1) a hydrogen atom or (2) a C₁₋₆ alkyl group optionally having 1 to 3 halogen atoms;
Z′ is a methylene group optionally having a methyl group;
ring A′ is a piperidine ring without a further substituent;
ring B′ is a benzene ring optionally further having a halogen atom or a C₁₋₆ alkyl or ring B′ forms a 2,3-dihydrobenzofuran ring together with R²′;
ring C′ is a benzene ring optionally having 1 or 2 substituents selected from the group consisting of
(1) a cyano,
(2) a nitro,
(3) a halogen atom,
(4) a C₁₋₆ alkyl optionally having 1 to 3 halogen atoms,
(5) a C₁₋₆ alkynyl,
(6) a C₁₋₆ alkoxy optionally having 1 to 3 halogen atoms,
(7) a C₁₋₆ alkylthio,
(8) a C₁₋₆ alkylsulfonyl,
(9) a di-C₁₋₆ alkylamino,
(10) a C₁₋₆ alkyl-carbonyl,
(11) a C₁₋₆ alkyl-carbonylamino,
(12) a C₁₋₆ alkoxy-carbonyl and
(13) a carbamoyl
   an also be mentioned as a preferable embodiment.

Furthermore, as compound (I), the compounds of the below-mentioned Examples 1-137, salts thereof and the like can be mentioned as preferable embodiments. Particularly, N-[2-((3R,4S)-4-{[(4′-cyano-4-methoxybiphenyl-3-yl)methyl]amino}-3-phenylpiperidin-1-yl)-2-oxoethyl]acetamide (Example 10),
N-[2-((3R,4S)-4-{[(4′-cyano-2′-fluoro-4-methoxybiphenyl-3-yl)methyl]amino}-3-phenylpiperidin-1-yl)-2-oxoethyl]acetamide (Example 11),
N-[2-((3R,4S)-4-{[(4′-chloro-4-methoxybiphenyl-3-yl)methyl]amino}-3-phenylpiperidin-1-yl)-2-oxoethyl]acetamide (Example 15),
N-[2-((3R,4S)-4-{[(4′-fluoro-4-methoxybiphenyl-3-yl)methyl]amino}-3-phenylpiperidin-1-yl)-2-oxoethyl]acetamide (Example 16),
N-[2-((3R,4S)-4-{[(4-methoxy-4′-methylbiphenyl-3-yl)methyl]amino}-3-phenylpiperidin-1-yl)-2-oxoethyl]acetamide (Example 17),
N-{2-[(3R,4S)-4-({[4-methoxy-4′-(trifluoromethyl)biphenyl-3-yl]methyl}amino)-3-phenylpiperidin-1-yl]-2-oxoethyl}acetamide (Example 18),
N-[2-((3R,4S)-4-{[(4′-cyano-3′-fluoro-4-methoxybiphenyl-3-yl)methyl]amino}-3-phenylpiperidin-1-yl)-2-oxoethyl]acetamide (Example 21),
N-[2-((3R,4S)-4-{[(4′-bromo-4-methoxybiphenyl-3-yl)methyl]amino}-3-phenylpiperidin-1-yl)-2-oxoethyl]acetamide (Example 22),
N-[2-((3R,4S)-4-{[(4′-ethynyl-4-methoxybiphenyl-3-yl)methyl]amino}-3-phenylpiperidin-1-yl)-2-oxoethyl]acetamide (Example 23),
N-{2-[(3R,4S)-4-({[4′-cyano-4-(trifluoromethoxy)biphenyl-3-yl]methyl}amino)-3-phenylpiperidin-1-yl]-2-oxoethyl}acetamide (Example 24),
N-[2-((3R,4S)-4-{[(4′-cyano-4-methoxy-2′-methylbiphenyl-3-yl)methyl]amino}-3-phenylpiperidin-1-yl)-2-oxoethyl]acetamide (Example 25),
4′-methoxy-3′-({[(3R,4S)-1-(methoxyacetyl)-3-phenylpiperidin-4-yl]amino}methyl)biphenyl-4-carbonitrile (Example 32),
2-fluoro-4′-methoxy-3′-({[(3R,4S)-1-(methoxyacetyl)-3-phenylpiperidin-4-yl]amino}methyl)biphenyl-4-carbonitrile (Example 33),
3′-[({(3R,4S)-1-[(1-acetylpiperidin-4-yl)carbonyl]-3-phenylpiperidin-4-yl}amino)methyl]-4′-methoxybiphenyl-4-carbonitrile (Example 35),
3′-[({(3R,4S)-1-[(1-acetylpiperidin-4-yl)carbonyl]-3-phenylpiperidin-4-yl}amino)methyl]-2-fluoro-4′-methoxybiphenyl-4-carbonitrile (Example 36),
(3R)-3-(acetylamino)-4-((3R,4S)-4-{[(4′-cyano-4-methoxybiphenyl-3-yl)methyl]amino}-3-phenylpiperidin-1-yl)-4-oxobutanamide (Example 41),
3′-[({(3R,4S)-1-[(2,6-dioxopiperidin-4-yl)carbonyl]-3-phenylpiperidin-4-yl}amino)methyl]-4′-methoxybiphenyl-4-carbonitrile (Example 42),
4′-methoxy-3′-({[(3R,4S)-3-phenyl-1-(1H-tetrazol-1-ylacetyl)piperidin-4-yl]amino}methyl)biphenyl-4-carbonitrile (Example 43),
4′-methoxy-3′-[({(3R,4S)-1-[(5-oxo-4,5-dihydro-1H-1,2,4-triazol-3-yl)acetyl]-3-phenylpiperidin-4-yl}amino)methyl]biphenyl-4-carbonitrile (Example 44),
N-{2-[(3R,4S)-4-({[4′-chloro-4-(trifluoromethoxy)biphenyl-3-yl]methyl}amino)-3-phenylpiperidin-1-yl]-2-oxoethyl}acetamide (Example 72),
3′-[({(3R,4S)-1-[(5,5-dimethyl-2,4-dioxo-1,3-oxazolidin-3-yl)acetyl]-3-phenylpiperidin-4-yl}amino)methyl]-2-fluoro-4′-(trifluoromethoxy)biphenyl-4-carbonitrile (Example 96, 97),
2-fluoro-3′-({[(3R,4S)-1-glycoloyl-3-phenylpiperidin-4-yl]amino}methyl)-4′-(trifluoromethoxy)biphenyl-4-carbonitrile (Example 100),
3′-[({(3R,4S)-1-[(1-acetylpiperidin-4-yl)carbonyl]-3-phenylpiperidin-4-yl}amino)methyl]-2-fluoro-4′-(trifluoromethoxy)biphenyl-4-carbonitrile (Example 101),
3′-[({(3R,4S)-1-[(2,6-dioxopiperidin-4-yl)carbonyl]-3-phenylpiperidin-4-yl}amino)methyl]-2-fluoro-4′-(trifluoromethoxy)biphenyl-4-carbonitrile (Example 102),
2-[(3R,4S)-4-({[4′-cyano-2′-fluoro-4-(trifluoromethoxy)biphenyl-3-yl]methyl}amino)-3-phenylpiperidin-1-yl]-2-oxoacetamide (Example 104),
3-{2-[(3R,4S)-4-({[4′-chloro-4-(trifluoromethoxy)biphenyl-3-yl]methyl}amino)-3-phenylpiperidin-1-yl]-2-oxoethyl}-5,5-dimethyl-1;3-oxazolidine-2,4-dione (Example 116),
4-{[(3R,4S)-4-({[4′-chloro-2′-fluoro-4-(trifluoromethoxy)biphenyl-3-yl]methyl}amino)-3-phenylpiperidin-1-yl]carbonyl}piperidine-2,6-dione (Example 132) and
3′-[({(3R,4S)-1-[(5,5-dimethyl-2,4-dioxo-1,3-oxazolidin-3-yl)acetyl]-3-phenylpiperidin-4-yl}amino)methyl]-4′-(trifluoromethoxy)biphenyl-4-carbonitrile (Example 137), salts thereof or the like can be preferably used.

A salt of compound (I) includes, for example, a metal salt, an ammonium salt, a salt with an organic base, a salt with an inorganic acid, a salt with an organic acid, a salt with basic or acidic amino acid, etc. Suitable examples of the metal salt include an alkali metal salt such as a sodium salt, a potassium salt, etc.; an alkaline earth metal salt such as a calcium salt, a magnesium salt, a barium salt, etc.; an aluminum salt, etc. Suitable examples of the salts with an organic base include salts with trimethylamine, triethylamine, pyridine, picoline, 2,6-lutidine, ethanolamine, diethanolamine, triethanolamine, cyclohexylamine, dicyclohexylamine, N,N′-dibenzylethylenediamine, etc. Suitable examples of the salts with an inorganic acid include salts with hydrochloric acid, hydrobromic acid, nitric acid, sulfuric acid, phosphoric acid, etc. Suitable examples of the salts with an organic acid include salts with formic acid, acetic acid, trifluoroacetic acid, phthalic acid, fumaric acid, oxalic acid, tartaric acid, maleic acid, citric acid, succinic acid, malic acid, methanesulfonic acid, benzenesulfonic acid, p-toluenesulfonic acid, etc. Suitable examples of the salts with basic amino acid include salts with arginine, lysine, ornithine, etc. Suitable examples of the salts with acidic amino acid include salts with aspartic acid and glutamic acid, etc.

Among these, pharmaceutically acceptable salts are preferred. For example, if the compound has acidic functional group, the preferred are inorganic salts such as an alkali metal salt (e.g., sodium salt, potassium salt, etc.), an alkaline earth metal salt (e.g., calcium salt, magnesium salt, barium salt, etc.), an ammonium salt, etc. If the compound has a basic functional group, the preferred are salts with an inorganic acid such as hydrochloric acid, hydrobromic acid, nitric acid, sulfuric acid, phosphoric acid, etc., or salts with an organic acid such as acetic acid, phthalic acid, fumaric acid, oxalic acid, tartaric acid, maleic acid, citric acid, succinic acid, methanesulfonic acid, p-toluenesulfonic acid, etc.

The prodrug of compound (I) of the present invention or a salt thereof means a compound which is converted to compound (I) of the present invention under the physiological condition in the living body by a reaction with an enzyme, a gastric acid, or the like, that is, by enzymatic oxidation, reduction, hydrolysis, etc.; by hydrolysis with gastric acid, etc.

The prodrug of compound (I) of the present invention includes a compound wherein the amino group of compound (I) is modified with acyl, alkyl or phosphoryl (e.g., a compound wherein the amino group of compound (I) of the present invention is modified with eicosanyl, alanyl, pentylaminocarbonyl, (5-methyl-2-oxo-1,3-dioxolen-4-yl)methoxycarbonyl, tetrahydrofuranyl, pyrrolidylmethyl, pivaloyloxymethyl or tert-butyl, etc.); a compound wherein the hydroxy group of compound (I) of the present invention is modified with acyl, alkyl, phosphoric acid or boric acid (e.g., a compound wherein the hydroxy group of compound (I) of the present invention is modified with acetyl, palmitoyl, propanoyl, pivaloyl, succinyl, fumaryl, alanyl or dimethylaminomethylcarbonyl, etc.); a compound wherein a carboxyl group of compound (I) of the present invention is modified to ester or amide (e.g., a compound wherein a carboxyl group of compound (I) of the present invention is modified to ethyl ester, phenyl ester, carboxymethyl ester, dimethylaminomethyl ester, pivaloyloxymethyl ester, ethoxycarbonyloxyethyl ester, phthalidyl ester, (5-methyl-2-oxo-1,3-dioxolen-4-yl)methyl ester, cyclohexyloxycarbonylethyl ester or methylamide, etc.); and the like. These prodrugs can be produced from compound (I) of the present invention by a method known per se.

In addition, the prodrug of compound (I) of the present invention may be a compound, which is converted into compound (I) of the present invention under the physiological conditions, as described in “Pharmaceutical Research and Development”, Vol. 7 (Drug Design), pp. 163-198 (1990), published by Hirokawa Publishing Co.

A solvate, for example, hydrate of the compound represented by the formula (I) and a salt thereof are all included in the scope of the present invention. The compound represented by the formula (I) may be labeled with an isotope (e.g., ³H, ¹⁴C, ³⁵S, ¹²⁵I, etc.) and the like.

If compound (I) according to the present invention has chiral center, isomers such as an enantiomer or a diastereomer may exist. Such isomers and a mixture thereof are all included in the scope of the present invention. In addition, there can be instances where the isomers by conformation are generated in cases, but such isomers or a mixture thereof are also included in compound (I) of the present invention or a salt thereof. Compound (I) is preferably a cis-isomer in view of the activity.

The methods for preparing compound (I) of the present invention or a salt thereof will be explained in the following.

The compound (I) of the present invention and a salt thereof can be produced according to the production method described in WO03/101964. Specifically, they can be produced using the following Method A, Method B, Method C or Method D.

[Method A]

Compound (I) can be produced by reacting a compound represented by the formula (Ib): 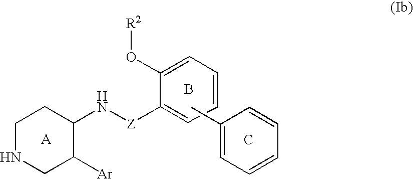
wherein each symbol is as defined above,
or a salt thereof (hereinafter to be referred to as compound (Ib)) with a compound represented by the formula (III):
R^{1a}—OH (III)
wherein R^{1a} is a hydrocarbon group optionally having substituent(s), an acyl group or a heterocyclic group optionally having substituent(s), or a salt thereof (hereinafter to be referred to as compound (III)) or a reactive derivative thereof, which is an acylating agent or alkylating agent.

As “a hydrocarbon group optionally having substituent(s), an acyl group or a heterocyclic group optionally having substituent(s)” represented by R^{1a}, those similar to the examples of R¹ can be used.

As the reactive derivative of compound (III), for example, a compound represented by the formula (IIIa):
R^{1a}-L (IIIa)
wherein L is a leaving group and R^{1a} is as defined above, or a salt thereof (hereinafter to be referred to as reactive derivative (IIIa)) can be used.

The leaving group represented by L includes, for example, a halogen atom (e.g., a chlorine atom, a bromine atom, an iodine atom, etc.), a substituted sulfonyloxy group (e.g., a C₁₋₆ alkylsulfonyloxy group such as methanesulfonyloxy, ethanesulfonyloxy, etc.; a C₆₋₁₄ arylsulfonyloxy group such as benzenesulfonyloxy, p-toluenesulfonyloxy, etc.; a C₇₋₁₆ aralkylsulfonyloxy group such as benzylsulfonyloxy, etc.; and the like), acyloxy (acetoxy, benzoyloxy, etc.), carbonates, trichioroacetimidic acid esters, oxalic acid esters, phosphorous acid esters (e.g., methyl phosphite, etc.), phosphoranes, an oxy group substituted with a heterocycle or an aryl group (succinimide, benzotriazole, quinoline, 4-nitrophenyl, etc.), a heterocycle (imidazole, etc.) and the like.

The reaction using the above-mentioned reactive derivative as an alkylating agent can be carried out by reacting compound (Ib) with the reactive derivative, usually in a solvent in the presence of a base. The solvent includes, for example, alcohols such as methanol, ethanol, propanol, etc.; ethers such as dimethoxyethane, dioxane, tetrahydrofuran, etc.; ketones such as acetone, etc.; nitrites such as acetonitrile, etc.; amides such as N,N-dimethylformamide, etc.; sulfoxides such as dimethyl sulfoxide, etc.; water and the like, which may be used in a suitable mixture. The base includes, for example, an organic base such as trimethylamine, triethylamine, N-methylmorpholine, pyridine, picoline, N,N-dimethylaniline, etc.; and an inorganic base such as potassium carbonate, sodium carbonate, potassium hydroxide, sodium hydroxide, etc. The amount of the base is, for example, about 1 to about 100 molar equivalents, preferably about 1 to about 10 molar equivalents, relative to 1 mol of the substrate.

The reactive derivative includes, for example, halides (e.g., chloride, bromide, iodide, etc.), sulfuric acid esters, or sulfonic acid esters (e.g., methanesulfonate, p-toluenesulfonate, benzenesulfonate, etc.) and the like, and particularly halides. The amount of the reactive derivative is, for example, about 1 to about 5 molar equivalents, preferably about 1 to about 3 molar equivalents, relative to 1 mol of the substrate.

If necessary, the reaction can be promoted by adding an additive. Such additive includes, for example, iodides such as sodium iodide, potassium iodide, etc. and the amount is about 0.1 to about 10 molar equivalents, preferably about 0.1 to about 5 molar equivalents, relative to 1 mol of the substrate.

The reaction temperature is usually about −10° C. to about 200° C., preferably about 0° C. to about 110° C., and the reaction time is usually about 0.5 to about 48 hr, preferably about 0.5 to about 16 hr.

The reaction using the above-mentioned reactive derivative as an acylating agent depends on the kind of reactive derivative or substrate, but it is usually carried out in a solvent. If necessary, a suitable base may be added to promote the reaction. The solvent includes, for example, hydrocarbons such as benzene, toluene, etc.; ethers such as diethyl ether, dioxane, tetrahydrofuran, etc.; esters such as ethyl acetate, etc.; halogenated hydrocarbons such as chloroform, dichloromethane, etc.; esters such as ethyl acetate, etc.; amides such as N,N-dimethylformamide, etc.; aromatic amines such as pyridine, etc.; water and the like, which may be used in a suitable mixture. In addition, the base includes, for example, alkali metal hydroxides such as sodium hydroxide, potassium hydroxide, etc.; hydrogen carbonates such as sodium hydrogen carbonate, potassium hydrogen carbonate, etc.; carbonates such as sodium carbonate, potassium carbonate, etc; acetates such as sodium acetate, etc.; tertiary amines such as trimethylamine, triethylamine, N-methylmorpholine, etc.; aromatic amines such as pyridine, picoline, N,N-dimethylaniline, etc.; and the like. The amount of the base is, for example, about 1 to about 100 molar equivalents, preferably about 1 to about 10 molar equivalents, relative to 1 mol of the substrate.

The acylating agent includes, for example, carboxylic acid, sulfonic acid, phosphoric acid, carbonic acid or a reactive derivative thereof (e.g., acid halide, acid anhydride, mixed acid anhydride, active ester, etc.), isocyanic acid ester, isothiocyanic acid ester and the like.

The amount of such acylating agent is usually about 1 to about 10 molar equivalents, preferably about 1 to about 3 molar equivalents, relative to 1 mol of the substrate. The reaction temperature is usually about −10° C. to about 150° C., preferably about 0° C. to about 100° C., and the reaction time is usually about 15 min to about 24 hr, preferably about 30 min to about 16 hr.

Compound (Ib) used as the starting compound in Method A can be produced by subjecting a compound represented by the formula (Ia) or a salt thereof (hereinafter to be referred to as compound (Ia)) obtained by Method B or Method C below to deacylation.

Such deacylation can be carried out according to a known method, for example, the methods described in Theodora W. Greene, Peter G. M. Wuts, “Protective Groups in Organic Synthesis, 3^{rd} Ed.,” (1999) Wiley-Interscience, and the like or a method analogous thereto. The reaction is usually carried out in the presence of an acid or a base, if necessary, in a solvent that does not adversely affect the reaction though it depends on the kinds of compound (Ia).

The acid is preferably mineral acids (e.g., hydrochloric acid, hydrobromic acid, sulfuric acid, etc.), carboxylic acids (e.g., acetic acid, trifluoroacetic acid, trichloroacetic acid, etc.), sulfonic acids (e.g., methanesulfonic acid, toluenesulfonic acid, etc.), Lewis acids (e.g., aluminum chloride, tin chloride, zinc bromide, etc.) and the like. If necessary, it may be used in a mixture of two or more. The amount of the acid varies depending on the kinds of the solvent and other reaction conditions, but it is usually about 0.1 mol or more, relative to 1 mol of compound (Ia), and the acid can be used as a solvent.

The base is preferably an inorganic base (alkali metal hydroxides such as sodium hydroxide, potassium hydroxide, etc.; alkali metal hydrogen carbonates such as sodium hydrogen carbonate, potassium hydrogen carbonate, etc.; alkali metal carbonates such as sodium carbonate, potassium carbonate, etc.; alkoxides such as sodium methoxide, sodium ethoxide, etc.; and the like), or an organic base (amines such as trimethylamine, triethylamine, diisopropylethylamine, etc.; cyclic amines such as pyridine, 4-dimethylaminopyridine, etc.; and the like) and the like, and preferably, sodium hydroxide, potassium hydroxide, sodium ethoxide and the like. The amount of the base varies depending on the kinds of the solvent and other reaction conditions, but is usually about 0.1 to about 10 mol, preferably about 0.1 to about 5 mol, relative to 1 mol of compound (Ia).

The solvent that does not adversely affect the reaction includes, for example, alcohols such as methanol, ethanol, propanol, 2-propanol, butanol, isobutanol, tert-butanol, etc.; aromatic hydrocarbons such as benzene, toluene, xylene, etc.; aliphatic hydrocarbons such as hexane, heptane, etc.; halogenated hydrocarbons such as dichloromethane, chloroform, etc.; ethers such as diethyl ether, diisopropyl ether, tert-butyl methyl ether, tetrahydrofuran, dioxane, dimethoxyethane, etc.; nitriles such as acetonitrile, etc.; esters such as ethyl acetate, etc.; carboxylic acids such as acetic acid, etc.; amides such as N,N-dimethylformamide, N,N-dimethylacetamide, etc.; sulfoxides such as dimethyl sulfoxide, etc.; water and the like. Such solvents may be used in a mixture of two or more at a suitable ratio.

The reaction temperature is for example, about −50° C. to about 200° C., preferably about 0° C. to about 100° C., and the reaction time varies depending on the kinds of compound (Ia), the reaction temperature and the like, and it is for example, about 0.5 to about 100 hr, preferably about 0.5 to about 24 hr.
[Method B] 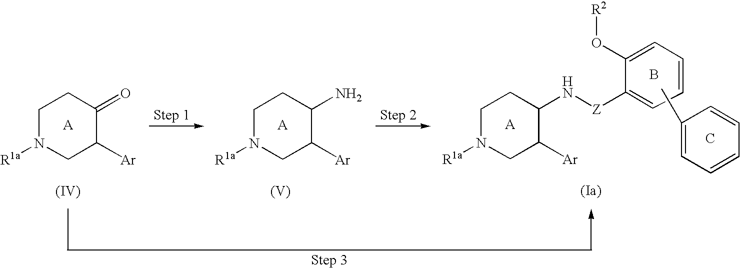
wherein each symbol is as defined above.

The compound (IV) to be used as a starting compound in this method can be produced according to the production method described in WO03/101964 and the like.

(Step 1)

In this step, a compound represented by the formula (IV) (hereinafter to be referred to as compound (IV)) is converted to an imine or oxime, and then subjected to a reduction to give a compound represented by the formula (V) (hereinafter to be referred to as amine compound (V)).

Compound (IV) can be converted to an imine or oxime by a known method and, for example, the reaction can be carried out in a solvent inert to the reaction using various amines.

As the amines, ammonias such as aqueous ammonia, ammonium chloride, ammonium acetate, etc.; hydroxylamines such as hydroxylamine, O-methylhydroxylamine, O-benzylhydroxylamine, etc.; organic amines such as benzylamine, aminodiphenylmethane, 1-phenylethylamine, etc.; and the like can be mentioned. These may be used in the form of a salt such as hydrochloride, sulfate and the like, or an aqueous solution thereof can also be used. The amount of the amine to be used is, for example, about 1 to about 50 mol, preferably about 1 to about 10 mol, per 1 mol of compound (IV).

The solvent inert to the reaction includes, for example, aromatic hydrocarbons such as toluene, xylene, etc.; aliphatic hydrocarbons such as heptane, hexane, etc.; halogenated hydrocarbons such as chloroform, dichloromethane, etc.; ethers such as diethyl ether, tetrahydrofuran, dioxane, etc.; alcohols such as methanol, ethanol, 2-propanol, butanol, benzyl alcohol, etc.; nitrites such as acetonitrile, etc.; N,N-dimethylformamide; dimethyl sulfoxide and the like. Such solvents may be used in a mixture at a suitable ratio.

Where necessary, the reaction can advantageously proceed by adding a catalyst. Such catalyst includes, for example, mineral acids (e.g., hydrochloric acid, hydrobromic acid, sulfuric acid, etc.), carboxylic acids (e.g., formic acid, acetic acid, propionic acid, trifluoroacetic acid, etc.), sulfonic acids (e.g., methanesulfonic acid, p-toluenesulfonic acid, etc.), Lewis acids (e.g., aluminum chloride, zinc chloride, zinc bromide, boron trifluoride, titanium chloride, etc.), acetates (e.g., sodium acetate, potassium acetate, etc.), molecular sieves (e.g., molecular sieves 3A, 4A, 5A, etc.), dehydrating agents (e.g., magnesium sulfate, etc.) and the like. The amount of the catalyst is, for example, about 0.01 to about 50 mol, preferably about 0.1 to about 10 mol, relative to 1 mol of compound (IV).

The reaction temperature is usually about 0° C. to about 200° C., preferably about 20° C. to about 150° C., and the reaction time is usually about 0.5 to about 48 hr, preferably about 0.5 to about 24 hr.

The imine or oxime can be converted to amine compound (V) by various reductions in a solvent inert to the reaction. Such reduction can be carried out by a method known per se, for example, a method using metal hydride or a method by catalytic hydrogenation.

The metal hydride includes, for example, sodium borohydride, lithium borohydride, zinc borohydride, sodium cyanoborohydride, sodium triacetoxyborohydride, lithium cyanoborohydride, dibutylaluminum hydride, aluminum hydride, lithium aluminum hydride, a borane complex (a borane-THF complex, catechol borane, etc.) and the like. The metal hydride includes preferably sodium borohydride, sodium cyanoborohydride, sodium triacetoxyborohydride, etc. The amount of the metal hydride is, for example, about 1 to about 50 mol, preferably about 1 to about 10 mol, relative to 1 mol of the imine or oxime.

The reduction by metal hydride is generally carried out in a solvent inert to the reaction. Such solvent includes, for example, aromatic hydrocarbons such as toluene, xylene, etc.; aliphatic hydrocarbons such as heptane, hexane, etc.; halogenated hydrocarbons such as chloroform, dichloromethane, etc.; ethers such as diethyl ether, tetrahydrofuran, dioxane, etc.; alcohols such as methanol, ethanol, 2-propanol, butanol, benzyl alcohol, etc.; nitrites such as acetonitrile, etc.; N,N-dimethylformamide; dimethyl sulfoxide and the like. Such solvents may be used in a mixture at a suitable ratio.

The reaction temperature is usually about −80° C. to about 80° C., preferably about −40° C. to about 40° C., and the reaction time is usually about 5 min to about 48 hr, preferably about 1 to about 24 hr.

The catalytic hydrogenation can be carried out under hydrogen atmosphere and in the presence of a catalyst. The catalyst to be used is preferably palladium compounds such as palladium carbon, palladium hydroxide, palladium oxide, etc.; nickel compounds such as Raney-nickel catalyst, etc.; platinum compounds such as platinum oxide, platinum carbon, etc.; rhodium compounds such as rhodium carbon, etc.; and the like, and the amount is about 0.001 to about 1 mol, preferably about 0.01 to about 0.5 mol, relative to 1 mol of the imine or oxime.

The catalytic hydrogenation proceeds usually in a solvent inert to the reaction. Such solvent includes, for example, alcohols such as methanol, ethanol, propanol, butanol, etc.; hydrocarbons such as benzene, toluene, xylene, etc.; halogenated hydrocarbons such as dichloromethane, chloroform, etc.; ethers such as diethyl ether, dioxane, tetrahydrofuran, etc.; esters such as ethyl acetate, etc.; amides such as N,N-dimethylformamide, etc.; carboxylic acids such as acetic acid, etc.; water, or a mixture thereof.

The hydrogen pressure under which the reaction proceeds is usually about 1 to about 50 atm, preferably about 1 to about 10 atm. The reaction temperature is usually about 0° C. to about 150° C., preferably about 20° C. to about 100° C., and the reaction time is usually about 5 min to about 72 hr, preferably about 0.5 to about 40 hr.

In the present step, amine compound (V) can also be produced directly from compound (IV) while carrying out the reactions of producing and of reducing imine or oxime at the same time, without isolating the intermediate imine or oxime. In this case, pH of the reaction mixture is preferably about 4 to about 5.

(Step 2)

In this step, amine compound (V) is subjected to an alkylation or reductive alkylation to give compound (Ia).

The alkylation can be carried out by a method known per se. For example, amine compound (V) is reacted with a compound represented by the formula (VI): 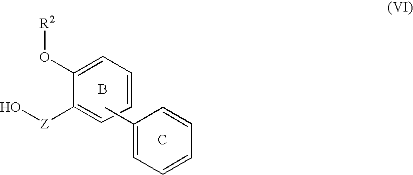
wherein each symbol is as defined above,
or a salt thereof (hereinafter to be referred to as compound (VI)) or a reactive derivative thereof, which is an alkylating agent.

As the reactive derivative of compound (VI), for example, a compound represented by the formula (VIa): 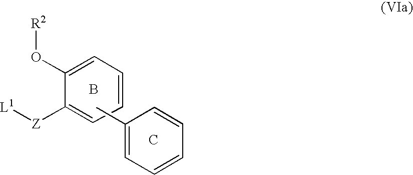
wherein L¹ is a leaving group and other symbols are as defined above,
or a salt thereof (hereinafter to be referred to as reactive derivative (VIa)) can be mentioned.

The leaving group represented by L¹ includes, for example, a halogen atom (e.g., a chlorine atom, a bromine atom, an iodine atom), a substituted sulfonyloxy group (e.g., a C₁₋₆ alkylsulfonyloxy group such as methanesulfonyloxy, ethanesulfonyloxy, etc.; a C₆₋₁₄ arylsulfonyloxy group such as benzenesulfonyloxy, p-toluenesulfonyloxy, etc.; a C₇₋₁₆ aralkylsulfonyloxy group such as benzylsulfonyloxy; a C₁₋₆ alkoxysulfonyloxy group such as methoxysulfonyloxy, etc.; and the like), and the like.

While the reaction using compound (VI) or the above-mentioned reactive derivative (VIa) as an alkylating agent varies depending on the kind of compound (VI) or reactive derivative (VIa) and amine compound (V), it generally includes reacting compound (VI) or reactive derivative (VIa) with amine compound (V) in a solvent in the presence of a base.

The solvent includes, for example, alcohols such as methanol, ethanol, propanol, etc.; ethers such as dimethoxyethane, dioxane, tetrahydrofuran, etc.; ketones such as acetone, etc.; nitriles such as acetonitrile, etc.; amides such as N,N-dimethylformamide, etc.; sulfoxides such as dimethyl sulfoxide, etc.; water and the like, which may be used in a suitable mixture.

The base includes, for example, an organic base such as trimethylamine, triethylamine, N-methylmorpholine, pyridine, picoline, N,N-dimethylaniline, etc.; and an inorganic base such as potassium carbonate, sodium carbonate, potassium hydroxide, sodium hydroxide, etc. The amount of the base is, for example, about 1 to about 100 mol, preferably about 1 to about 10 mol, relative to 1 mol of amine compound (V).

The reactive derivative (VIa) includes, for example, halides (e.g., chloride, bromide, iodide, etc.), sulfuric acid esters, or sulfonic acid esters (e.g., methanesulfonate, p-toluenesulfonate, benzenesulfonate, etc.) and the like, and particularly halides. The amount of compound (VI) or reactive derivative (VIa) is, for example, about 1 to about 5 mol, preferably about 1 to about 3 mol, relative to 1 mol of amine compound (V).

If necessary, the reaction can be promoted by adding an additive. Such additive includes, for example, iodides such as sodium iodide, potassium iodide, etc. and the amount is about 0.1 to about 10 mol, preferably about 0.1 to about 5 mol, relative to 1 mol of amine compound (V).

The reaction temperature is usually about −10° C. to about 200° C., preferably about 0° C. to about 110° C., and the reaction time is usually about 0.5 to about 48 hr, preferably about 0.5 to about 16 hr.

The reductive alkylation can be carried out by a method known per se. For example, amine compound (V) is reacted with a compound represented by the formula (VII): 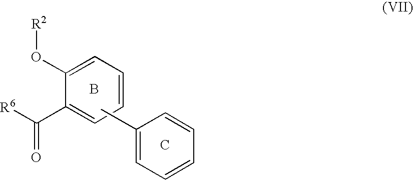
wherein R⁶ is a hydrogen atom or a C₁₋₆ alkyl group, and other symbols are as defined above, or a salt thereof (hereinafter to be referred to as compound (VII)) and the resulting imine or iminium ion is subjected to a reduction.

The reaction to produce imine or iminium ion and its reduction can be carried out according to the method described in Step 1.

In the present step, compound (Ia) can also be produced directly from amine compound (V) while carrying out the reactions of producing and of reducing imine or iminium ion at the same time, without isolating the intermediate imine or iminium ion. In this case, pH of the reaction mixture is preferably about 4 to about 5.

(Step 3)

In this reaction, compound (IV) is subjected to a reductive amination to give compound (Ia). This reaction can be carried out by a method known per se. For example, compound (IV) is reacted with a compound represented by the formula (VIII): 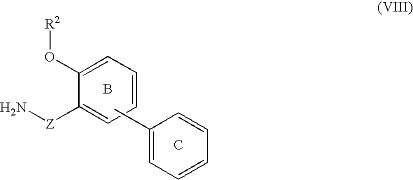
wherein each symbol is as defined above,
or a salt thereof (hereinafter to be referred to as compound (VIII)) and the resulting imine or iminium ion is subjected to a reduction.

The reaction to produce imine or iminium ion and its reduction can be carried out according to the method described in Step 1.

In the present step, compound (Ia) can also be produced directly from compound (IV) while carrying out the reactions of producing and of reducing imine or iminium ion at the same time, without isolating the intermediate imine or iminium ion. In this case, pH of the reaction mixture is preferably about 4 to about 5.
[Method C] 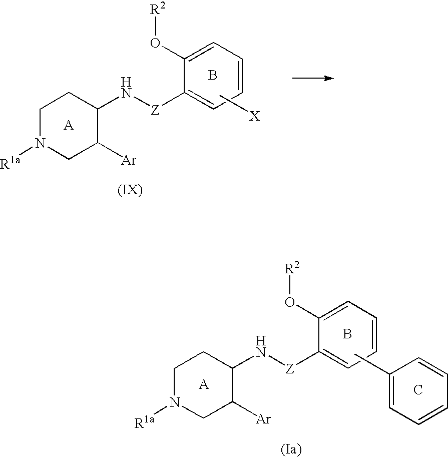
wherein X is a halogen atom such as iodine, bromine, chlorine and the like, or a substituted sulfonyloxy group such as a trifluoromethanesulfonyloxy group and the like, and other symbols are as defined above.

In this reaction, a compound represented by the formula (IX) or a salt thereof (hereinafter to be referred to as compound (IX)) is subjected to a coupling reaction with a compound represented by the formula (X): 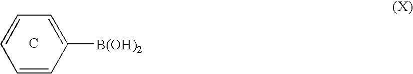
wherein each symbol is as defined above, or a salt thereof to give compound (Ia).

Compound (IX) to be used as a starting compound in this method can be produced according to a production method described in WO03/101964 and the like.

This step can be performed by a method known per se [e.g., Chemical Reviews, Vol. 95, p. 2457 (1995) and the like] and, for example, performed in the presence of a transition metal catalyst and a base in a solvent that does not adversely affect the reaction.

As the transition metal catalyst to be used, for example, palladium catalysts (palladium acetate, palladium chloride, tetrakis(triphenylphosphine)palladium, etc.), nickel catalysts (nickel chloride, etc.) and the like are used. Where necessary, ligands (triphenylphosphine, tri-t-butylphosphine, etc.) may be added or metal oxides (copper oxide, silver oxide, etc.) and the like may be used as cocatalysts. While the amount of the catalyst to be used varies depending on the kind of the catalyst, it is generally about 0.0001 to about 1 molar equivalent, preferably about 0.01 to about 0.5 molar equivalents, per 1 mol of compound (IX). The amount of the ligand to be used is generally about 0.0001 to about 4 molar equivalents, preferably about 0.01 to about 2 molar equivalents, per 1 mol of compound (IX), and the amount of the cocatalyst to be used is about 0.0001 to about 4 molar equivalents, preferably about 0.01 to about 2 molar equivalents, per 1 mol of compound (IX).

As the base to be used, for example, organic amines (trimethylamine, triethylamine, diisopropylamine, N-methylmorpholine, 1,8-diazabicyclo[5.4.0]undec-7-ene, pyridine, N,N-dimethylaniline, etc.), alkali metal salts (sodium hydrogen carbonate, potassium hydrogen carbonate, sodium carbonate, potassium carbonate, cesium carbonate, sodium phosphate, potassium phosphate, sodium hydroxide, potassium hydroxide, etc.), metal hydrides (potassium hydride, sodium hydride, etc.), alkali metal alkoxides (sodium methoxide, sodium ethoxide, sodium t-butoxide, potassium t-butoxide, etc.), alkali disilazides (lithium disilazide, sodium disilazide, potassium disilazide, etc.) and the like can be mentioned. Of these, alkali metal salts such as potassium carbonate, cesium carbonate, sodium phosphate, potassium phosphate and the like; alkali metal alkoxides such as sodium t-butoxide, potassium t-butoxide and the like; organic amines such as triethylamine, diisopropylamine and the like; and the like are preferable. The amount of the base to be used is about 0.1 to about 10 molar equivalents, preferably about 1 to about 5 molar equivalents, per 1 mol of compound (IX).

The solvent to be used may be any as long as it does not adversely affect the reaction and, for example, hydrocarbons (benzene, toluene, xylene, etc.), halogenated hydrocarbons (chloroform, 1,2-dichloroethane, etc.), nitriles (acetonitrile, etc.), ethers (dimethoxyethane, tetrahydrofuran, etc.), alcohols (methanol, ethanol, etc.), aprotic polar solvents (N,N-dimethylformamide, dimethyl sulfoxide, hexamethylphosphoramide, etc.), water or a mixture thereof can be used. The reaction temperature is generally about −10° C. to about 200° C., preferably about 0° C. to about 150° C., and the reaction time is generally about 0.5 to about 48 hr, preferably about 0.5 to about 16 hr.
[Method D] 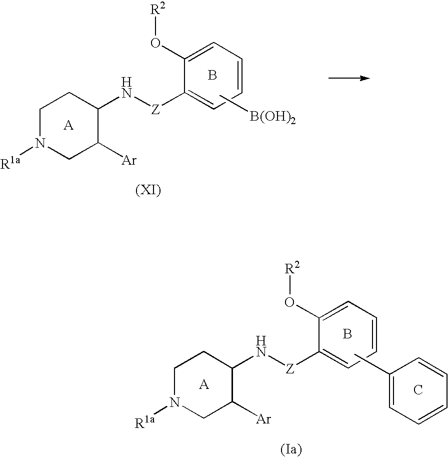
wherein each symbol is as defined above.

In this reaction, a compound represented by the formula (XI) or a salt thereof (hereinafter to be referred to as compound (XI)) is subjected to a coupling reaction with a compound represented by the formula (XII): 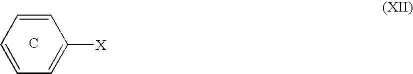
wherein each symbol is as defined above, or a salt thereof to give compound (Ia), which can be performed by a method similar to the method described in Method C.

Compound (XI) to be used as a starting compound in this method can be produced according to the production method described in WO03/101964 and the like.

In the above-mentioned method, by using, as a starting compound, an optically active compound represented by the formula (Va): 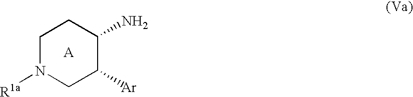
wherein each symbol is as defined above (hereinafter to be referred to as compound (Va)) instead of amine compound (V), optically active compound (I) can be produced. Compound (Va) to be used as a starting compound in this method can be produced according to the production method described in WO03/101964 and the like.

When compound (I) is obtained as a free compound in the above-mentioned method, a salt with for example, inorganic acids (e.g., hydrochloric acid, sulfuric acid, hydrobromic acid, etc.), organic acids (e.g., methanesulfonic acid, benzenesulfonic acid, toluenesulfonic acid, oxalic acid, fumaric acid, maleic acid, tartaric acid, etc.), inorganic bases (e.g., alkali metals such as sodium, potassium, etc.; alkaline earth metals such as calcium, magnesium, etc.; aluminum, ammonium, etc.), or organic bases (e.g., trimethylamine, triethylamine, pyridine, picoline, ethanolamine, diethanolamine, triethanolamine, dicyclohexylamine, N,N′-dibenzylethylenediamine, etc.) and the like can be produced in a routine manner. When compound (I) is obtained in the form of a salt, the compound can be converted to a free compound or another salt in a routine manner.

In addition, when the starting compound forms a salt in each of the above-mentioned reactions, the compound may-be used as a salt. Such salt includes, for example, those exemplified as a salt of compound (I).

Compound (I) thus produced by such method can be isolated and purified by a typical separation means such as recrystallization, distillation, chromatography, etc.

When compound (I) contains an optical isomer, a stereoisomer, a regioisomer or a rotamer, these are also encompassed in compound (I), and can be obtained as a single product according to synthesis and separation methods known per se (e.g., concentration, solvent extraction, column chromatography, recrystallization, etc.). For example, when compound (I) has an optical isomer, an optical isomer resolved from this compound is also encompassed in compound (I).

The optical isomer can be produced by a method known per se. To be specific, an optically active synthetic intermediate is used, or the final racemate product is subjected to optical resolution according to a conventional method to give an optical isomer.

The method of optical resolution may be a method known per se, such as a fractional recrystallization method, a chiral column method, a diastereomer method, etc.

1) Fractional Recrystallization Method

A method wherein a salt of a racemate with an optically active compound (e.g., (+)-mandelic acid, (−)-mandelic acid, (+)-tartaric acid, (−)-tartaric acid, (+)-1-phenethylamine, (−)-1-phenethylamine, cinchonine, (−)-cinchonidine, brucine, etc.) is formed, which is separated by a fractional recrystallization method, and if desired, a free optical isomer is obtained by a neutralization step.

2) Chiral Column Method

A method wherein a racemate or a salt thereof is applied to a column for separation of an optical isomer (a chiral column) to allow separation. In the case of a liquid chromatography, for example, a mixture of the optical isomers is applied to a chiral column such as ENANTIO-OVM (manufactured by Tosoh Corporation), CHIRAL series (manufactured by Daicel Chemical Industries, Ltd.) and the like, and developed with water, various buffers (e.g., phosphate buffer, etc.) and organic solvents (e.g., ethanol, methanol, isopropanol, acetonitrile, trifluoroacetic acid, diethylamine, etc.) solely or in admixture to separate the optical isomer. In the case of a gas chromatography, for example, a chiral column such as CP-Chirasil-DeX CB (manufactured by GL Sciences Inc.) and the like is used to allow separation.

3) Diastereomer Method

A method wherein a racemic mixture is prepared into a diastereomeric mixture by chemical reaction with an optically active reagent, which is made into a single substance by a typical separation means (e.g., a fractional recrystallization method, a chromatography method, etc.) and the like, and is subjected to a chemical treatment such as hydrolysis and the like to separate an optically active reagent moiety, whereby an optical isomer is obtained. For example, when compound (I) contains hydroxy, or primary or secondary amino in a molecule, the compound and an optically active organic acid (MTPA [α-methoxy-α-(trifluoromethyl)phenylacetic acid], (−)-menthoxyacetic acid, etc.) and the like are subjected to condensation reaction to give diastereomers in the ester form or in the amide form, respectively. When compound (I) has a carboxylic acid group, this compound and an optically active amine or an alcohol reagent are subjected to condensation reaction to give diastereomers in the amide form or in the ester form, respectively. The separated diastereomer is converted to an optical isomer of the original compound by acid hydrolysis or base hydrolysis.

Compound (I) may be in the form of a crystal.

The crystal of compound (I) can be produced by crystallization of compound (I) by a crystallization method known per se.

Examples of the crystallization method include a method of crystallization from a solution, a method of crystallization from vapor, a method of crystallization from the melts and the like.

The “crystallization from a solution” is typically a method of shifting a non-saturated state to supersaturated state by varying factors involved in solubility of compounds (solvent composition, pH, temperature, ionic strength, redox state, etc.) or the amount of solvent. To be specific, for example, a concentration method, a cooling method, a reaction method (a diffusion method, an electrolysis method), a hydrothermal growth method, a flux method and the like can be mentioned. Examples of the solvent to be used include aromatic hydrocarbons (e.g., benzene, toluene, xylene, etc.), halogenated hydrocarbons (e.g., dichloromethane, chloroform, etc.), saturated hydrocarbons (e.g., hexane, heptane, cyclohexane, etc.), ethers (e.g., diethyl ether, diisopropyl ether, tetrahydrofuran, dioxane, etc.), nitrites (e.g., acetonitrile, etc.), ketones (e.g., acetone, etc.), sulfoxides (e.g., dimethyl sulfoxide, etc.), acid amides (e.g., N,N-dimethylformamide, etc.), esters (e.g., ethyl acetate, etc.), alcohols (e.g., methanol, ethanol, isopropyl alcohol, etc.), water and the like. These solvents are used alone or in a combination of two or more at a suitable ratio (e.g., 1:1 to 1:100 (a volume ratio)). Where necessary, a seed crystal can be used.

The “crystallization from vapor” is, for example, a vaporization method (a sealed tube method, a gas stream method), a gas phase reaction method, a chemical transportation method and the like.

The “crystallization from the melts” is, for example, a normal freezing method (a Czockralski method, a temperature gradient method and a Bridgman method, etc.), a zone melting method (a zone leveling method and a floating zone method, etc.), a special growth method (a VLS method and a liquid phase epitaxy method, etc.) and the like.

Preferable examples of the crystallization method include a method of dissolving compound (I) in a suitable solvent (e.g., alcohols such as methanol, ethanol, etc., and the like) at a temperature of 20 to 120° C., and cooling the resulting solution to a temperature not higher than the temperature of dissolution (e.g., 0 to 50° C., preferably 0 to 20° C.) and the like.

The thus obtained crystals of the present invention can be isolated, for example, by filtration and the like.

As the analysis method of the obtained crystals, crystal analysis by powder X-ray diffraction is generally employed. As a method for determining the crystal orientation, mechanical methods, optical methods and the like can also be mentioned.

The crystal of compound (I) obtained by the above-mentioned production method (hereinafter to be abbreviated as “crystal of the present invention”) has high purity and high quality, shows low hygroscopicity, is not denatured even after a long-term preservation under normal conditions, and is extremely superior in stability. In addition, it is superior in biological properties (pharmacokinetics (absorption, distribution, metabolism, excretion) and efficacy expression, etc.), and therefore, extremely useful as a medicament.

In the present specification, the specific rotation ([α]_{D}) means, for example, a specific rotation measured using a polarimeter (JASCO, P-1030 polarimeter (No. AP-2)) and the like.

In the present specification, the melting point means that measured using, for example, a micromelting point apparatus (Yanako, MP-500D) or a DSC (differential scanning calorimetry) device (SEIKO, EXSTAR6000) and the like.

In the present specification, the peak by a powder X-ray diffraction means that measured using, for example, RINT Ultima⁺ 2100 (Rigaku Corporation), etc. with a Cu—K_{α} ray as a ray source.

In general, the melting points and the peak by a powder X-ray diffraction vary depending on the measurement apparatuses, the measurement conditions and the like. The crystal in the present specification may show different values from the melting point or the peak by a powder X-ray diffraction described in the present specification vary depending on the measurement apparatuses, as long as they are within each of a general error range.

The compound of the present invention has excellent antagonistic action for a tachykinin receptor, particularly substance P receptor antagonistic action including inhibitory action for the increased permeability of blood vessel of a trachea induced by capsaicin, neurokinin A receptor antagonistic action. The compound of the present invention has low toxicity and thus it is safe.

Accordingly, the compound of the present invention having excellent antagonistic action for substance P receptors and neurokinin A receptors, etc. can be used as a safe medicine for preventing and treating the following diseases related to substance P in mammals (e.g., mice, rats, hamsters, rabbits, cats, dogs, bovines, sheep, monkeys, humans, etc.).
(1) Lower urinary tract symptoms [for example, dysuria such as an overactive bladder, lower urinary tract symptoms associated with benign prostatic hyperplasia, a pelvic visceral pain, lower urinary tract symptoms associated with chronic prostatitis, lower urinary tract symptoms associated with interstitial cystitis and the like]
(2) Digestive organ diseases [for example, an irritable bowel syndrome, inflammatory bowel disease, ulcerative colitis, Crohn's disease, diseases caused by a spiral urease-positive gram-negative bacterium (e.g., Helicobacter pylori, etc.) (e.g., gastritis, gastric ulcer, etc.), gastric cancer, postgastrostomy disorder, indigestion, esophageal ulcer, pancreatitis, polyp of the colon, cholelithiasis, hemorrhoids, peptic ulcer, situational ileitis, vomiting, nausea, etc.]
(3) Inflammatory or allergic diseases [for example, allergic rhinitis, conjunctivitis, gastrointestinal allergy, pollinosis, anaphylaxis, dermatitis, herpes, psoriasis, bronchitis, expectoration, retinopathy, postoperative and posttraumatic inflammation, puffiness, pharyngitis, cystitis, meningitidis, inflammatory ophthalmic diseases, etc.]
(4) Osteoarthropathy diseases [for example, rheumatoid arthritis (chronic rheumatoid arthritis), arthritis deformans, rheumatoid myelitis, osteoporosis, abnormal growth of cells, bone fracture, bone refracture, osteomalacia, osteopenia, Paget's disease of bone, rigid myelitis, articular tissue destruction by gonarthrosis deformans and similar diseases thereto, etc.]
(5) Respiratory diseases [for example, cold syndrome, pneumonia, asthma, pulmonary hypertension, pulmonary thrombi/pulmonary obliteration, pulmonary sarcoidosis, pulmonary tuberculosis, interstitial pneumonia, silicosis, adult respiratory distress syndrome, chronic obstructive pulmonary diseases, cough, etc.]
(6) Infectious diseases [HIV infectious diseases, virus infectious diseases due to cytomegalo virus, influenza virus, herpes virus and the like, rickettsia infectious diseases, bacterial infectious diseases, sexually-transmitted diseases, carinii pneumonia, helicobacter pylori infectious disease, systemic fungal infectious diseases, tuberculosis, invasive staphylococcal infectious diseases, acute viral encephalitis, acute bacterial meningitidis, AIDS encephalitis, septicemia, sepsis, sepsis gravis, septic shock, endotoxin shock, toxic shock syndromes, etc.]
(7) Cancers [for example, primary, metastatic or recurrent breast cancer, prostatic cancer, pancreatic cancer, gastric cancer, lung cancer, colorectal cancer (colon cancer, rectal cancer, anal cancer), esophagus cancer, duodenal cancer, head and neck cancer (tongue cancer, pharynx cancer, larynx cancer), brain tumor, neurinoma, non-small cell lung cancer, small cell lung cancer, hepatic cancer, renal cancer, colic cancer, uterine cancer (cancer of the uterine body, uterine cervical cancer), ovarian cancer, bladder cancer, skin cancer, hemangioma, malignant lymphoma, malignant melanoma, thyroid cancer, bone tumor, angiofibroma, retinosarcoma, penis cancer, pediatric solid cancer, Kaposi's sarcoma, Kaposi's sarcoma caused by AIDS, tumor of the maxillary sinus, fibrous histiocytoma, smooth muscle sarcoma, rhabdomyosarcoma, liposarcoma, fibroid tumors of the uterus, osteoblastoma, osteosarcoma, chondrosarcoma, carcinomatous mesothelial tumor, tumors such as leukemia, Hodgkin's disease, etc.]
(8) Central nerve diseases [for example, neurodegenerative diseases (e.g., Alzheimer's disease, Down's disease, Parkinson's disease, Creutzfeldt-Jakob's disease, amyotrophic lateral sclerosis (ALS), Huntington chorea, diabetic neuropathy, multiple sclerosis, etc.), mental diseases (e.g., schizophrenia, depression, mania, anxiety neurosis, obsessive-compulsive neurosis, panic disorder, epilepsy, alcohol dependence, an anxiety symptom, anxious mental state, etc.), central and peripheral nerve disorders (e.g., head trauma, spinal cord injury, brain edema, disorders of sensory function, abnormality of sensory function, disorders of autonomic nervous function and abnormality of autonomic nervous function, whiplash injury, etc.), memory disorders (e.g., senile dementia, amnesia, cerebrovascular dementia, etc.), cerebrovascular disorders (e.g., disorders and aftereffect and/or complication from intracerebral hemorrhage, brain infarction, etc., asymptomatic cerebro-vascular accident, transient cerebral ischemic attack, hypertensive encephalopathia, blood-brain barrier disorder, etc.), recurrence and aftereffect of cerebro-vascular accident (e.g., neural symptoms, mental symptoms, subjective symptoms, disorders of daily living activities, etc.), post-cerebrovascular occlusion central hypofunction, disorder or abnormality of autoregulation of cerebral circulation and/or renal circulation, sleep disorder (insomnia), etc.]
(9) Circulatory diseases [for example, acute coronary artery syndromes (e.g., acute cardiac infarction, unstable angina, etc.), peripheral arterial obstruction, Raynaud's disease, Buerger disease, restenosis after coronary-artery intervention (percutaneous transluminal coronary angioplasty (PTCA), directional coronary atherectomy (DCA), stenting, etc.), restenosis after coronary-artery bypass operation, restenosis after intervention (angioplasty, atherectomy, stenting, etc.) or bypass operation in other peripheral artery, ischemic cardiac diseases (e.g., cardiac infarction, angina, etc.), myocarditis, intermittent claudication, lacunar infarction, arteriosclerosis (e.g., atherosclerosis, etc.), cardiac failure (acute cardiac failure, chronic cardiac failure accompanied by congestion), arrhythmia, progress of atherosclerotic plaque, thrombosis, hypertension, hypertensive tinnitus, hypotension, etc.]
(10) Pains [e.g., migraine, neuralgia, pelvic visceral pain including cystalgia, etc.]
(11) Autoimmune diseases [for example, collagen disease, systemic lupus erythematosus, scleroderma, polyarteritis, myasthenia gravis, multiple sclerosis, Sjogren's syndrome, Behcet's disease, etc.]
(12) Hepatic diseases [e.g., hepatitis (including chronic hepatitis), cirrhosis, interstitial hepatic diseases, etc.]
(13) Pancreatic diseases [e.g., pancreatitis (including chronic pancreatitis), etc.]
(14) Renal diseases [e.g., nephritis, glomerulonephritis, glomerulosclerosis, renal failure, thrombotic microangiopathy, dialysis complications, organ disorders including nephropathia by radiation, diabetic nephropathia, etc.]
(15) Metabolic diseases [e.g., diabetic diseases (insulin-dependent diabetes, diabetic complications, diabetic retinopathy, diabetic microangiopathy, diabetic neuropathy, etc.), glucose tolerance abnormality, obesity, benign prostatic hyperplasia, sexual dysfunction, etc.]
(16) Endocrine diseases [e.g., Addison's disease, Cushing's syndrome, melanocytoma, primary aldosteronism, etc.]
(17) Other diseases
(a) Transplant rejection [e.g., posttransplantational rejection, posttransplantational polycythemia, hypertension, organ disorder and/or vascular hypertrophy, graft-versus-host disease, etc.]
(b) Abnormality in characteristic of blood and/or blood components [e.g., enhancement in platelet aggregation, abnormality of erythrocyte deformability, enhancement in leukocyte adhesiveness, increase in blood viscosity, polycythemia, vascular peliosis, autoimmune hemolytic anemia, disseminated intravascular coagulation syndrome (DIC), multiple myelopathy, etc.]
(c) Gynecologic diseases [e.g., climacteric disorder, gestational toxicosis, endometriosis, hysteromyoma, ovarian disease, mammary disease, etc.]
(d) Dermatic diseases [e.g., keloid, angioma, psoriasis, pruritus, etc.]
(e) Ophthalmic diseases [e.g., glaucoma, ocular hypertension disease, etc.]
(f) Otolaryngological diseases [e.g., Menuel syndrome, tinnitus, gustation disorder, dizziness, disequilibrium, dysphagia, etc.]
(g) Diseases due to environmental and/or occupational factors (e.g., radiation disorder, disorders by ultraviolet ray-infrared ray-laser ray, altitude sickness, etc.)
(h) Ataxia
(i) Chronic fatigue syndrome

Of these diseases, the compounds of the present invention are particularly useful as tachykinin receptor antagonists, an agent for improving lower urinary tract symptoms such as urinary frequency, urinary incontinence and the like and a therapeutic drug for the above-mentioned lower urinary tract symptoms.

Pharmaceutical preparations comprising compound of the present invention may be in any solid forms of powders, granules, tablets, capsules, suppositories, etc., and in any liquid forms of syrups, emulsions, injections, suspensions, etc.

The pharmaceutical preparations comprising compound of the present invention can be produced by any conventional methods, for example, blending, kneading, granulation, tabletting, coating, sterilization, emulsification, etc., in accordance with the forms of the preparations to be produced. For the production of such pharmaceutical preparations, for example, each of the items in General Rules for Preparations in the Japanese Pharmacopoeia, can be made reference to. In addition, the pharmaceutical preparations of the present invention may be formulated into a sustained release preparation containing active ingredients and biodegradable polymer compounds. The sustained release preparation can be produced according to the method described in JP-A-9-263545.

In the pharmaceutical preparations of the present invention, the content of the compound of the present invention or a salt thereof varies depending on the forms of the preparations, but is generally in a range of about 0.01 to 100% by weight, preferably about 0.1 to 50% by weight, more preferably 0.5 to 20% by weight, relative to the total weight of each preparation.

When the compound of the present invention is used in the above-mentioned pharmaceutical preparations, it may be used alone, or in admixture with a suitable, pharmaceutically acceptable carrier, for example, excipients (e.g., starch, lactose, sucrose, calcium carbonate, calcium phosphate, etc.), binders (e.g., starch, arabic gum, carboxymethyl cellulose, hydroxypropyl cellulose, crystalline cellulose, alginic acid, gelatin, polyvinyl pyrrolidone, etc.), lubricants (e.g., stearic acid, magnesium stearate, calcium stearate, talc, etc.), disintegrants (e.g., calcium carboxymethylcellulose, talc, etc.), diluents (e.g., water for injection, physiological saline, etc.) and if desired, with the additives (e.g., a stabilizer, a preservative, a colorant, a fragrance, a dissolution aid, an emulsifier, a buffer, an isotonic agent, etc.) and the like, by ordinary methods. It can be formulated into the solid preparations such as powders, fine granules, granules, tablets, capsules, etc., or into the liquid preparations such as injections, etc., and can be administered non-parenterally or parenterally.

The dose of the pharmaceutical preparation of the present invention varies depending on the kinds of the compound of the present invention or a pharmaceutically acceptable salt thereof, the administration route, the condition and the age of patients, etc. For example, the dose for oral administration of the pharmaceutical preparation to an adult patient suffering from dysuria is generally from about 0.005 to 50 mg/kg body/day, preferably from about 0.05 to 10 mg/kg body/day, more preferably from about 0.2 to 4 mg/kg body/day, in terms of the compound of the present invention, which may be administered once a day or in two or three divided portions a day.

The dose when the pharmaceutical composition of the present invention is a sustained release preparation varies depending on the kinds and the content of compound (I), the formulation, the duration time of drug release, the animals to be administered (e.g., mammals such as humans, rats, mice, cats, dogs, rabbits, bovines, pigs, etc.), and the purpose of administration. For example, when it is applied by parenteral administration, preferably about 0.1 to about 100 mg of compound (I) is released from the preparation for 1 week.

The compound of the present invention can be used in a mixture or combination with other pharmaceutically active ingredients at a suitable ratio.

Combination of the compound of the present invention with other pharmaceutically active ingredients can give the following excellent effects:
(1) a dose can be reduced as compared with separate administration of the compound of the present invention or other pharmaceutically active ingredients. More specifically, when the compound of the present invention is combined with anticholinergic agents or NK-2 receptor antagonists, the dose can be reduced as compared with separate administration of anticholinergic agents or NK-2 receptor antagonists, and therefore, side effects such as dry mouth can be reduced;
(2) according to symptoms of patient (mild symptoms, severe symptoms, etc.), a drug to be combined with the compound of the present invention can be selected;
(3) by choosing other pharmaceutically active ingredients which have different mechanism of action from that of the compound of the present invention, the therapeutic period can be designed longer;
(4) by choosing other pharmaceutically active ingredients which have different mechanism of action from that of the compound of the present invention, continuation of therapeutic effects can be obtained; and
(5) by combining the compound of the present invention and other pharmaceutically active ingredients, excellent effects such as synergic effects can be obtained.

A drug which is mixed or combined with the compound of the present invention (hereinafter, briefly referred to as combination drugs) includes the following:

(1) Agent for Treating Diabetes

Insulin preparations (e.g., animal insulin preparations extracted from the bovine or swine pancreas; human insulin preparations synthesized by a genetic engineering technique using Escherichia coli or a yeast; insulin zinc; protamine zinc insulin; a fragment or a derivative of insulin (e.g., INS-1, etc.), insulin sensitizers (e.g., pioglitazone hydrochloride, troglitazone, rosiglitazone or its maleate, JTT-501, MCC-555, YM-440, GI-262570, KRP-297, FK-614, CS-011, etc.), α-glucosidase inhibitors (e.g., voglibose, acarbose, miglitol, emiglitate, etc.), biguanides (e.g., phenformin, metformin, buformin, etc.), sulfonylureas (e.g., tolbutamide, glibenclamide, gliclazide, chlorpropamide, tolazamide, acetohexamide, glyclopyramide, glimepiride, etc.) and other insulin secretagogues (e.g., repaglinide, senaglinide, mitiglinide or its calcium salt hydrate, GLP-1, nateglinide, etc.), dipeptidylpeptidase IV inhibitors (e.g., NVP-DPP-278, PT-100, P32/98, etc.), β₃ agonists (e.g., CL-316243, SR-58611-A, UL-TG-307, AJ-9677, AZ40140, etc.), amylin agonists (e.g., pramlintide, etc.), phosphotyrosine phosphatase inhibitors (e.g., vanadic acid, etc.), gluconeogenesis inhibitors (e.g., glycogen phosphorylase inhibitors, glucose-6-phosphatase inhibitors, glucagon antagonists, etc.), SGLT (sodium-glucose cotransporter) inhibitors (e.g., T-1095, etc.) and the like.

(2) Agent for Treating Diabetic Complications

Aldose reductase inhibitors (e.g., tolrestat, epalrestat, zenarestat, zopolrestat, fidarestat (SNK-860), minalrestat (ARI-509), CT-112, etc.), neurotrophic factors (e.g., NGF, NT-3, etc.), AGE inhibitors (e.g., ALT-945, pimagedine, pyratoxathine, N-phenacylthiazolium bromide (ALT-766), EXO-226, etc.), active oxygen scavengers (e.g., thioctic acid, etc.), cerebral vasodilators (e.g., tiapuride, etc.) and the like.

(3) Antihyperlipidemic Agent

Statin compounds inhibiting cholesterol synthesis (e.g., pravastatin, simvastatin, lovastatin, atorvastatin, fluvastatin, cerivastatin or their salts (e.g., sodium salt, etc.), etc.), squalene synthase inhibitors or fibrate compounds having triglyceride lowering action (e.g., bezafibrate, clofibrate, simfibrate, clinofibrate, etc.) and the like.

(4) Hypotensive Agent

Angiotensin converting enzyme inhibitors (e.g., captopril, enalapril, delapril, etc.), angiotensin II antagonists (e.g., losartan, candesartan cilexetil, etc.), calcium antagonists (e.g., manidipine, nifedipine, amlodipine, efonidipine, nicardipine, etc.), clonidine, and the like. (

5) Antiobesity Agent

Antiobesity drugs acting on the central nervous system (e.g. dexfenfluramine, fenfluramine, phentermine, sibutramine, anfepramone, dexamphetamine, mazindol, phenylpropanolamine, clobenzorex, etc.), pancreatic lipase inhibitors (e.g. orlistat, etc.), β₃ agonists (e.g. CL-316243, SR-58611-A, UL-TG-307, AJ-9677, AZ40140, etc.), anorectic peptides (e.g. leptin, CNTF (Ciliary Neurotrophic Factor), etc.), cholecystokinin agonists (e.g. lintitript, FPL-15849, etc.), cannabinoid CB1 receptor antagonists (e.g., rimonabant), and the like.

(6) Diuretic Agent

Xanthine derivatives (e.g., theobromine sodium salicylate, theobromine calcium salicylate, etc.), thiazide preparations (e.g., ethiazide, cyclopenthiazide, trichlormethiazide, hydrochlorothiazide, hydroflumethiazide, benzylhydrochlorothiazide, penflutizide, polythiazide, methyclothiazide, etc.), antialdosterone preparations (e.g., spironolactone, triamterene, etc.), carbonic anhydrase inhibitors (e.g., acetazolamide, etc.), chlorobenzenesulfonamide preparations (e.g., chlorthalidone, mefruside, indapamide, etc.), azosemide, isosorbide, ethacrynic acid, piretanide, bumetanide, furosemide, etc.

(7) Chemotherapeutic Agent

Alkylating agents (e.g., cyclophosphamide, ifosamide, etc.), metabolic antagonists (e.g., methotrexate, 5-fluorouracil, etc.), antitumor antibiotics (e.g., mitomycin, adriamycin, etc.), plant-derived antitumor agents (e.g., vincristine, vindesine, taxol, etc.), cisplatin, carboplatin, etoposide, etc. Among these, 5-fluorouracil derivatives such as Furtulon and Neo-Furtulon are preferred.

(8) Immunotherapeutic Agent

Microorganism- or bacterium-derived components (e.g., muramyl dipeptide derivatives, Picibanil, etc.), immunopotentiator polysaccharides (e.g., lentinan, schizophyllan, krestin, etc.), genetically engineered cytokines (e.g., interferons, interleukins (IL), etc.), colony stimulating factors (e.g., granulocyte colony stimulating factor, erythropoietin, etc.) and the like. Among these, IL-1, IL-2, IL-12, etc. are preferred.

(9) Therapeutic Agent Recognized to Ameliorate Cachexia in Animal Models or Clinical Practice

Progesterone derivatives (e.g., megestrol acetate) [Journal of Clinical Oncology, vol. 12, pp. 213-225, 1994], metoclopramide pharmaceuticals, tetrahydrocannabinol pharmaceuticals (the above references are applied to both), fat metabolism ameliorating agents (e.g., eicosapentanoic acid) [British Journal of Cancer, vol. 68, pp. 314-318, 1993], growth hormones, IGF-1, and antibodies to the cachexia-inducing factors such as TNF-α, LIF, IL-6 and oncostatin M.

(10) Antiinflammatory Agent

Steroids (e.g., dexamethasone, etc.), sodium hyaluronate, cyclooxygenase inhibitors (e.g., indomethacin, ketoprofen, loxoprofen, meloxicam, ampiroxicam, celecoxib, rofecoxib, etc.) and the like.

(11) Miscellaneous

Glycosylation inhibitors (e.g., ALT-711, etc.), nerve regeneration promoting drugs (e.g., Y-128, VX853, prosaptide, etc.), drugs acting on the central nervous system (e.g., antidepressants such as desipramine, amitriptyline, imipramine, fluoxetine, paroxetine, doxepin, duloxetine, venlafaxine, etc.), anticonvulsants (e.g., lamotrigine, carbamazepine, gabapentin), antiarrhythmic drugs (e.g., mexiletine), acetylcholine receptor ligands (e.g., ABT-594), endothelin receptor antagonists (e.g., ABT-627), monoamine uptake inhibitors (e.g., tramadol), indoleamine uptake inhibitors (e.g., fluoxetine, paroxetine), narcotic analgesics (e.g., morphine), nonnarcotic analgesics (e.g., buprenorphine, axomadol), GABA receptor agonists,. GABA uptake inhibitors (e.g., tiagabine), α₂ receptor agonists (e.g., clonidine), local analgesics (e.g., capsaicin), protein kinase C inhibitors (e.g., LY-333531), antianxiety drugs (e.g., benzodiazepines), phosphodiesterase inhibitors (e.g., sildenafil), dopamine receptor agonists (e.g., apomorphine), dopamine receptor antagonists (e.g., haloperidol), serotonin receptor agonists (e.g., tandospirone citrate, sumatryptan, tegaserod), serotonin receptor antagonists (e.g., cyproheptadine hydrochloride, ondansetron), serotonin uptake inhibitors (e.g., fluvoxamine maleate, fluoxetine, paroxetine), sleep inducing drugs (e.g., triazolam, zolpidem), hypnotics (e.g., ramelteon), anticholinergic agents, α₁ receptor blocking agents (e.g., tamsulosin, urapidil, naftopidil, silodosin), agents for treating overactive bladder (e.g., flavoxate hydrochloride), muscle relaxants (e.g., baclofen, etc.), potassium channel openers (e.g., nicorandil), calcium channel blocking agents (e.g., nifedipine), chloride channel openers (activators) (e.g., lubiprostone), agents for preventing and/or treating Alzheimer's disease (e.g., donepezil, rivastigmine, galanthamine), agents for treating Parkinson's disease (e.g., L-dopa), agents for preventing and/or treating multiple sclerosis (e.g., interferon β-1a), histamine H₁ receptor inhibitors (e.g., promethazine hydrochloride), proton pump inhibitors (e.g., lansoprazole, omeprazole), antithrombotic agents (e.g., aspirin, cilostazol), NK-2 receptor antagonists, NK-3 receptor antagonists (e.g., talnetant), agents of treating HIV infection (saquinavir, zidovudine, lamivudine, nevirapine), agents of treating chronic obstructive pulmonary diseases (salmeterol, thiotropium bromide, cilomilast), diuretic agents (e.g., furosemide), antidiuretic agents (e.g., vasopressin V2 receptor agonist), etc.

Anticholinergic agents include, for example, atropine, scopolamine, homatropine, tropicamide, cyclopentolate, butylscopolamine bromide, propantheline bromide, methylbenactyzium bromide, mepenzolate bromide, pirenzepine, ipratropium bromide, trihexyphenidyl, oxybutynin, propiverine, darifenacin, tolterodine, solifenacin, temiverine, trospium chloride or a salt thereof (e.g., atropine sulfate, scopolamine hydrogen bromide, homatropine hydrogen bromide, cyclopentolate hydrochloride, pirenzepine hydrochloride, trihexyphenidyl hydrochloride, oxybutynin hydrochloride, tolterodine tartrate, solifenacin succinate, etc.), preferably, oxybutynin, propiverine, darifenacin, tolterodine, solifenacin, temiverine, trospium chloride or a salt thereof (e.g., oxybutynin hydrochloride, tolterodine tartrate, solifenacin succinate, etc.). In addition, acetylcholinesterase inhibitors (e.g., distigmine, etc.) and the like can be used.

NK-2 receptor antagonists include, for example, a piperidine derivative such as GR159897, GR149861, SR48968 (saredutant), SR144190, YM35375, YM38336, ZD7944, L-743986, MDL105212A, ZD6021, MDL105172A, SCH205528, SCH62373, R-113281, etc., a perhydroisoindole derivative such as RPR-106145, etc., a quinoline derivative such as SB-414240, etc., a pyrrolopyrimidine derivative such as ZM-253270, etc., a pseudopeptide derivative such as MEN11420 (nepadutant), SCH217048, L-659877, PD-147714 (CAM-2291), MEN10376, S16474, etc., and others such as GR100679, DNK333, GR94800, UK-224671, MEN10376, MEN10627, or a salt thereof, and the like.

The pharmaceutical composition comprising a mixture or combination of the compound of the present invention and the combination drugs may be formulated into

(1) a single formulation as a pharmaceutical composition containing the compound of the present invention and the combination drugs, or

(2) a formulation comprising the compound of the present invention and the combination drugs which are separately formulated. Hereinafter, it is generally briefly referred to as the combination preparation of the present invention.

The combination preparation of the present invention can be formulated by mixing the compound of the present invention and active ingredients of the combination drugs separately or at the same time as itself or with pharmaceutically acceptable carriers in the same manner as in the method of producing the pharmaceutical preparation comprising the compound of the present invention.

A daily dose of the combination preparation of the present invention varies depending on severity of the symptoms, age, sex, weight and sensitivity of the subject to be administered, time and interval of administration, property, formulation and kinds of pharmaceutical preparation, kinds of active ingredients, etc., and is not particularly limited. The dose in terms of the compound of the present invention is not particularly limited if it causes no problems of side effects. In the case of oral administration, a daily dosage is usually in a range of about 0.005 to 100 mg, preferably about 0.05 to 50 mg, and more preferably about 0.2 to 30 mg, per 1 kg body weight of mammals, which may be administered once a day or in two or three divided portions a day.

The dose of the compound or the combination preparation of the present invention may be set within the range such that it causes no problems of side effects. The daily dose as the compound or the combination preparation of the present invention varies depending on severity of symptoms, age, sex, weight and sensitivity of the subject to be administered, time and interval of administration, property, formulation and kinds of pharmaceutical preparation, kinds of active ingredients, etc., and is not particularly limited. In the case of oral administration, a daily dosage in terms of active ingredients is usually in a range of about 0.001 to 2000 mg, preferably about 0.01 to 500 mg, and more preferably about 0.1 to 100 mg, per 1 kg body weight of mammals, which may be administered once a day or in two to four divided portions a day.

In administering the combination preparation of the present invention, the compound of the present invention and the combination drugs may be administered at the same time or, the combination drugs may be administered before administering the compound of the present invention, and vice versa. In case of staggered administration, the time interval varies depending on the active ingredients to be administered, a formulation and an administration route. For example, if the combination drugs are administered first, the compound of the present invention may be administered 1 minute to 3 days, preferably 10 minutes to 1 day, more preferably 15 minutes to 1 hour after administering the combination drugs. If the compound of the present invention is administered first, the combination drugs may be administered 1 minute to 1 day, preferably 10 minutes to 6 hours, more preferably 15 minutes to 1 hour after administering the compound of the present invention.

In a preferred administration method, about 0.001 to 200 mg/kg of the combination drugs formulated as an oral preparation is administered orally and then after about 15 minutes, about 0.005 to 100 mg/kg of the compound of the present invention formulated as an oral preparation is administered orally as a daily dose.

In the combination preparation of the present invention, the content of the compound of the present invention varies depending on the forms of the preparation, but usually in the order of 0.01 to 100 wt %, preferably 0.1 to 50 wt %, and further preferably 0.5 to 20 wt %, relative to the total preparation.

### EXAMPLES

The present invention is further described in detail with reference to Reference Examples, Examples, Preparative Examples and Experimental Example, which are not intended to restrict the invention and may be modified without departing from the scope of the invention.

Elution in the column chromatography in the following Reference Examples and Examples was conducted under observation by TLC (thin layer chromatography), unless otherwise indicated. In the TLC observation, 60F254, TLC plates, produced by Merck & Co., Inc. was used, and the solvent employed as an elution solvent in the column chromatography was used as an eluent. For the detection, a UV detector was used. As silica gel for the column chromatography, Silica Gel 60 (70 to 230 mesh) produced by Merck & Co., Inc. was used. The “room temperature” here means a temperature of generally from about 10° C. to 35° C. For drying extracts, sodium sulfate or magnesium sulfate was used.

The abbreviations used in the following Examples and Reference Examples mean the following:
LC: liquid chromatography
MS: mass spectrum
ESI: electrospray ionization
FAB: fast atom bombardment
M: molecular ion peak
NMR: nuclear magnetic resonance
Hz: hertz
J: coupling constant
m: multiplet
q: quartet
t: triplet
d: doublet
s: singlet
br: broad
dt: double triplet
brs: broad singlet
^{t}Bu: tert-butyl group
Boc: tert-butyloxycarbonyl group
Rt: retention time
N: normal concentration
MPa: mega pascal
wt %: percent by weight
DMF: N,N-dimethylformamide
THF: tetrahydrofuran
DMSO: dimethyl sulfoxide
IPE: diisopropyl ether
TFA: trifluoroacetic acid
HOBt.H₂O: 1-hydroxybenzotriazole hydrate
WSC.HCl: 1-ethyl-3-(dimethylaminopropyl)carbodiimide hydrochloride
NaBH(OAc)₃: sodium triacetoxyborohydride
Pd(PPh₃)₄: tetrakis(triphenylphosphine)palladium
Et₃N: triethylamine

LC-MS in Examples and Reference Examples was measured under the following conditions.

Analysis by LC-MS

Measurement instrument: LC-MS system, Waters Corporation
HPLC part: HP1100, Agilent Technologies, Inc.
MS part: Micromass ZMD
   HPLC Conditions
Column: CAPCELL PAK C18UG120, S-3 μm, 1.5×35 mm (Shiseido Co., Ltd.)
Solvent: Solution A; 0.05% TFA-containing water, Solution B; 0.05% TFA-containing acetonitrile
Gradient cycle: 0.00 minute (Solution A/Solution B=90/10), 2.00 minutes (Solution A/Solution B=5/95), 2.75 minutes (Solution A/Solution B=5/95), 2.76 minutes (Solution A/Solution B=90/10), 3.60 minutes (Solution A/Solution B=90/10)
Injection volume: 2 μL, Flow rate: 0.5 mL/min,
Detection method: UV 220 nm
   MS Conditions
Ionization method: ESI
   Chiral HPLC Conditions (Measurement of Diastereomer Excess and Enantiomer Excess in Reference Example 1)
Column: CHIRALCEL OD-RH 4.6 mmID×150 mm
Solvent: 50 mM potassium dihydrogen phosphate (pH 8.0)/acetonitrile=85/15
Injection volume: 20 μL
Flow rate: 0.3 mL/min
Temperature: 40° C.
Detection method: UV 220 nm

In Examples, the reaction by microwave was carried out in the following apparatus.
Instrument: Biotage, Emrys Optimizer

### Reference Example 1

### N-{2-[(3R,4S)-4-amino-3-phenylpiperidin-1-yl]-2-oxoethyl}acetamide methanesulfonate

N-Acetylglycine (6.44 g) was suspended in acetonitrile (120 mL). 3-Phenylpiperidin-4-one monohydrochloride (10.58 g), Et₃N (5.06 g) and WSC.HCl (11.50 g) were successively added thereto, and the mixture was stirred at 50° C. for 2 hr. After allowing to cool to 25° C., brine/3N hydrochloric acid (1:1) (40 mL) was added to partition the mixture. The aqueous layer was extracted again with acetonitrile (60 mL). The organic layers were combined, and the mixture was washed twice successively with brine/5N aqueous sodium hydroxide solution (1:1) (40 mL) and brine (40 mL). The organic layer was concentrated under reduced pressure, and azeotropically concentrated with ethyl acetate. Ethyl acetate (150 mL) and silica gel (10 g) were added to the residue. The mixture was heated to 70° C. and stirred for 30 min. The mixture was heated, and the silica gel was filtered off and washed twice with ethyl acetate (100 mL). The filtrate was concentrated under reduced pressure and azeotropically concentrated with toluene. Toluene (100 mL) was added to the residue and the residue was dissolved by refluxing. After allowing to cool to 25° C., the precipitated crystals were collected by filtration, and washed twice with toluene (20 mL). Drying under reduced pressure gave N-[2-oxo-2-(4-oxo-3-phenylpiperidin-1-yl)ethyl]acetamide (8.70 g) as white crystals.

The obtained N-[2-oxo-2-(4-oxo-3-phenylpiperidin-1-yl)ethyl]acetamide (10 g) was suspended in toluene (50 mL). (S)-1-Phenylethylamine (6.63 g) and p-toluenesulfonic acid monohydrate (0.35 g) were successively added thereto. While refluxing at 110° C. for 3 hr, water was separated with a Dean-Stark apparatus. The mixture was cooled to 25° C. Raney-nickel catalyst (30 mL), ethanol (50 mL) and Et₃N (3.69 g) were added. A reduction reaction was carried out at 50° C. under a hydrogen pressure of 0.5-1 MPa until the hydrogen absorption ceased. The reaction mixture was filtered with pressurization in a nitrogen stream, and the Raney-nickel catalyst was washed twice with ethanol (10 mL). The filtrate and washings were concentrated under reduced pressure. Water (100 mL) was added to the residue and the mixture was refluxed for 30 min. After cooling to room temperature, seed crystals were added and the mixture was stirred for 2 hr. The precipitated crystals were collected by filtration, and washed twice with water (50 mL). Drying in vacuo at 60° C. for 3 hr gave N-[2-oxo-2-((3R,4S)-3-phenyl-4-{[(1S)-1-phenylethyl]amino}piperidin-1-yl)ethyl]acetamide (11.64 g) as white crystals.

The obtained N-[2-oxo-2-((3R,4S)-3-phenyl-4-{[(1S)-1-phenylethyl]amino}piperidin-1-yl)ethyl]acetamide (10 g) was dissolved in ethanol (200 mL). 10% Palladium carbon (water wet) (5 g) was added and a reduction reaction was carried out at 50° C. under a hydrogen pressure of 0.5-1 MPa until the hydrogen absorption ceased. The reaction mixture was filtered, and palladium carbon was washed twice with ethanol (20 mL). The filtrate and washings were concentrated under reduced pressure to give N-{2-[(3R,4S)-4-amino-3-phenylpiperidin-1-yl]-2-oxoethyl}acetamide (7.00 g).

To the obtained N-{2-[(3R,4S)-4-amino-3-phenylpiperidin-1-yl]-2-oxoethyl}acetamide (7.00 g) was added ethanol (75 mL) and the mixture was dissolved by refluxing for 30 min. After cooling to 65° C., methanesulfonic acid (2.53 g) was added thereto. After cooling to 25° C., ethyl acetate (150 mL) was added. The precipitated crystals were collected by filtration, and washed twice with ethanol/ethyl acetate (1:3) (40 mL). Drying under reduced pressure gave white crystals (9.07 g). Ethanol (75 mL) was added thereto and the mixture was dissolved by refluxing for 30 min. After cooling to 25° C., the mixture was stirred for 3 hr and ethyl acetate (150 mL) was added. The precipitated crystals were collected by filtration, and washed twice with ethanol/ethyl acetate (1:3) (40 mL). Drying under reduced pressure gave the title compound (8.84 g) as white crystals.

¹H-NMR (300 MHz, DMSO-d₆): δ 1.83-1.91 (2H, m), 1.88 (3H, s), 2.33 (3H, s), 3.15 (1H, br), 3.58-4.06 (7H, m), 7.30-7.40 (5H, m), 7.78 (3H, br), 7.96-8.03 (1H, m) MS(FAB): 372(M+H) Elemental analysis: C₁₆H₂₅N₃O₅S.1.5H₂O Found C, 48.12; H, 7.00; N, 10.59; S, 8.27; Calcd. C, 48.23; H, 7.08; N, 10.55; S, 8.05. Diastereomer excess: 99.8% de Enantiomer excess: 99.7% ee

### Reference Example 2

### N-(2-{(3R,4S)-4-[(5-bromo-2-methoxybenzyl)amino]-3-phenylpiperidin-1-yl}-2-oxoethyl)acetamide

To a solution of the compound (6.2 g) obtained in Reference Example 1, 5-bromo-2-methoxybenzaldehyde (3.3 g) and acetic acid (2 mL) in methylene chloride. (90 mL) was added NaBH(OAc)₃ (9.7 g), and the mixture was stirred at room temperature for 16 hr. A saturated aqueous sodium hydrogen carbonate solution was added to the reaction mixture, and the mixture was extracted with ethyl acetate. The organic layer was washed with a saturated aqueous sodium hydrogen carbonate solution and then with brine, dried over anhydrous magnesium sulfate and concentrated. The residue was purified by silica gel column chromatography (solvent gradient; 50→100% ethyl acetate/hexane) and crystallized from acetone/IPE to give the title compound (4.68 g, 60%) as white crystals.

MS(ESI+): 474, 476(M+H)

### Reference Example 3

### 4-methoxy-2′-(methylthio)biphenyl-3-carbaldehyde

A mixture of 5-bromo-2-methoxybenzaldehyde (10 g), [2-(methylthio)phenyl]boronic acid (7.8 g), Pd(PPh₃)₄ (1.6 g) and potassium carbonate (12.8 g) in THF/water (2/1) (150 mL) was heated under reflux for 18 hr under a nitrogen atmosphere. After cooling, the reaction mixture was concentrated, and the residue was partitioned between ethyl acetate and water. The organic layer was washed with a saturated aqueous sodium hydrogen carbonate solution and then with brine, dried over anhydrous magnesium sulfate and concentrated. The residue was purified by silica gel column chromatography (solvent gradient; 30→70% ethyl acetate/hexane) to give the title compound (11.5 g, 96%) as a colorless oil.

¹H-NMR (300 MHz, CDCl₃): δ 2.36 (3H, s), 3.96 (3H, s), 7.04 (1H, d, J=8.5 Hz), 7.19 (1H, d, J=3.6 Hz), 7.25-7.36 (3H, m), 7.64 (1H, dd, J=8.5, 2.4 Hz), 7.89 (1H, d, J=2.4 Hz), 10.5 (1H, s)

### Reference Example 4

### 4-methoxy-2′-(methylsulfonyl)biphenyl-3-carbaldehyde

A solution of the compound (8.5 g) obtained in Reference Example 3 and m-chloroperbenzoic acid (13.1 g) in methylene chloride (100 mL) was stirred at room temperature for 4 hr. Ethanol (1 mL), ethyl acetate and a saturated aqueous sodium hydrogen carbonate solution were added to the reaction mixture, and the mixture was extracted with ethyl acetate. The organic layer was washed with a saturated aqueous potassium carbonate solution, a saturated aqueous sodium hydrogen carbonate solution, and then brine, dried over anhydrous magnesium sulfate and concentrated. The residue was purified by silica gel column chromatography (solvent gradient; 30→70% ethyl acetate/hexane) and crystallized from ethanol/IPE to give the title compound (4.36 g, 46%) as white crystals.

Elemental analysis: C₁₅H₁₄O₄S Found C, 61.89; H, 4.79; Calcd. C, 62.05; H, 4.86.

### Reference Examples 5-7

Using (3-formyl-4-methoxyphenyl)boronic acid and bromobenzene derivative (1-bromo-4-ethynylbenzene for Reference Example 5, 4-bromo-2-fluorobenzonitrile for Reference Example 6, and 4-bromo-3-methylbenzonitrile for Reference Example 7), the compounds of Reference Examples 5-7 were obtained in the same manner as in the method described in Reference Example 3.

### Reference Example 5

### 4′-ethynyl-4-methoxybiphenyl-3-carbaldehyde

Elemental analysis: C₁₆H₁₂O₂.0.5H₂O Found C, 78.39; H, 5.03; Calcd. C, 78.35; H, 5.34.

### Reference Example 6

### 3-fluoro-3′-formyl-4′-methoxybiphenyl-4-carbonitrile

Elemental analysis: C₁₅H₁₀FNO₂ Found C, 70.24; H, 3.95; N, 5.29; Calcd. C, 70.58; H, 3.95; N, 5.49.

### Reference Example 7

### 3′-formyl-4′-methoxy-2-methylbiphenyl-4-carbonitrile

Elemental analysis: C₁₆H₁₃NO₂.0.3H₂O Found C, 74.86; H, 5.14; N, 5.10; Calcd. C, 74.87; H, 5.34; N, 5.46.

### Reference Example 8

### 3′-formyl-4′-(trifluoromethoxy)biphenyl-4-carbonitrile

To a solution of diisopropylamine (3.5 mL) in THF (50 mL) was added a 1.6 mol/L n-butyllithium in hexane solution (15 mL) at 0° C. After cooling the reaction mixture to −78° C., 1-bromo-4-(trifluoromethoxy)benzene (6.0 g) was added, and the mixture was stirred for 2 hr. Then, morpholine-4-carbaldehyde (8.6 g) was added to the reaction mixture, and the mixture was concentrated to dryness under reduced pressure. Water was added to the residue, and the mixture was extracted with ethyl acetate. The organic layer was washed with a saturated aqueous sodium hydrogen carbonate solution and then with brine, dried over anhydrous magnesium sulfate and concentrated. The residue was purified by silica gel column chromatography (solvent gradient; 0→30% ethyl acetate/hexane) to give 5-bromo-2-(trifluoromethoxy)benzaldehyde (5.04 g, 75%) as a colorless oil.

A mixture of the obtained 5-bromo-2-(trifluoromethoxy)benzaldehyde (4.2 g), (4-cyanophenyl)boronic acid (2.3 g), Pd(PPh₃)₄ (541 mg) and potassium carbonate (4.3 g) in THF/water (2/1) (60 mL) was heated under reflux for 12 hr under a nitrogen atmosphere. After cooling, the reaction mixture was concentrated, and to the residue were added ethyl acetate and a saturated aqueous sodium hydrogen carbonate solution for extraction. The organic layer was washed with a saturated aqueous sodium hydrogen carbonate solution and then with brine, dried over anhydrous magnesium sulfate and concentrated. The residue was purified by silica gel column chromatography (solvent gradient; 0→40% ethyl acetate/hexane) and crystallized from acetone/IPE to give the title compound (2.45 g, 54%) as white crystals.

Elemental analysis: C₁₅H₈F₃NO₂ Found C, 61.92; H, 2.91; N, 4.96; Calcd. C, 61.86; H, 2.77; N, 4.81.

### Reference Example 9

### [3-({[(3R,4S)-1-(N-acetylglycyl)-3-phenylpiperidin-4-yl]amino}methyl)-4-methoxyphenyl]boronic acid

To a solution of the compound (2.04 g) obtained in Reference Example 1, (3-formyl-4-methoxyphenyl)boronic acid (0.9 g) and acetic acid (0.4 mL) in methylene chloride (40 mL) was added NaBH(OAc)₃ (3.2 g), and the mixture was stirred at room temperature for 15 hr. The reaction mixture was extracted with water. The aqueous layer was purified with DOWEX 50WX8-100 ion exchange resin to give the title compound (1.39 g, 58%) as a pale-yellow oil.

MS(ESI+): 440(M+H)

The compounds described in Reference Examples 1-9 are as follows (Table 1).

TABLE 1

| Ref. | |
|---|---|
| Ex. | Structural |
| No. | formula |
| 1 | 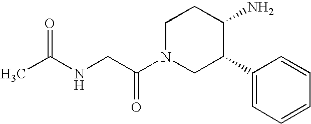 |
| 2 | 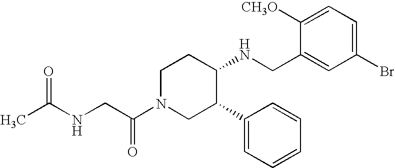 |
| 3 | 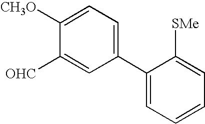 |
| 4 | 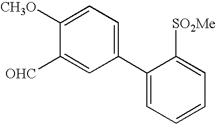 |
| 5 | 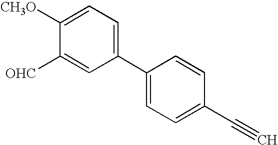 |
| 6 | 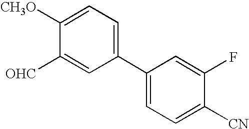 |
| 7 | 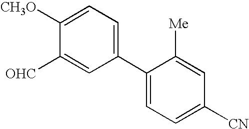 |
| 8 | 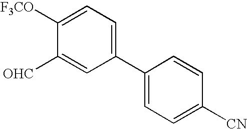 |
| 9 | 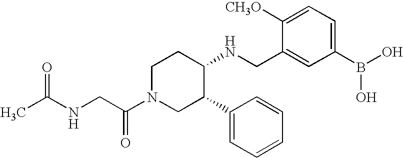 |

### Reference Example 10

### 4′-acetyl-4-methoxybiphenyl-3-carbaldehyde

Using 4′-bromoacetophenone and (3-formyl-4-methoxyphenyl)boronic acid, the title compound was obtained in the same manner as in the method described in Reference Example 3.

Melting point: 136-138° C.

### Reference Example 11

### N-(3′-formyl-4′-methoxybiphenyl-4-yl)acetamide

Using 4-bromoacetanilide and (3-formyl-4-methoxyphenyl)boronic acid, the title compound was obtained in the same manner as in the method described in Reference Example 3.

Elemental analysis: C₁₆H₁₅NO₃.0.5H₂O Found C, 69.11; H, 5.40; N, 4.96; Calcd. C, 69.05; H, 5.79; N, 5.03.

### Reference Example 12

### 4′-tert-butyl-4-methoxybiphenyl-3-carbaldehyde

Using 1-bromo-4-tert-butylbenzene and (3-formyl-4-methoxyphenyl)boronic acid, the title compound was obtained in the same manner as in the method described in Reference Example 3.

Elemental analysis: C₁₈H₂₀O₂.0.2H₂O Found C, 79.24; H, 7.22; Calcd. C, 79.50; H, 7.56.

### Reference Example 13

### 4′-(dimethylamino)-4-methoxybiphenyl-3-carbaldehyde

Using 4-bromo-N,N-dimethylaniline and (3-formyl-4-methoxyphenyl)boronic acid, the title compound was obtained in the same manner as in the method described in Reference Example 3.

Melting point: 113-115° C.

### Reference Example 14

### 4-methoxy-4′-(methylsulfonyl)biphenyl-3-carbaldehyde

Using 4-bromophenyl methyl sulfone and (3-formyl-4-methoxyphenyl)boronic acid, the title compound was obtained in the same manner as in the method described in Reference Example 3.

Elemental analysis: C₁₅H₁₄O₄S Found C, 62.00; H, 4.82; Calcd. C, 62.05; H, 4.86.

### Reference Example 15

### 3′-fluoro-5′-formyl-4′-methoxybiphenyl-4-carbonitrile

(Step 1)

To a solution of 5-bromo-3-fluoro-2-hydroxybenzaldehyde (6.57 g) synthesized by a known method (e.g., Synthesis, Vol. 11, pp. 1878-1880 (1999)) and calcium carbonate (5.80 g) in a mixture of acetone (20 mL) and THF (10 mL) was added methyl iodide (3.74 mL) at room temperature, and the mixture was heated under reflux for 2 hr. Water was added to the reaction mixture, and the mixture was extracted with ethyl acetate. The organic layer was washed with brine, dried over anhydrous sodium sulfate and concentrated. The residue was purified by silica gel column chromatography (solvent gradient; 10→17% ethyl acetate/hexane) to give 5-bromo-3-fluoro-2-methoxybenzaldehyde (3.69 g) as white crystals.

¹H-NMR (300 MHz, CDCl₃): δ 4.10 (3H, d, J=2.7 Hz), 7.48 (1H, dd, J=11.1, 2.4 Hz), 7.73 (1H, dd, J=2.4, 1.8 Hz), 10.33 (1H, s)

(Step 2)

Using the compound obtained in Step 1 and 4-cyanophenylboronic acid, the title compound was obtained in the same manner as in the method described in Reference Example 3.

¹H-NMR (300 MHz, CDCl₃): δ 4.18 (3H, d, J=3 Hz), 7.58 (1H, dd, =13, 2 Hz), 7.64-7.68 (2H, m), 7.73-7.77 (2H, m), 7.86 (1H, dd, J=2, 1 Hz), 10.45 (1H, s)

### Reference Example 16

### methyl 3′-formyl-4′-methoxybiphenyl-4-carboxylate

Using methyl 4-bromobenzoate and (3-formyl-4-methoxyphenyl)boronic acid, the title compound was obtained in the same manner as in the method described in Reference Example 3.

Elemental analysis: C₁₆H₁O₄.0.1H₂O Found C, 70.32; H, 5.18; Calcd. C, 70.63; H, 5.26.

### Reference Example 17

### 4′-chloro-4-methoxy-2′-methylbiphenyl-3-carbaldehyde

Using 2-bromo-5-chlorotoluene and (3-formyl-4-methoxyphenyl)boronic acid, the title compound was obtained in the same manner as in the method described in Reference Example 3.

Elemental analysis: C₁₅H₁₃ClO₂.0.1H₂O Found C, 68.45; H, 4.96; Calcd. C, 68.63; H, 5.07.

### Reference Example 18

### 4′-bromo-4-methoxy-3′-(trifluoromethyl)biphenyl-3-carbaldehyde

Using 2,5-dibromobenzotrifluoride and (3-formyl-4-methoxyphenyl)boronic acid, the title compound was obtained in the same manner as in the method described in Reference Example 3.

Elemental analysis: C₁₅H₁₀BrF₃O₂.0.1H₂O Found C, 51.07; H, 3.15; Calcd. C, 49.91; H, 2.85.

### Reference Example 19

### 2′-fluoro-3′-formyl-4′-methoxybiphenyl-4-carbonitrile

(Step 1)

A solution of 4-chloro-3-fluoroanisole (9.0 g) in THF (20 mL) was added to a 0.51M lithium diisopropylamide/THF solution (120 mL) at −78° C. and the mixture was stirred for 30 min. DMF (6.5 mL) was added and the reaction mixture was stirred at −30° C. Water was added and the mixture was extracted with ethyl acetate. The organic layer was washed with brine, dried over anhydrous sodium sulfate and concentrated to give 3-chloro-2-fluoro-6-methoxybenzaldehyde (7.6 g) as white crystals.

¹H-NMR (300 MHz, CDCl₃): δ 3.94 (3H, s), 6.76 (1H, dd, J=9, 2 Hz), 7.51-7.57 (1H, m), 10.40 (1H, d, J=1 Hz)

(Step 2)

A solution of the compound (1.3 g) obtained in Step 1, 4-cyanophenylboronic acid (1.3 g), palladium(II) acetate (0.046 g), 2-(di-tert-butylphosphino)biphenyl (0.12 g) and potassium carbonate (1.43 g) in a mixture of water (5 mL) and THF (25 mL) was heated under reflux for 2 hr under an argon atmosphere. Water was added to the reaction mixture, and the product was extracted with ethyl acetate. The organic layer was washed with a saturated aqueous sodium hydrogen carbonate solution and brine, dried over anhydrous sodium sulfate and concentrated to give the title compound (0.81 g, 46%) as a white powder.

¹H-NMR (300 MHz, CDCl₃): δ 4.00 (3H, s), 6.91 (1H, dd, J=9, 1.5 Hz), 7.57-7.63 (3H, m), 7.71-7.75 (2H, m), 10.49 (1H, d, J=1.2 Hz)

### Reference Example 20

### 3′-formyl-4′-methoxy-2-(trifluoromethyl)biphenyl-4-carbonitrile

Using 4-chloro-3-(trifluoromethyl)benzonitrile and (3-formyl-4-methoxyphenyl)boronic acid, the title compound was obtained in the same manner as in the method described in Reference Example 3.

Melting point: 116-118° C.

### Reference Example 21

### 3′-formyl-4′-methoxybiphenyl-4-carboxamide

Using 4-bromobenzamide and (3-formyl-4-methoxyphenyl)boronic acid, the title compound was obtained in the same manner as in the method described in Reference Example 3.

Elemental analysis: C₁₅H₁₃NO₃ Found C, 70.46; H, 5.09; N, 5.49; Calcd. C, 70.58; H, 5.13; N, 5.49.

### Reference Example 22

### 2′-fluoro-5′-formyl-4′-methoxybiphenyl-4-carbonitrile

(Step 1)

To a solution of 4-fluoro-2-methoxybenzoic acid (1.70 g) in acetic acid (20 mL) was added bromine (0.56 mL) at room temperature, and the mixture was stirred for 2 hr. Water was added to the reaction mixture, and the precipitate was collected by filtration with water and hexane to give 5-bromo-4-fluoro-2-methoxybenzoic acid (2.38 g, 96%) as a white powder.

¹H-NMR (300 MHz, CDCl₃): δ 4.07 (3H, s), 6.86 (1H, d, J=9.6 Hz), 8.39 (1H, d, J=7.8 Hz)

(Step 2)

To a solution of the compound (2.37 g) obtained in Step 1 and Et₃N (1.99 mL) in THF (25 mL) was added isobutyl chloroformate (1.48 mL) at room temperature, and the mixture was stirred for 30 min. The precipitate was collected by filtration with water and THF. The obtained precipitate was dissolved in a solution of NaBH₄ (1.08 g) in water (12 mL), and the mixture was stirred at room temperature for 1 hr. 1N Hydrochloric acid was added to the reaction mixture, and the product was extracted with ethyl acetate. The organic layer was washed with brine, dried over anhydrous sodium sulfate and concentrated. The obtained residue was purified by silica gel column chromatography (NH Chromatorex) (solvent gradient; 0→25% ethyl acetate/hexane) to give (5-bromo-4-fluoro-2-methoxyphenyl)methanol (2.33 g, 100%) as a colorless oil.

¹H-NMR (300 MHz, CDCl₃): δ 3.84 (3H, s), 4.62 (2H, s), 6.68 (1H, d, J=10.5 Hz), 7.46 (1H, d, J=7.8 Hz)

(Step 3)

To a solution of the compound (2.24 g) obtained in Step 2 in toluene (30 mL) was added manganese dioxide (17.7 g) at room temperature, and the mixture was stirred for 2 days. The insoluble material was filtered off, and the filtrate was concentrated. The obtained residue was purified by silica gel column chromatography (10% ethyl acetate/hexane) to give 5-bromo-4-fluoro-2-methoxybenzaldehyde (1.86 g, 84%) as a white powder.

¹H-NMR (300 MHz, CDCl₃): δ 3.93 (3H, s), 6.79 (1H, d, J=9.9 Hz), 8.03 (1H, d, J=8, 1 Hz), 10.30 (1H, s)

(Step 4)

Using the compound obtained in Step 3 and (4-cyanophenyl)boronic acid, the title compound was obtained in the same manner as in the method described in Reference Example 3.

¹H-NMR (300 MHz, CDCl₃): δ 4.00 (3H, s), 6.84 (1H, d, J=12.3 Hz), 7.62-7.95 (4H, m), 7.99 (1H, d, J=9.0 Hz), 10.42 (1H, s)

### Reference Example 23

### 5′-formyl-4′-methoxy-2′-methylbiphenyl-4-carbonitrile

(Step 1)

To a solution of 4-bromo-3-methylanisole (2.0 g) and dichloromethyl methyl ether (1.1 mL) in methylene chloride (30 mL) was added titanium tetrachloride (1.3 mL) at 0° C., and the mixture was stirred at 0° C. for 2 hr. A saturated aqueous ammonium chloride solution was added to the reaction mixture, and the product was extracted with ethyl acetate. The organic layer was washed with brine, dried over anhydrous sodium sulfate, and concentrated to give 5-bromo-2-methoxy-4-methylbenzaldehyde (0.73 g, 32%) as a white powder.

¹H-NMR (300 MHz, CDCl₃): δ 2.45 (3H, s), 3.91 (3H, s), 6.87 (1H, s), 7.94 (1H, s), 10.33 (1H, s)

(Step 2)

Using the compound obtained in Step 1 and (4-cyanophenyl)boronic acid, the title compound was obtained in the same manner as in the method described in Reference Example 3.

¹H-NMR (300 MHz, CDCl₃): δ 2.33 (3H, s), 3.98 (3H, s), 6.91 (1H, s), 7.38-7.42 (2H, m), 7.68-7.72 (3H, m), 10.44 (1H, s)

### Reference Example 24

### 3′-formyl-4′-hydroxybiphenyl-4-carbonitrile

(Step 1)

To a solution of 5-bromosalicylaldehyde (6.03 g) and 2,6-lutidine (5.2 mL) in methylene chloride (100 mL) was added triisopropylsilyl trifluoromethanesulfonate (9.7 mL) at −78° C., and the mixture was stirred at −30° C. for 3 hr. A saturated aqueous sodium hydrogen carbonate solution was added to the reaction mixture, and the product was extracted with ethyl acetate. The organic layer was washed with brine, dried over anhydrous sodium sulfate and concentrated. The obtained residue was purified by silica gel column chromatography (solvent gradient; 0→2% ethyl acetate/hexane) to give 5-bromo-2-(triisopropylsilyloxy)benzaldehyde (7.21 g, 67%) as a white powder.

¹H-NMR (300 MHz, CDCl₃): δ 1.12 (18H, d, J=7.2 Hz), 1.29-1.56 (3H, m), 6.79 (1H, d, J=7.9 Hz), 7.51 (1H, dd, J=8.7, 2.7 Hz), 7.81 (1H, d, J=2.7 Hz), 10.44 (1H, s)

(Step 2)

A solution of the compound (1.6 g) obtained in Step 1, (4-cyanophenyl)boronic acid (0.73 g), Pd(PPh₃)₄ (0.16 g) and potassium carbonate (0.88 g) in a mixture of THF/water (13/1) (21.5 mL) was heated under reflux for 14 hr under a nitrogen atmosphere. After cooling, the reaction mixture was extracted with ethyl acetate. The organic layer was washed with brine, dried over anhydrous sodium sulfate and concentrated. The residue was purified by silica gel column chromatography (20% ethyl acetate/hexane) to give the title compound (0.63 g, 62%) as a pale-yellow powder.

¹H-NMR (300 MHz, CDCl₃): δ 7.11-7.14 (1H, m), 7.64-7.79 (6H, m), 9.99 (1H, s), 11.10 (1H, s)

### Reference Example 25

### 3′-formyl-4′-methoxy-2′-methylbiphenyl-4-carbonitrile

(Step 1)

To a solution of 4-bromo-3-methylanisole (10.1 g) and dichloromethyl methyl ether (5.4 mL) in methylene chloride (150 mL) was added titanium tetrachloride (6.6 mL) at 0° C., and the mixture was stirred at 0° C. for 2 hr. A saturated aqueous ammonium chloride solution was added to the reaction mixture, and the product was extracted with ethyl acetate. The organic layer was washed with brine, dried over anhydrous sodium sulfate and concentrated. The residue was purified by silica gel column chromatography (solvent gradient; 0→10% ethyl acetate/hexane) to give 3-bromo-6-methoxy-2-methylbenzaldehyde (0.56 g, 5%) as a white powder.

¹H-NMR (300 MHz, CDCl₃): δ 2.64 (3H, s), 3.90 (3H, s), 6.75 (1H, d, J=8.7 Hz), 7.67 (1H, d, J=9.3 Hz), 10.54 (1H, s)

(Step 2)

Using the compound obtained in Step 1 and (4-cyanophenyl)boronic acid, the title compound was obtained in the same manner as in the method described in Reference Example 3.

¹H-NMR (300 MHz, CDCl₃): δ 2.41 (3H, s), 3.95 (3H, s), 6.92 (1H, d, J=11.4 Hz), 7.32-7.38 (3H, m), 7.68-7.72 (2H, m), 10.68 (1H, s)

### Reference Example 26

### 4-(7-formyl-2,3-dihydro-1-benzofuran-5-yl)benzonitrile

(Step 1)

To a solution of 2,3-dihydrobenzofuran (6.0 g) in acetonitrile (150 mL) was added N-bromosuccinimide (9.8 g) at 0° C., and the mixture was stirred at 0° C. for 2 hr. Water was poured into the reaction mixture, and the mixture was extracted with ethyl acetate. The organic layer was washed with brine, dried over anhydrous sodium sulfate and concentrated. The residue was purified by silica gel column chromatography (33% ethyl acetate/hexane) to give 5-bromo-2,3-dihydro-1-benzofuran (3.8 g, 38%) as a white powder.

¹H-NMR (300 MHz, CDCl₃): δ 3.20 (2H, t, J=8.7 Hz), 4.57 (2H, t, J=8.6 Hz), 6.66 (1H, d, J=9.0 Hz), 7.19 (1H, dd, J=8.7, 2.3 Hz), 7.28 (1H, m)

(Step 2)

Using the compound obtained in Step 1 and (4-cyanophenyl)boronic acid, 4-(2,3-dihydro-1-benzofuran-5-yl)benzonitrile was obtained in the same manner as in the method described in Reference Example 3.

¹H-NMR (300 MHz, CDCl₃): δ 3.29 (2H, t, J=8.6 Hz), 4.65 (2H, t, J=8.7 Hz), 6.88 (1H, d, J=8.3 Hz), 7.35 (1H, dd, J=8.3, 2.2 Hz), 7.44 (1H, d, J=1.5 Hz), 7.60-7.70 (4H, m)

(Step 3)

Using the compound obtained in Step 2, the title compound was obtained in the same manner as in the method described in Reference Example 23, Step 1.

¹H-NMR (300 MHz, CDCl₃): δ 3.34 (2H, t, J=8.6 Hz), 4.83 (2H, t, J=8.7 Hz), 7.64-7.67 (3H, m), 7.71-7.74 (2H, m), 7.84 (1H, m), 10.27 (1H, s)

### Reference Example 27

### 3′-formyl-4′-(2,2,2-trifluoroethoxy)biphenyl-4-carbonitrile

(Step 1)

To a solution of 5-bromosalicylaldehyde (0.81 g) in DMF (8 mL) was added sodium hydride (NaH) (60% in oil, 0.19 g) at room temperature, and the mixture was stirred for 30 min. Then, a solution of 2,2,2-trifluoroethyl trifluoromethanesulfonate (1.1 g) in DMF (8 mL) was added, and the mixture was stirred at 65° C. for 2 hr. Ice water was poured into the reaction mixture, and the mixture was extracted with ethyl acetate. The organic layer was washed with brine, dried over anhydrous sodium sulfate and concentrated. The residue was purified by silica gel column chromatography (solvent gradient; 5→30% ethyl acetate/hexane) to give 5-bromo-2-(2,2,2-trifluoroethoxy)benzaldehyde (1.0 g, 88%) as a colorless oil.

¹H-NMR (300 MHz, CDCl₃): δ 4.48 (2H, q, J=7.7 Hz), 6.86-6.89 (1H, m), 7.66-7.70 (1H, m), 8.00 (1H, d, J=2.7 Hz), 10.41 (1H, s)

(Step 2)

Using the compound obtained in Step 1 and (4-cyanophenyl)boronic acid, the title compound was obtained in the same manner as in the method described in Reference Example 3.

¹H-NMR (300 MHz, CDCl₃): δ 4.57 (2H, q, J=7.5 Hz), 7.10 (1H, d, J=8.7 Hz), 7.67-7.85 (5H, m), 8.15 (1H, d, J=2.3 Hz), 10.54 (1H, s)

### Reference Example 28

### 2-fluoro-3′-formyl-4′-(trifluoromethoxy)biphenyl-4-carbonitrile

(Step 1)

A solution of 5-bromo-2-(trifluoromethoxy)benzaldehyde (5.38 g) synthesized by a known method (Bioorganic & Medicinal Chemistry Letters, 9 (1999), 1311-1316), bis(pinacolato)diboron (5.1 g), potassium acetate (5.9 g) and [1,1′-bis(diphenylphosphino)ferrocene]palladium(II) dichloride (PdCl₂(dppf)) (0.65 g) in DMF (35 mL) was stirred at 90° C. for 16 hr under an argon atmosphere. Water was poured into the reaction mixture, and the mixture was extracted with ethyl acetate. The organic layer was washed with brine, dried over anhydrous magnesium sulfate and concentrated. The residue was purified by silica gel column chromatography (solvent gradient; 5→20% ethyl acetate/hexane) to give crude 5-(4,4,5,5-tetramethyl-1,3,2-dioxaborolan-2-yl)-2-(trifluoromethoxy)benzaldehyde (3.2 g, 51%) as a white powder. The obtained crude product was used in the next step without further purification.

(Step 2)

A solution of the compound (1.6 g) obtained in Step 1, 4-chloro-3-fluorobenzonitrile (0.78 g), Pd(PPh₃)₄ (0.17 g) and potassium carbonate (1.4 g) in a mixture of THF (20 mL) and water (10 mL) was stirred under an argon atmosphere at 90° C. for 16 hr. Water was poured into the reaction mixture, and the mixture was extracted with ethyl acetate. The organic layer was washed with brine, dried over anhydrous magnesium sulfate and concentrated. The residue was purified by silica gel column chromatography (solvent gradient; 5→20% ethyl acetate/hexane) to give the title compound (0.42 g, 27%) as a white powder.

Elemental analysis: C₁₅H₇F₄NO₂ Found C, 58.06; H, 2.46; N, 4.70; Calcd. C, 58.26; H, 2.28; N, 4.53.

### Reference Example 29

### 4′-chloro-2′-fluoro-4-(trifluoromethoxy)biphenyl-3-carbaldehyde

Using 5-bromo-2-(trifluoromethoxy)benzaldehyde and 4-chloro-2-fluorophenylboronic acid, the title compound was obtained in the same manner as in the method described in Reference Example 3.

Melting point: 41-42° C.

### Reference Example 30

### 4-(trifluoromethoxy)-4′-(trifluoromethyl)biphenyl-3-carbaldehyde

Using 5-bromo-2-(trifluoromethoxy)benzaldehyde and 4-(trifluoromethyl)phenylboronic acid, the title compound was obtained in the same manner as in the method described in Reference Example 3.

¹H-NMR (300 MHz, CDCl₃): δ 7.48 (1H, d, J=6.0 Hz), 7.68-7.75 (4H, q like), 7.89 (1H, d, J=6.0 Hz), 8.18 (1H, s), 10.43 (1H, s)

### Reference Example 31

### 4′-chloro-4-(trifluoromethoxy)biphenyl-3-carbaldehyde

Using 5-bromo-2-(trifluoromethoxy)benzaldehyde and 4-chlorophenylboronic acid, the title compound was obtained in the same manner as in the method described in Reference Example 3.

¹H-NMR (300 MHz, CDCl₃): δ 7.42-7.53 (5H, m), 7.81-7.84 (1H, m), 8.14 (1H, s), 10.42 (1H, s)

### Reference Example 32

### 4′-fluoro-4-(trifluoromethoxy)biphenyl-3-carbaldehyde

Using 5-bromo-2-(trifluoromethoxy)benzaldehyde and 4-fluorophenylboronic acid, the title compound was obtained in the same manner as in the method described in Reference Example 3.

¹H-NMR (300 MHz, CDCl₃): δ 7.12-7.18 (2H, m), 7.43 (1H, d, J=6.0 Hz), 7.53-7.57 (2H, m), 7.81 (1H, d, J=6.0 Hz), 8.11 (1H, s), 10.41 (1H, S)

### Reference Example 33

### 2′,4′-dichloro-4-(trifluoromethoxy)biphenyl-3-carbaldehyde

Using 5-bromo-2-(trifluoromethoxy)benzaldehyde and 2,4-dichlorophenylboronic acid, the title compound was obtained in the same manner as in the method described in Reference Example 3.

Melting point: 62-63° C.

### Reference Example 34

### 2′,4′-difluoro-4-(trifluoromethoxy)biphenyl-3-carbaldehyde

Using 5-bromo-2-(trifluoromethoxy)benzaldehyde and 2-(4,4,5,5-tetramethyl-1,3,2-dioxaborolan-2-yl)-1,5-difluorobenzene, the title compound was obtained in the same manner as in the method described in Reference Example 3.

Melting point: 39-41° C.

### Reference Example 35

### 2-chloro-3′-formyl-4′-(trifluoromethoxy)biphenyl-4-carbonitrile

Using the compound obtained in Reference Example 28, Step 1 and 3,4-dichlorobenzonitrile, the title compound was obtained in the same manner as in the method described in Reference Example 3.

Melting point: 134° C.

### Reference Example 36

### 2′-chloro-4′-fluoro-4-(trifluoromethoxy)biphenyl-3-carbaldehyde

Using 5-bromo-2-(trifluoromethoxy)benzaldehyde and 2-chloro-4-fluorophenylboronic acid, the title compound was obtained in the same manner as in the method described-in Reference Example 3.

Melting point: 64° C.

### Reference Example 37

### 4′-(difluoromethoxy)-3′-formylbiphenyl-4-carbonitrile

(Step 1)

Using 5-bromosalicylaldehyde and 4-cyanophenylboronic acid, crude 3′-formyl-4′-hydroxybiphenyl-4-carbonitrile was obtained in the same manner as in the method described in Reference Example 3. The obtained crude product was used in the next step without further purification.

(Step 2)

A solution of the compound (1.0 g) obtained in Step 1 in methylene chloride (40 mL) was added to tetrabutylammonium bromide (1.2 g) in a 50% aqueous potassium hydroxide solution (50 mL) at 5° C. Chloro(difluoro)methane gas (halocarbon 22) was blown into this mixture for 5 min, and chloro(difluoro)methane gas was further blown into the mixture while stirring for 30 min. Water was poured into the reaction mixture, and the mixture was extracted with ethyl acetate. The organic layer was washed with brine, dried over anhydrous magnesium sulfate and concentrated. The residue was purified by silica gel column chromatography (solvent gradient; 10→25% ethyl acetate/hexane) to give the title compound (0.26 g, 21%) as a pale-yellow powder.

¹H-NMR (300 MHz, CDCl₃): δ 6.74 (1H, t, J=72.2 Hz), 7.39 (1H, d, J=8.5 Hz), 7.63-7.80 (4H, m), 7.85 (1H, dd, J=2.5, 8.7 Hz), 8.17 (1H, d, J=2.5 Hz), 10.46 (1H, brs)

### Reference Example 38

### 3′-acetyl-4′-methoxybiphenyl-4-carbonitrile

(Step 1)

To a solution of 3′-formyl-4′-methoxybiphenyl-4-carbonitrile (0.47 g) synthesized by a known method (WO02/26710) in THF (10 mL) was added a 3M methylmagnesium bromide/diethyl ether solution (1 mL) at −78° C., and the mixture was stirred at room temperature for 2 hr. Water was poured into the reaction mixture, and the mixture was extracted with ethyl acetate. The organic layer was washed with brine, dried over anhydrous magnesium sulfate and concentrated. The residue was purified by silica gel column chromatography (solvent gradient; 10→33% ethyl acetate/hexane) to give 3′-(1-hydroxyethyl)-4′-methoxybiphenyl-4-carbonitrile (0.46 g, 91%) as a white amorphous solid.

Elemental analysis: C₁₆H₁₅NO₂ Found C, 75.51; H, 5.91; N, 5.39; Calcd. C, 75.87; H, 5.97; N, 5.53.

(Step 2)

To a solution of the compound (2.0 g) obtained in Step 1 in methylene chloride (70 mL) was added pyridinium dichromate (PDC) (2.73 g), and the mixture was stirred at room temperature for 14 hr. The reaction mixture was treated with silica gel, and the reaction solution was concentrated. The residue was purified by silica gel column chromatography (solvent gradient; 10→33% ethyl acetate/hexane) to give the title compound (1.53 g, 92%) as a white powder.

Melting point: 102° C.

### Reference Example 39

### 3′-formyl-2-methyl-4′-(trifluoromethoxy)biphenyl-4-carbonitrile

Using the compound obtained in Reference Example 28, Step 1 and 4-bromo-3-methylbenzonitrile, the title compound was obtained in the same manner as in the method described in Reference Example 3.

Elemental analysis: C₁₆H₁₀F₃NO₂ Found C, 63.19; H, 3.60; N, 4.19; Calcd. C, 62.96; H, 3.30; N, 4.59.

The compounds described in Reference Examples 10-39 are as follows (Tables 2-3).

TABLE 2

| Ref. | |
|---|---|
| Ex. | Structural |
| No. | formula |
| 10 | 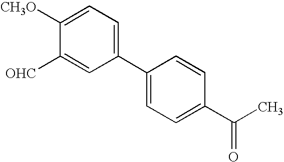 |
| 11 | 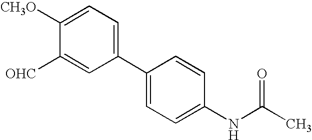 |
| 12 | 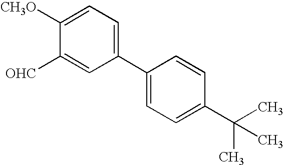 |
| 13 | 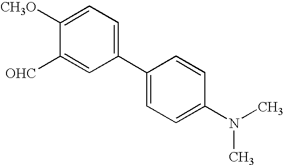 |
| 14 | 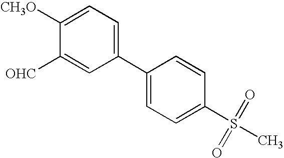 |
| 15 | 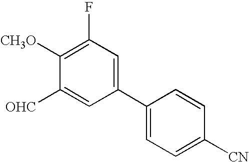 |
| 16 | 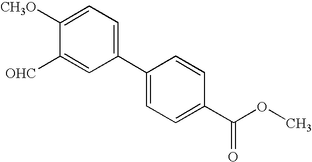 |
| 17 | 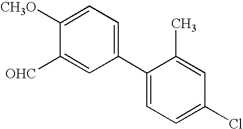 |
| 18 | 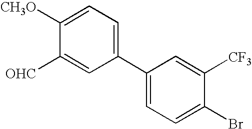 |
| 19 | 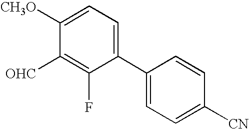 |
| 20 | 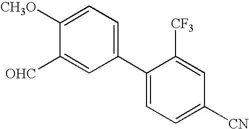 |
| 21 | 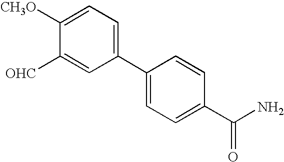 |
| 22 | 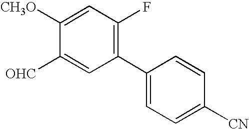 |
| 23 | 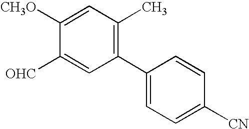 |
| 24 | 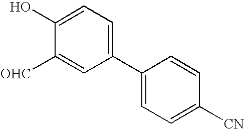 |

TABLE 3

| Ref. | |
|---|---|
| Ex. | Structural |
| No. | formula |
| 25 | 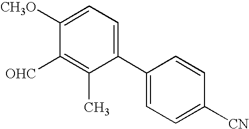 |
| 26 | 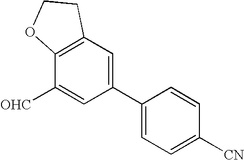 |
| 27 | 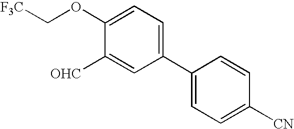 |
| 28 | 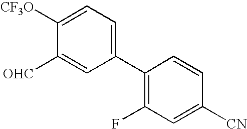 |
| 29 | 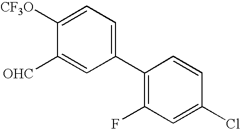 |
| 30 | 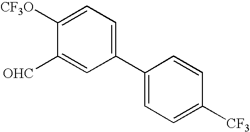 |
| 31 | 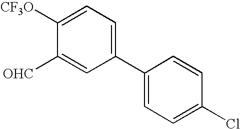 |
| 32 | 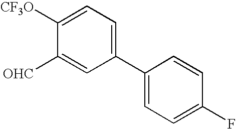 |
| 33 | 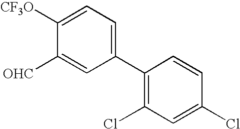 |
| 34 | 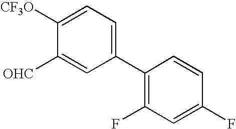 |
| 35 | 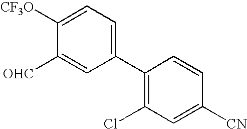 |
| 36 | 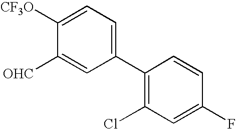 |
| 37 | 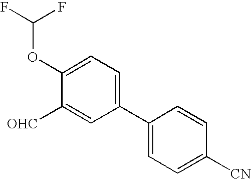 |
| 38 | 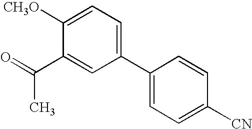 |
| 39 | 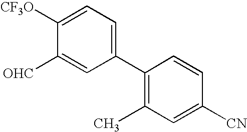 |

### Example 1

### tert-butyl (3R,4S)-4-({[4-methoxy-2′-(methylsulfonyl)biphenyl-3-yl]methyl}amino)-3-phenylpiperidine-1-carboxylate

To a solution of tert-butyl (3R,4S)-4-amino-3-phenylpiperidine-1-carboxylate (912 mg) synthesized by a known method (WO03/101964), the compound (871 mg) obtained in Reference Example 4 and acetic acid (0.3 mL) in methylene chloride (15 mL) was added NaBH(OAc)₃ (2.1 g), and the mixture was stirred at room temperature for 16 hr. A saturated aqueous sodium hydrogen carbonate solution was added to the reaction mixture, and the mixture was extracted with ethyl acetate. The organic layer was washed with a saturated aqueous sodium hydrogen carbonate solution and then with brine, dried over anhydrous magnesium sulfate and concentrated. The residue was purified by silica gel column chromatography (solvent gradient; 0→50% ethyl acetate/hexane) to give the title compound (1.41 g, 85%) as a colorless amorphous solid.

MS(ESI+): 551(M+H)

### Examples 2-3

Using tert-butyl (3R,4S)-4-amino-3-phenylpiperidine-1-carboxylate and the respectively corresponding benzaldehyde derivatives (3′-formyl-4′-methoxybiphenyl-4-carbonitrile synthesized by a known method (WO02/26710) for Example 2 and 2-fluoro-3′-formyl-4′-methoxybiphenyl-4-carbonitrile synthesized by a known method (WO02/26710) for Example 3), the compounds of Examples 2-3 were obtained in the same manner as in the method described in Example 1.

### Example 2

### tert-butyl (3R,4S)-4-{[(4′-cyano-4-methoxybiphenyl-3-yl)methyl]amino}-3-phenylpiperidine-1-carboxylate

MS(ESI+): 498(M+H)

### Example 3

### tert-butyl (3R,4S)-4-{[(4′-cyano-2′-fluoro-4-methoxybiphenyl-3-yl)methyl]amino}-3-phenylpiperidine-1-carboxylate

MS(ESI+): 516(M+H)

### Example 4

### (3R,4S)-N-{[4-methoxy-2′-(methylsulfonyl)biphenyl-3-yl]methyl}-3-phenylpiperidin-4-amine dihydrochloride

To a solution of the compound (1.2 g) obtained in Example 1 in ethanol (10 mL) was added a 4N hydrogen chloride/ethyl acetate (10 mL) solution, and the mixture was stirred under heating at 60° C. for 4 hr. The reaction mixture was concentrated to dryness and recrystallized from ethanol/ethyl acetate/IPE to give the title compound (890 mg, 84%) as white crystals.

MS(ESI+): 451(M−2HCl+H)

### Examples 5-6

Using the compounds obtained in Examples 2-3, the compounds of Examples 5-6 were obtained in the same manner as in the method described in Example 4.

### Example 5

### 4′-methoxy-3′-({[(3R,4S)-3-phenylpiperidin-4-yl]amino}methyl)biphenyl-4-carbonitrile dihydrochloride

MS(ESI+): 398(M−2HCl+H)

### Example 6

### 2-fluoro-4′-methoxy-3′-({[(3R,4S)-3-phenylpiperidin-4-yl]amino}methyl)biphenyl-4-carbonitrile dihydrochloride

MS(ESI+): 416(M−2HCl+H)

### Example 7

### N-[2-((3R,4S)-4-{[(4-methoxybiphenyl-3-yl)methyl]amino}-3-phenylpiperidin-1-yl)-2-oxoethyl]acetamide

To a solution of the compound (408 mg) obtained in Reference Example 1, 4-methoxybiphenyl-3-carbaldehyde (212 mg) synthesized by a known method (WO2005/005415) and acetic acid (0.1 mL) in methylene chloride (10 mL) was added NaBH(OAc)₃ (700 mg), and the mixture was stirred at room temperature for 10 hr. Water was added to the reaction mixture, and the mixture was extracted with ethyl acetate. The organic layer was washed with a saturated aqueous sodium hydrogen carbonate solution and then with brine, dried over anhydrous magnesium sulfate and concentrated. The residue was purified by silica gel column chromatography (solvent gradient; 50→100% ethyl acetate/hexane) and crystallized from acetone/diisopropyl ether to give the title compound (384 mg, 74%) as white crystals.

MS(ESI+): 472(M+H)

### Examples 8-11

Using the compound obtained in Reference Example 1 and the respectively corresponding benzaldehyde derivatives (the compound obtained in Reference Example 3 for Example 8, the compound obtained in Reference Example 4 for Example 9, 3′-formyl-4′-methoxybiphenyl-4-carbonitrile synthesized by a known method (WO02/26710) for Example 10, and 2-fluoro-3′-formyl-4′-methoxybiphenyl-4-carbonitrile synthesized by a known method (WO02/26710) for Example 11), the compounds of Examples 8-11 were obtained in the same manner as in the method described in Example 7 (the compounds of Examples 8 and 9 were respectively treated with 1 equivalent of hydrogen chloride/ethyl acetate and isolated as hydrochlorides).

### Example 8

### N-{2-[(3R,4S)-4-({[4-methoxy-2′-(methylthio)biphenyl-3-yl]methyl}amino)-3-phenylpiperidin-1-yl]-2-oxoethyl}acetamide monohydrochloride

MS(ESI+): 518(M−HCl+H)

### Example 9

### N-{2-[(3R,4S)-4-({[4-methoxy-2′-(methylsulfonyl)biphenyl-3-yl]methyl}amino)-3-phenylpiperidin-1-yl]-2-oxoethyl}acetamide monohydrochloride

MS(ESI+): 550(M−HCl+H)

### Example 10

### N-[2-((3R,4S)-4-{[(4′-cyano-4-methoxybiphenyl-3-yl)methyl]amino}-3-phenylpiperidin-1-yl)-2-oxoethyl]acetamide

MS(ESI+): 497(M+H)

### Example 11

### N-[2-((3R,4S)-4-{[(4′-cyano-2′-fluoro-4-methoxybiphenyl-3-yl)methyl]amino}-3-phenylpiperidin-1-yl)-2-oxoethyl]acetamide

MS(ESI+): 515(M+H)

### Example 12

### N-[2-((3R,4S)-4-{[(4,4′-dimethoxybiphenyl-3-yl)methyl]amino}-3-phenylpiperidin-1-yl)-2-oxoethyl]acetamide

A mixture of the compound (475 mg) obtained in Reference Example 2, (4-methoxyphenyl)boronic acid (152 mg), Pd(PPh₃)₄ (35 mg) and potassium carbonate (276 mg) in THF/water (2/1) (18 mL) was microwaved (250 W) at 150° C. for 15 min. After cooling, the reaction mixture was concentrated, and to the residue were added ethyl acetate and water for extraction. The organic layer was washed with a saturated aqueous sodium hydrogen carbonate solution and then with brine, dried over anhydrous magnesium sulfate and concentrated. The residue was purified by silica gel column chromatography (solvent gradient; 50→100% ethyl acetate/hexane) and crystallized from acetone/diisopropyl ether to give the title compound (273 mg, 54%) as white crystals.

MS(ESI+): 502(M+H)

### Examples 13-18

Using the compound obtained in Reference Example 2 and the respectively corresponding commercially available phenylboronic acid derivatives, the compounds of Examples 13-18 were obtained in the same manner as in the method described in Example 12.

### Example 13

### N-{2-[(3R,4S)-4-({[4-methoxy-4′-(trifluoromethoxy)biphenyl-3-yl]methyl}amino)-3-phenylpiperidin-1-yl]-2-oxoethyl}acetamide

MS(ESI+): 556(M+H)

### Example 14

### N-[2-((3R,4S)-4-{[(4-methoxy-4′-nitrobiphenyl-3-yl)methyl]amino}-3-phenylpiperidin-1-yl)-2-oxoethyl]acetamide

MS(ESI+): 517(M+H)

### Example 15

### N-[2-((3R,4S)-4-{[(4′-chloro-4-methoxybiphenyl-3-yl)methyl]amino}-3-phenylpiperidin-1-yl)-2-oxoethyl]acetamide

MS(ESI+): 506(M+H)

### Example 16

### N-[2-((3R,4S)-4-{[(4′-fluoro-4-methoxybiphenyl-3-yl)methyl]amino}-3-phenylpiperidin-1-yl)-2-oxoethyl]acetamide

MS(ESI+): 490(M+H)

### Example 17

### N-[2-((3R,4S)-4-{[(4-methoxy-4′-methylbiphenyl-3-yl)methyl]amino}-3-phenylpiperidin-1-yl)-2-oxoethyl]acetamide

MS(ESI+): 486(M+H)

### Example 18

### N-{2-[(3R,4S)-4-({[4-methoxy-4′-(trifluoromethyl)biphenyl-3-yl]methyl}amino)-3-phenylpiperidin-1-yl]-2-oxoethyl}acetamide

MS(ESI+): 540(M+H)

### Example 19

### N-[2-((3R,4S)-4-{[(2′-cyano-4-methoxybiphenyl-3-yl)methyl]amino}-3-phenylpiperidin-1-yl)-2-oxoethyl]acetamide

To a solution of the compound obtained in Reference Example 1 (409 mg), 3′-formyl-4′-methoxybiphenyl-2-carbonitrile (237 mg) synthesized by a known method (WO02/26710) and acetic acid (0.2 mL) in methylene chloride (7 mL) was added NaBH(OAc)₃ (636 mg), and the mixture was stirred at room temperature for 12 hr. A saturated aqueous sodium hydrogen carbonate solution was added to the reaction mixture, and the mixture was extracted with ethyl acetate. The organic layer was washed with a saturated aqueous sodium hydrogen carbonate solution and then with brine, dried over anhydrous magnesium sulfate and concentrated. The residue was purified by silica gel column chromatography (solvent gradient; 50→100% ethyl acetate/hexane) and crystallized from acetone/IPE to give the title compound (450 mg, 90%) as white crystals.

MS(ESI+): 497(M+H)

### Examples 20-25

Using the compound obtained in Reference Example 1 and the respectively corresponding benzaldehyde derivatives (3′-formyl-4′-methoxybiphenyl-3-carbonitrile synthesized by a known method (WO02/26710) for Example 20, the compound obtained in Reference-Example 6 for Example 21, 4′-bromo-4-methoxybiphenyl-3-carbaldehyde synthesized by a known method (WO02/26710) for Example 22, the compound obtained in Reference Example 5 for Example 23, the compound obtained in Reference Example 8 for Example 24, and the compound obtained in Reference Example 7 for Example 25), the compounds of Examples 20-25 were obtained in the same manner as in the method described in Example 19.

### Example 20

### N-[2-((3R,4S)-4-{[(3′-cyano-4-methoxybiphenyl-3-yl)methyl]amino}-3-phenylpiperidin-1-yl)-2-oxoethyl]acetamide

MS(ESI+): 497(M+H)

### Example 21

### N-[2-((3R,4S)-4-{[(4′-cyano-3′-fluoro-4-methoxybiphenyl-3-yl)methyl]amino}-3-phenylpiperidin-1-yl)-2-oxoethyl]acetamide

MS(ESI+): 515(M+H)

### Example 22

### N-[2-((3R,4S)-4-{[(4′-bromo-4-methoxybiphenyl-3-yl)methyl]amino}-3-phenylpiperidin-1-yl)-2-oxoethyl]acetamide

MS(ESI+): 550, 552(M+H)

### Example 23

N-[2-((3R,4S)-4-{[(4′-ethynyl-4-methoxybiphenyl-3-yl)methyl]amino}-3-phenylpiperidin-1-yl)-2-oxoethyl]acetamide

MS(ESI+): 496(M+H)

### Example 24

### N-{2-[(3R,4S)-4-({[4′-cyano-4-(trifluoromethoxy)biphenyl-3-yl]methyl}amino)-3-phenylpiperidin-1-yl]-2-oxoethyl}acetamide monohydrochloride

MS(ESI+): 551(M−HCl+H)

### Example 25

### N-[2-((3R,4S)-4-{[(4′-cyano-4-methoxy-2′-methylbiphenyl-3-yl)methyl]amino}-3-phenylpiperidin-1-yl)-2-oxoethyl]acetamide monohydrochloride

MS(ESI+): 511(M−HCl+H)

### Example 26

### N-[2-((3R,4S)-4-{[(4-methoxy-3′,5′-dimethylbiphenyl-3-yl)methyl]amino}-3-phenylpiperidin-1-yl)-2-oxoethyl]acetamide monohydrochloride

A mixture of the compound (440 mg) obtained in Reference Example 9, 1-bromo-3,5-dimethylbenzene (186 mg), Pd(PPh₃)₄ (35 mg) and potassium carbonate (276 mg) in THF/water (2/1) (18 mL) was heated under reflux for 12 hr under a nitrogen atmosphere. After cooling, the reaction mixture was concentrated, and to the residue were added ethyl acetate and a saturated aqueous sodium hydrogen carbonate solution for extraction. The organic layer was washed with a saturated aqueous sodium hydrogen carbonate solution and then with brine, dried over anhydrous magnesium sulfate and concentrated. The residue was purified by silica gel column chromatography (solvent gradient; 50→100% ethyl acetate/hexane), and 1 equivalent of 4N hydrogen chloride/ethyl acetate was added. The precipitate was collected by filtration to give the title compound (320 mg, 60%) as white crystals.

MS(ESI+): 500(M−HCl+H)

### Example 27

### N-{2-[(3R,4S)-4-({[4-methoxy-3′,5′-bis(trifluoromethyl)biphenyl-3-yl]methyl}amino)-3-phenylpiperidin-1-yl]-2-oxoethyl}acetamide

Using the compound obtained in Reference Example 9 and 1-bromo-3,5-bis(trifluoromethyl)benzene, the compound of Example 27 was obtained in the same manner as in the method described in Example 26 (the treatment with 1 equivalent of hydrogen chloride/ethyl acetate was not performed, and the compound was obtained in a free form).

MS(ESI+): 608(M+H)

### Example 28

### tert-butyl [2-((3R,4S)-4-{[(4′-cyano-4-methoxybiphenyl-3-yl)methyl]amino}-3-phenylpiperidin-1-yl)-2-oxoethyl]carbamate

To a solution of the compound (154 mg) obtained in Example 5, Et₃N (148 μL) and N-Boc-glycine (38 mg) in THF (5 mL) were added WSC.HCL (77 mg) and HOBt.H₂O (61 mg), and the mixture was stirred at room temperature for 16 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (solvent gradient; 50→100% ethyl acetate/hexane) to give the title compound (189 mg, 62%) as white crystals.

MS(ESI+): 455(M−Boc+2H)

### Example 29

### 3′-({[(3R,4S)-1-glycyl-3-phenylpiperidin-4-yl]amino}methyl)-4′-methoxybiphenyl-4-carbonitrile dihydrochloride

To a solution of the compound (0.18 g) obtained in Example 28 in ethyl acetate (1 mL) was added 4N hydrogen chloride/ethyl acetate (3 mL), and the mixture was stirred at room-temperature for 18 hr. The reaction mixture was concentrated to dryness to give the title compound (0.18 g, 100%) as a colorless amorphous solid.

MS(ESI+): 455(M−2HCl+H)

### Example 30

### N-[2-((3R,4S)-4-{[(4′-cyano-4-methoxybiphenyl-3-yl)methyl]amino}-3-phenylpiperidin-1-yl)-2-oxoethyl]methanesulfonamide

To a solution of the compound obtained in Example 29 and Et₃N (266 μL) in THF (5 mL) was added methanesulfonyl chloride (45 μL), and the mixture was stirred at room temperature for 14 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (solvent gradient; 50→100% ethyl acetate/hexane) to give the title compound (67 mg, 37%) as white crystals.

MS(ESI+): 533(M+H)

### Example 31

### (3R,4S)-1-(methoxyacetyl)-N-{[4-methoxy-2′-(methylsulfonyl)biphenyl-3-yl]methyl}-3-phenylpiperidin-4-amine monohydrochloride

To a solution of the compound (400 mg) obtained in Example 4, Et₃N (154 mg) and methoxyacetic acid (103 mg) in DMF (5 mL) were added WSC.HCl (218 mg) and HOBt.H₂O (175 mg), and the mixture was stirred at room temperature for 8 hr. Water was poured into the reaction mixture, and the product was extracted with ethyl acetate. The organic layer was washed with a saturated aqueous sodium hydrogen carbonate solution and brine and dried over magnesium sulfate, and the solvent was evaporated under reduced pressure. The obtained residue was purified by silica gel column chromatography (solvent gradient; 50→100% ethyl acetate/hexane) and 1 equivalent of 4N hydrogen chloride/ethyl acetate was added. The precipitate was collected by filtration to give the title compound (240 mg, 60%) as a colorless amorphous solid.

MS(ESI+): 523(M−HCl+H)

### Examples 32-33

Using methoxyacetic acid and the respectively corresponding piperidine derivatives (the compound obtained in Example 5 for Example 32, and the compound obtained in Example 6 for Example 33), the compounds of Examples 32-33 were obtained in the same manner as in the method described in Example 31 (these compounds were not treated with 1 equivalent of hydrogen chloride/ethyl acetate, and obtained in free forms).

### Example 32

### 4′-methoxy-3′-({[(3R,4S)-1-(methoxyacetyl)-3-phenylpiperidin-4-yl]amino}methyl)biphenyl-4-carbonitrile

MS(ESI+): 470(M+H)

### Example 33

### 2-fluoro-4′-methoxy-3′-({[(3R,4S)-1-(methoxyacetyl)-3-phenylpiperidin-4-yl]amino}methyl)biphenyl-4-carbonitrile

MS(ESI+): 488(M+H)

### Example 34

### (3R,4S)-1-[(1-acetylpiperidin-4-yl)carbonyl]-N-{[4-methoxy-2′-(methylsulfonyl)biphenyl-3-yl]methyl}-3-phenylpiperidin-4-amine monohydrochloride

To a solution of the compound (400 mg) obtained in Example 4, Et₃N (154 mg) and 1-acetylpiperidine-4-carboxylic acid (195 mg) in DMF (5 mL) were added WSC.HCL (218 mg) and HOBt.H₂O (175 mg), and the mixture was stirred at room temperature for 8 hr. Water was poured into the reaction mixture, and the product was extracted with ethyl acetate. The organic layer was washed with a saturated aqueous sodium hydrogen carbonate solution and brine and dried over magnesium sulfate, and the solvent was evaporated under reduced pressure. The obtained residue was purified by silica gel column chromatography (solvent gradient; 50→100% ethyl acetate/hexane) and 1 equivalent of 4N hydrogen chloride/ethyl acetate was added. The precipitate was collected by filtration to give the title compound (350 mg, 76%) as white crystals.

MS(ESI+): 604(M−HCl+H)

### Examples 35-36

Using 1-acetylpiperidine-4-carboxylic acid and the respectively corresponding piperidine derivatives (the compound obtained in Example 5 for Example 35, and the compound obtained in Example 6 for Example 36), the compounds of Examples 35-36 were obtained in the same manner as in the method described in Example 34 (these compounds were not treated with 1 equivalent of hydrogen chloride/ethyl acetate, and obtained in free forms).

### Example 35

### 3′-[({(3R,4S)-1-[(1-acetylpiperidin-4-yl)carbonyl]-3-phenylpiperidin-4-yl}amino)methyl]-4′-methoxybiphenyl-4-carbonitrile

MS(ESI+): 551(M+H)

### Example 36

### 3′-[({(3R,4S)-1-[(1-acetylpiperidin-4-yl)carbonyl]-3-phenylpiperidin-4-yl}amino)methyl]-2-fluoro-4′-methoxybiphenyl-4-carbonitrile

MS(ESI+): 569(M+H)

### Example 37

### tert-butyl 4-[((3R,4S)-4-{[(4′-cyano-4-methoxybiphenyl-3-yl)methyl]amino}-3-phenylpiperidin-1-yl)carbonyl]piperidine-1-carboxylate

To a solution of the compound (257 mg) obtained in Example 5, Et₃N (248 μL) and 1-(tert-butoxycarbonyl)piperidine-4-carboxylic acid (163 mg) in THF (5 mL) were added WSC.HCL (173 mg) and HOBt.H₂O (139 mg), and the mixture was stirred at room temperature for 16 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (solvent gradient; 50→100% ethyl acetate/hexane) to give the title compound (290 mg, 87%) as a white amorphous solid.

MS(ESI+): 509(M−Boc+2H)

### Example 38

### 4′-methoxy-3′-({[(3R,4S)-3-phenyl-1-(piperidin-4-ylcarbonyl)piperidin-4-yl]amino}methyl)biphenyl-4-carbonitrile dihydrochloride

Using the compound obtained in Example 37, the title compound was obtained in the same manner as in the method described in Example 29.

MS(ESI+): 509(M−2HCl+H)

### Example 39

### 4′-methoxy-3′-{[((3R,4S)-1-{[1-(methylsulfonyl)piperidin-4-yl]carbonyl}-3-phenylpiperidin-4-yl)amino]methyl}biphenyl-4-carbonitrile

Using the compound obtained in Example 38, the title compound was obtained in the same manner as in the method described in Example 30.

MS(ESI+): 587(M+H)

### Example 40

### N-(2-{4-[((3R,4S)-4-{[(4′-cyano-4-methoxybiphenyl-3-yl)methyl]amino}-3-phenylpiperidin-1-yl)carbonyl]piperidin-1-yl}-2-oxoethyl)acetamide monohydrochloride

Using the compound obtained in Example 38 and N-acetylglycine, the title compound was obtained in the same manner as in the method described in Example 31.

MS(ESI+): 608(M−HCl+H)

### Examples 41-44

Using the compound obtained in Example 5 and the respectively corresponding carboxylic acid derivatives (commercially available carboxylic acid derivatives for Examples 41 and 43, 2,6-dioxopiperidine-4-carboxylic acid synthesized by a known method (e.g., Journal of Organic Chemistry (1973), 38(14), pp. 2489-96) for Example 42, (5-oxo-4,5-dihydro-1H-1,2,4-triazol-3-yl)acetic acid synthesized by a known method (e.g., Australian Journal of Chemistry (1979), 32(1), pp. 161-5) for Example 44), the compounds of Examples 41-44 were obtained in the same manner as in the method described in Example 31 (these compounds were not treated with 1 equivalent of hydrogen chloride/ethyl acetate, and obtained in free forms).

### Example 41

### (3R)-3-(acetylamino)-4-((3R,4S)-4-{[(4′-cyano-4-methoxybiphenyl-3-yl)methyl]amino}-3-phenylpiperidin-1-yl)-4-oxobutanamide

MS(ESI+): 554(M+H)

### Example 42

### 3′-[({(3R,4S)-1-[(2,6-dioxopiperidin-4-yl)carbonyl]-3-phenylpiperidin-4-yl}amino)methyl]-4′-methoxybiphenyl-4-carbonitrile

MS(ESI+): 537(M+H)

### Example 43

### 4′-methoxy-3′-({[(3R,4S)-3-phenyl-1-(1H-tetrazol-1-ylacetyl)piperidin-4-yl]amino}methyl)biphenyl-4-carbonitrile

MS(ESI+): 508(M+H)

### Example 44

### 4′-methoxy-3′-[({(3R,4S)-1-[(5-oxo-4,5-dihydro-1H-1,2,4-triazol-3-yl)acetyl]-3-phenylpiperidin-4-yl}amino)methyl]biphenyl-4-carbonitrile

MS(ESI+): 523(M+H)

### Example 45

### methyl (3R,4S)-4-{[(4′-cyano-4-methoxybiphenyl-3-yl)methyl]amino}-3-phenylpiperidine-1-carboxylate

To a solution of the compound (137 mg) obtained in Example 5 and Et₃N (206 μL) in THF (5 mL) was added methyl chloroformate (34 μL), and the mixture was stirred at room temperature for 16 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (solvent gradient; 50→100% ethyl acetate/hexane) to give the title compound (59 mg, 45%) as white crystals.

MS(ESI+): 456(M+H)

### Example 46

### (3R,4S)-4-{[(4′-cyano-4-methoxybiphenyl-3-yl)methyl]amino}-N-methyl-3-phenylpiperidine-1-carboxamide

To a solution of the compound (173 mg) obtained in Example 5 in THF (5 mL) was added methyl isocyanate (38 μL), and the mixture was stirred at room temperature for 16 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (solvent gradient; 50→70% ethyl acetate/hexane) to give the title compound (40 mg, 24%) as white crystals.

MS(ESI+): 455(M+H)

The compounds described in Examples 1-46 are as follows (Tables 4-7).

TABLE 4

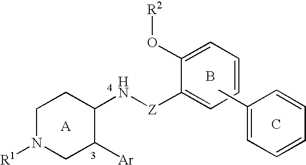

| Ex. No. | R¹ | 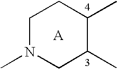 | Ar | Z | 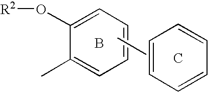 | Addi- tives | MS (ESI) |
|---|---|---|---|---|---|---|---|
| 1 | 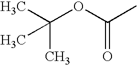 | 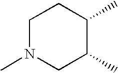 (3R, 4S) | 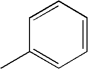 | CH₂ | 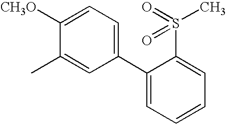 | | 551 (M + H)⁺ |
| 2 | 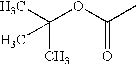 | 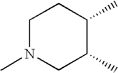 (3R, 4S) | 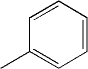 | CH₂ | 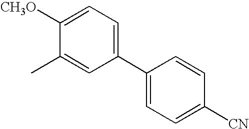 | | 498 (M + H)⁺ |
| 3 | 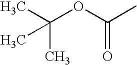 | 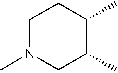 (3R, 4S) | 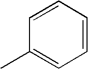 | CH₂ | 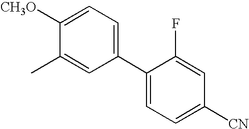 | | 516 (M + H)⁺ |
| 4 | H | 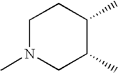 (3R, 4S) | 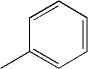 | CH₂ | 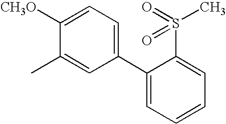 | 2HCl | 451 (M-2HCl + H)⁺ |
| 5 | H | 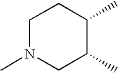 (3R, 4S) | 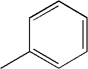 | CH₂ | 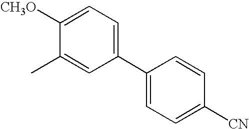 | 2HCl | 398 (M-2HCl + H)⁺ |
| 6 | H | 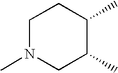 (3R, 4S) | 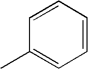 | CH₂ | 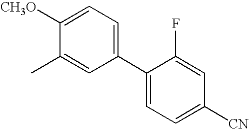 | 2HCl | 416 (M-2HCl + H)⁺ |
| 7 | 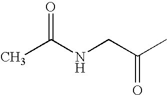 | 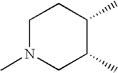 (3R, 4S) | 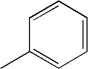 | CH₂ | 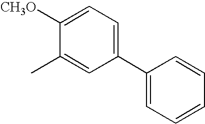 | | 472 (M + H)⁺ |
| 8 | 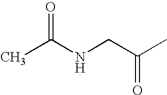 | 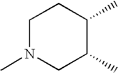 (3R, 4S) | 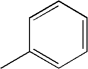 | CH₂ | 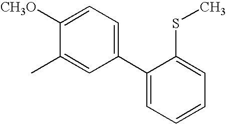 | HCl | 518 (M-HCl + H)⁺ |
| 9 | 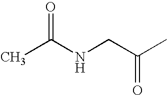 | 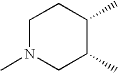 (3R, 4S) | 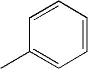 | CH₂ | 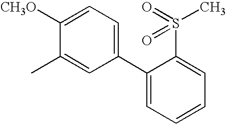 | HCl | 550 (M-HCl + H)⁺ |
| 10 | 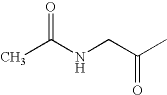 | 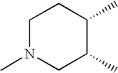 (3R, 4S) | 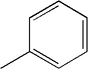 | CH₂ | 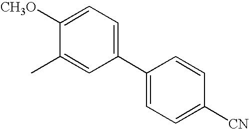 | | 497 (M + H)⁺ |
| 11 | 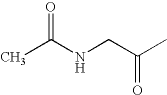 | 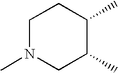 (3R, 4S) | 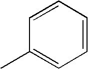 | CH₂ | 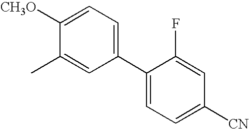 | | 515 (M + H)⁺ |
| 12 | 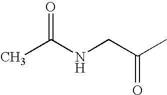 | 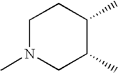 (3R, 4S) | 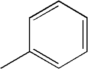 | CH₂ | 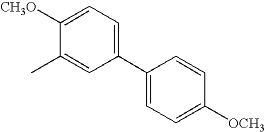 | | 502 (M + H)⁺ |

TABLE 5

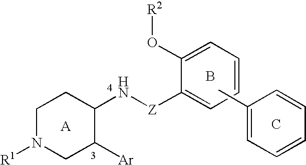

| Ex. No. | R¹ | 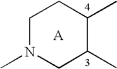 | Ar | Z | 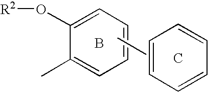 | Addi- tives | MS (ESI) |
|---|---|---|---|---|---|---|---|
| 13 | 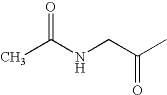 | 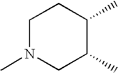 (3R, 4S) | 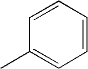 | CH₂ | 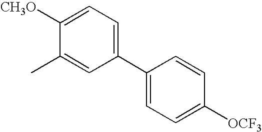 | | 556 (M + H)⁺ |
| 14 | 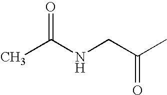 | 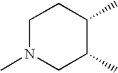 (3R, 4S) | 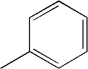 | CH₂ | 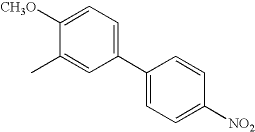 | | 517 (M + H)⁺ |
| 15 | 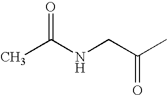 | 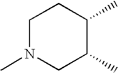 (3R, 4S) | 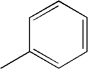 | CH₂ | 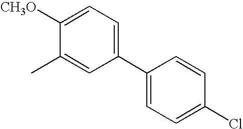 | | 506 (M + H)⁺ |
| 16 | 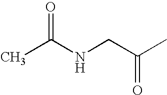 | 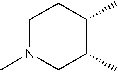 (3R, 4S) | 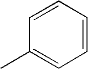 | CH₂ | 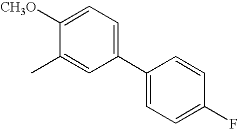 | | 490 (M + H)⁺ |
| 17 | 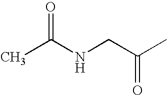 | 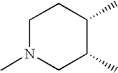 (3R, 4S) | 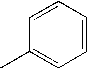 | CH₂ | 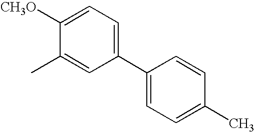 | | 486 (M + H)⁺ |
| 18 | 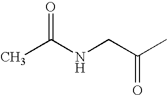 | 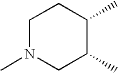 (3R, 4S) | 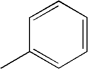 | CH₂ | 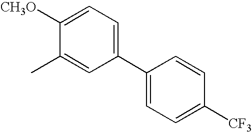 | | 540 (M + H)⁺ |
| 19 | 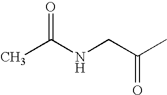 | 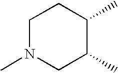 (3R, 4S) | 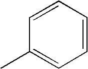 | CH₂ | 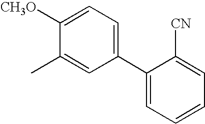 | | 497 (M + H)⁺ |
| 20 | 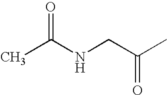 | 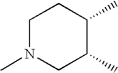 (3R, 4S) | 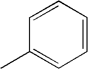 | CH₂ | 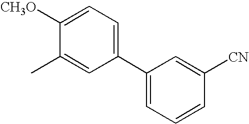 | | 497 (M + H)⁺ |
| 21 | 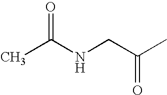 | 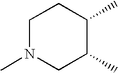 (3R, 4S) | 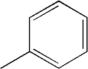 | CH₂ | 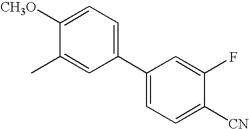 | | 515 (M + H)⁺ |
| 22 | 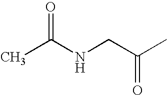 | 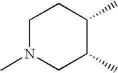 (3R, 4S) | 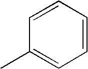 | CH₂ | 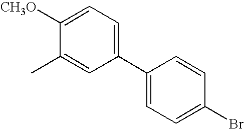 | | 550, 552 (M + H)⁺ |
| 23 | 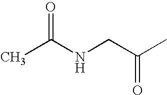 | 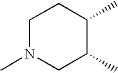 (3R, 4S) | 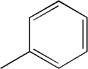 | CH₂ | 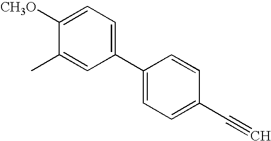 | | 496 (Ml + H)⁺ |
| 24 | 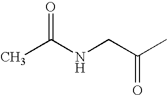 | 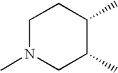 (3R, 4S) | 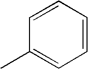 | CH₂ | 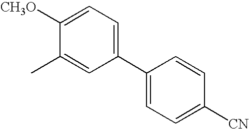 | HCl | 502 (M-HCl + H)⁺ |

TABLE 6

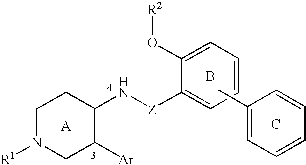

| Ex. No. | R¹ | 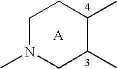 | Ar | Z | 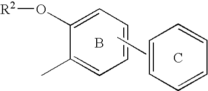 | Addi- tives | MS (ESI) |
|---|---|---|---|---|---|---|---|
| 25 | 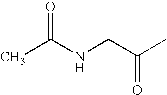 | 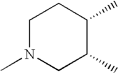 (3R, 4S) | 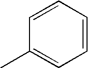 | CH₂ | 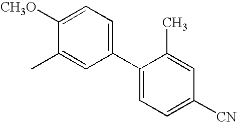 | HCl | 511 (M-HCl + H)⁺ |
| 26 | 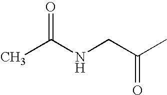 | 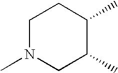 (3R, 4S) | 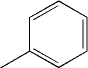 | CH₂ | 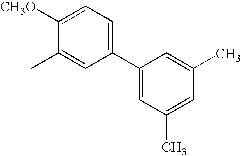 | HCl | 500 (M-HCl + H)⁺ |
| 27 | 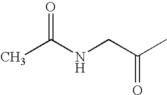 | 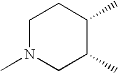 (3R, 4S) | 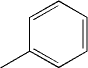 | CH₂ | 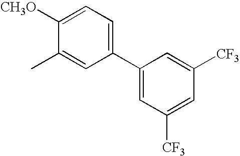 | | 608 (M + H)⁺ |
| 28 | 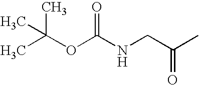 | 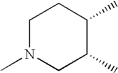 (3R, 4S) | 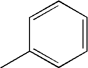 | CH₂ | 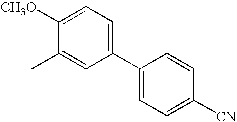 | | 455 (M-Boc + 2H)⁺ |
| 29 | 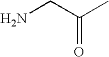 | 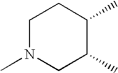 (3R, 4S) | 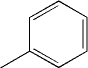 | CH₂ | 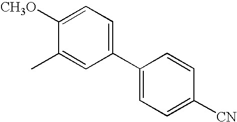 | 2HCl | 455 (M-2HCl + H)⁺ |
| 30 | 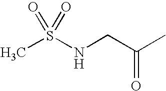 | 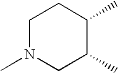 (3R, 4S) | 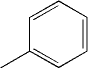 | CH₂ | 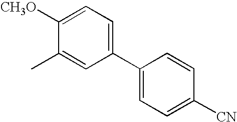 | | 533 (M + H)⁺ |
| 31 | 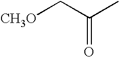 | 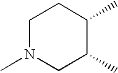 (3R, 4S) | 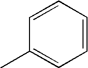 | CH₂ | 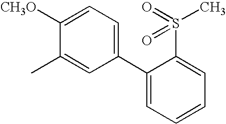 | HCl | 523 (M-HCl + H)⁺ |
| 32 | 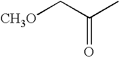 | 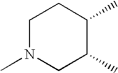 (3R, 4S) | 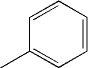 | CH₂ | 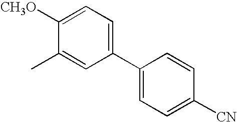 | | 470 (M + H)⁺ |
| 33 | 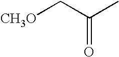 | 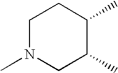 (3R, 4S) | 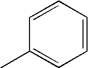 | CH₂ | 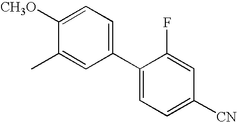 | | 488 (M + H)⁺ |
| 34 | 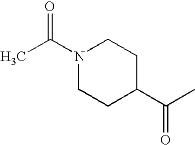 | 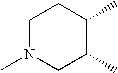 (3R, 4S) | 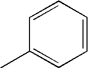 | CH₂ | 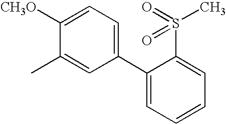 | HCl | 604 (M-HCl + H)⁺ |
| 35 | 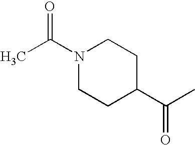 | 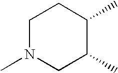 (3R, 4S) | 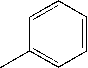 | CH₂ | 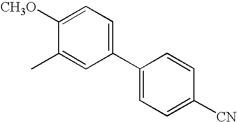 | | 551 (M + H)⁺ |
| 36 | 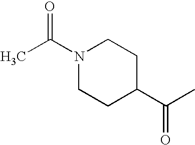 | 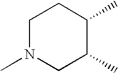 (3R, 4S) | 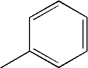 | CH₂ | 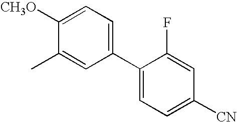 | | 569 (M + H)⁺ |

TABLE 7

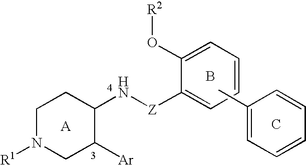

| Ex. No. | R¹ | 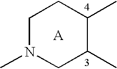 | Ar | Z | 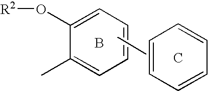 | Addi- tives | MS (ESI) |
|---|---|---|---|---|---|---|---|
| 37 | 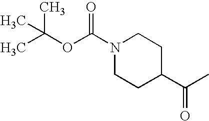 | 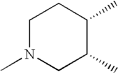 (3R, 4S) | 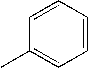 | CH₂ | 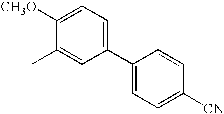 | | 509 (M-Boc + 2H)⁺ |
| 38 | 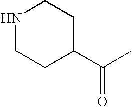 | 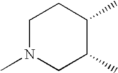 (3R, 4S) | 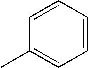 | CH₂ | 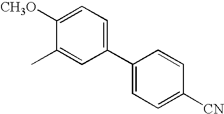 | 2HCl | 509 (M-2HCl + H)⁺ |
| 39 | 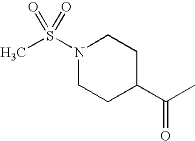 | 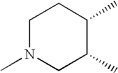 (3R, 4S) | 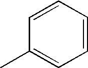 | CH₂ | 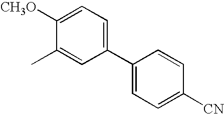 | | 587 (M + H)⁺ |
| 40 | 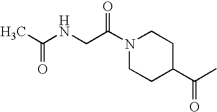 | 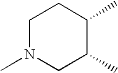 (3R, 4S) | 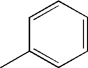 | CH₂ | 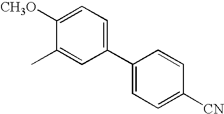 | HCl | 608 (M-HCl + H)⁺ |
| 41 | 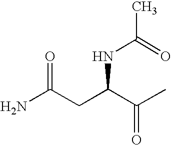 | 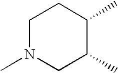 (3R, 4S) | 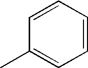 | CH₂ | 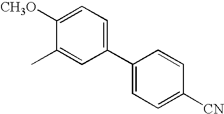 | | 554 (M + H)⁺ |
| 42 | 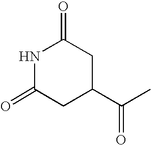 | 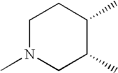 (3R, 4S) | 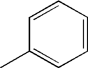 | CH₂ | 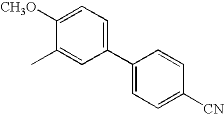 | | 537 (M + H)⁺ |
| 43 | 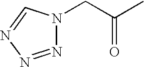 | 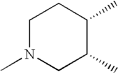 (3R, 4S) | 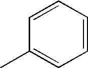 | CH₂ | 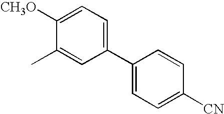 | | 508 (M + H)⁺ |
| 44 | 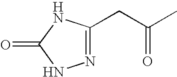 | 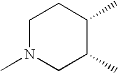 (3R, 4S) | 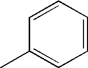 | CH₂ | 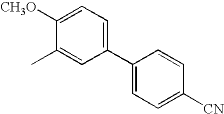 | | 523 (M + H)⁺ |
| 45 | 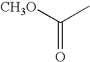 | 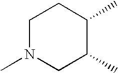 (3R, 4S) | 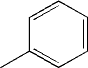 | CH₂ | 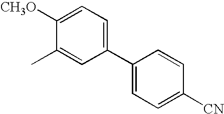 | | 456 (M + H)⁺ |
| 46 | 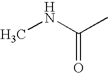 | 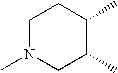 (3R, 4S) | 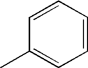 | CH₂ | 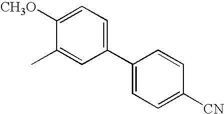 | | 455 (M + H)⁺ |

### Example 47

### N-[2-((3R,4S)-4-{[(4′-acetyl-4-methoxybiphenyl-3-yl)methyl]amino}-3-phenylpiperidin-1-yl)-2-oxoethyl]acetamide monohydrochloride

Using the compound obtained in Reference Example 1 and the compound obtained in Reference Example 10, the reaction and purification were performed in the same manner as in the method described in Example 7. The obtained product was treated with 1 equivalent of hydrogen chloride/ethyl acetate to give the title compound.

MS(ESI+): 514(M−HCl+H)

### Example 48

### N-{3′-[({(3R,4S)-1-[(acetylamino)acetyl]-3-phenylpiperidin-4-yl}amino)methyl]-4′-methoxybiphenyl-4-yl}acetamide monohydrochloride

Using the compound obtained in Reference Example 1 and the compound obtained in Reference Example 11, the reaction and purification were performed in the same manner as in the method described in Example 7. The obtained product was treated with 1 equivalent of hydrogen chloride/ethyl acetate to give the title compound.

MS(ESI+): 529(M−HCl+H)

### Example 49

### N-[2-((3R,4S)-4-{[(4′-tert-butyl-4-methoxybiphenyl-3-yl)methyl]amino}-3-phenylpiperidin-1-yl)-2-oxoethyl]acetamide monohydrochloride

Using the compound obtained in Reference Example 1 and the compound obtained in Reference Example 12, the reaction and purification were performed in the same manner as in the method described in Example 7. The obtained product was treated with 1 equivalent of hydrogen chloride/ethyl acetate to give the title compound.

MS(ESI+): 528(M−HCl+H)

### Example 50

### N-{2-[(3R,4S)-4-({[4′-(dimethylamino)-4-methoxybiphenyl-3-yl]methyl}amino)-3-phenylpiperidin-1-yl]-2-oxoethyl}acetamide dihydrochloride

Using the compound obtained in Reference Example 1 and the compound obtained in Reference Example 13, the reaction and purification were performed in the same manner as in the method described in Example 7. The obtained product was treated with 2 equivalents of hydrogen chloride/ethyl acetate to give the title compound.

MS(ESI+): 515(M−2HCl+H)

### Example 51

### N-{2-[(3R,4S)-4-({[4-methoxy-4′-(methylsulfonyl)biphenyl-3-yl]methyl}amino)-3-phenylpiperidin-1-yl]-2-oxoethyl}acetamide

Using the compound obtained in Reference Example 1 and the compound obtained in Reference Example 14, the title compound was obtained by the reaction and purification in the same manner as in the method described in Example 7.

MS(ESI+): 550(M+H)

### Example 52

### N-[2-((3R,4S)-4-{[(4′-cyano-5-fluoro-4-methoxybiphenyl-3-yl)methyl]amino}-3-phenylpiperidin-1-yl)-2-oxoethyl]acetamide

Using the compound obtained in Reference Example 1 and the compound obtained in Reference Example 15, the title compound was obtained by the reaction and purification in the same manner as in the method described in Example 7.

MS(ESI+): 515(M+H)

### Example 53

### methyl 3′-({[(3R,4S)-1-(N-acetylglycyl)-3-phenylpiperidin-4-yl]amino}methyl)-4′-methoxybiphenyl-4-carboxylate

Using the compound obtained in Reference Example 1 and the compound obtained in Reference Example 16, the title compound was obtained by the reaction and purification in the same manner as in the method described in Example 7.

MS(ESI+): 530(M+H)

### Example 54

### N-[2-((3R,4S)-4-{[(4′-chloro-4-methoxy-2′-methylbiphenyl-3-yl)methyl]amino}-3-phenylpiperidin-1-yl)-2-oxoethyl]acetamide

Using the compound obtained in Reference Example 1 and the compound obtained in Reference Example 17, the title compound was obtained by the reaction and purification in the same manner as in the method described in Example 7.

MS(ESI+): 520(M+H)

### Example 55

### N-{2-[(3R,4S)-4-({[4′-bromo-4-methoxy-3′-(trifluoromethyl)biphenyl-3-yl]methyl}amino)-3-phenylpiperidin-1-yl]-2-oxoethyl}acetamide

Using the compound obtained in Reference Example 1 and the compound obtained in Reference Example 18, the title compound was obtained by the reaction and purification in the same manner as in the method described in Example 7.

MS(ESI+): 618, 620(M+H)

### Example 56

### N-[2-((3R,4S)-4-{[(4′-cyano-2-fluoro-4-methoxybiphenyl-3-yl)methyl]amino}-3-phenylpiperidin-1-yl)-2-oxoethyl]acetamide

Using the compound obtained in Reference Example 1 and the compound obtained in Reference Example 19, the title compound was obtained by the reaction and purification in the same manner as in the method described in Example 7.

MS(ESI+): 515(M+H)

### Example 57

### N-{2-[(3R,4S)-4-({[4′-cyano-4-methoxy-2′-(trifluoromethyl)biphenyl-3-yl]methyl}amino)-3-phenylpiperidin-1-yl]-2-oxoethyl}acetamide

Using the compound obtained in Reference Example 1 and the compound obtained in Reference Example 20, the title compound was obtained by the reaction and purification in the same manner as in the method described in Example 7.

MS(ESI+): 565(M+H)

### Example 58

### 3′-({[(3R,4S)-1-(N-acetylglycyl)-3-phenylpiperidin-4-yl]amino}methyl)-4′-methoxybiphenyl-4-carboxamide

Using the compound obtained in Reference Example 1 and the compound obtained in Reference Example 21, the title compound was obtained by the reaction and purification in the same manner as in the method, described in Example 7.

MS(ESI+): 515(M+H)

### Example 59

### N-[2-((3R,4S)-4-{[(4′-cyano-6-fluoro-4-methoxybiphenyl-3-yl)methyl]amino}-3-phenylpiperidin-1-yl)-2-oxoethyl]acetamide

Using the compound obtained in Reference Example 1 and the compound obtained in Reference Example 22, the title compound was obtained by the reaction and purification in the same manner as in the method described in Example 7.

MS(ESI+): 515(M+H)

### Example 60

### N-[2-((3R,4S)-4-{[(4′-cyano-4-methoxy-6-methylbiphenyl-3-yl)methyl]amino}-3-phenylpiperidin-1-yl)-2-oxoethyl]acetamide

Using the compound obtained in Reference Example 1 and the compound obtained in Reference Example 23, the title compound was obtained by the reaction and purification in the same manner as in the method described in Example 7.

MS(ESI+): 511(M+H)

### Example 61

### N-[2-((3R,4S)-4-{[(4′-cyano-4-hydroxybiphenyl-3-yl)methyl]amino}-3-phenylpiperidin-1-yl)-2-oxoethyl]acetamide

Using the compound obtained in Reference Example 1 and the compound obtained in Reference Example 24, the title compound was obtained by the reaction and purification in the same manner as in the method described in Example 7.

MS(ESI+): 483(M+H)

### Example 62

### N-[2-((3R,4S)-4-{[(4′-cyano-4-methoxy-2-methylbiphenyl-3-yl)methyl]amino}-3-phenylpiperidin-1-yl)-2-oxoethyl]acetamide

Using the compound obtained in Reference Example 1 and the compound obtained in Reference Example 25, the title compound was obtained by the reaction and purification in the same manner as in the method described in Example 7.

MS(ESI+): 511(M+H)

### Example 63

### N-[2-((3R,4S)-4-{[1-(4′-cyano-4-methoxybiphenyl-3-yl)ethyl]amino}-3-phenylpiperidin-1-yl)-2-oxoethyl]acetamide (diastereomeric mixture)

To a solution of the compound (1.86 g) obtained in Reference Example 1, the compound (0.63 g) obtained in Reference Example 38 and Et₃N (0.76 g) in methylene chloride (60 mL) was added a solution of titanium tetrachloride (0.24 g) in methylene chloride (10 mL) at room temperature, and the mixture was stirred for 2 hr. The reaction mixture was concentrated under reduced pressure, and the residue was dissolved in methanol (15 mL). NaBH₃CN (0.47 g) was added to the reaction solution at room temperature, and the mixture was stirred for 30 min. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (solvent gradient; 50→100% ethyl acetate/hexane) to give the title compound (1.09 g, 43%) as a colorless amorphous solid.

MS(ESI+): 511(M+H)

### Example 64

### N-[2-((3R,4S)-4-{[1-(4′-cyano-4-methoxybiphenyl-3-yl)ethyl]amino}-3-phenylpiperidin-1-yl)-2-oxoethyl]acetamide (shorter retention time)

The diastereomeric mixture (0.40 g) obtained in Example 63 was purified by chiral column chromatography. Concentration of a fraction with a shorter retention time gave the title compound (0.22 g) as a colorless amorphous solid.

MS(ESI+): 511(M+H) [α]_{D}²⁵ +46.1° (c 0.51, MeOH) Diastereomer excess: 99.7% de

Purification Conditions by Chiral Column Chromatography

Column: CHIRALPAK AD 50 mmID×500 mmL
Solvent: hexane/IPE/diethylamine=75/25/0.2
Flow rate: 80 mL/min
Temperature: 40° C.
Detection method: UV 260 nm

### Example 65

### N-[2-((3R,4S)-4-{[1-(4′-cyano-4-methoxybiphenyl-3-yl)ethyl]amino}-3-phenylpiperidin-1-yl)-2-oxoethyl]acetamide (longer retention time)

In the purification by chiral column chromatography described in Example 64, concentration of a fraction with a longer retention time gave the title compound (0.086 g) as a colorless amorphous solid.

MS(ESI+): 511(M+H) [α]_{D}²⁵ −5.6° (c 0.51, MeOH) Diastereomer excess: 99.7% de

Purification Conditions by Chiral Column Chromatography

The same conditions as described in Example 64.

### Example 66

### N-{2-[(3R,4S)-4-({[5-(4-cyanophenyl)-2,3-dihydro-1-benzofuran-7-yl]methyl}amino)-3-phenylpiperidin-1-yl]-2-oxoethyl}acetamide

Using the compound obtained in Reference Example 1 and the compound obtained in Reference Example 26, the title compound was obtained by the reaction and purification in the same manner as in the method described in Example 7.

MS(ESI+): 509(M+H)

### Example 67

### N-{2-[(3R,4S)-4-({[4′-cyano-4-(2,2,2-trifluoroethoxy)biphenyl-3-yl]methyl}amino)-3-phenylpiperidin-1-yl]-2-oxoethyl}acetamide monohydrochloride

Using the compound obtained in Reference Example 1 and the compound obtained in Reference Example 27, the reaction and purification were performed in the same manner as in the method described in Example 7. The obtained product was treated with 1 equivalent of hydrogen chloride/ethyl acetate to give the title compound.

MS(ESI+): 565(M−HCl+H)

### Example 68

### N-{2-[(3R,4S)-4-({[4′-cyano-2′-fluoro-4-(trifluoromethoxy)biphenyl-3-yl]methyl}amino)-3-phenylpiperidin-1-yl]-2-oxoethyl}acetamide

Using the compound obtained in Reference Example 1 and the compound obtained in Reference Example 28, the title compound was obtained by the reaction and purification in the same manner as in the method described in Example 7.

MS(ESI+): 569(M+H)

### Example 69

### N-{2-[(3R,4S)-4-({[4′-cyano-2′-methyl-4-(trifluoromethoxy)biphenyl-3-yl]methyl}amino)-3-phenylpiperidin-1-yl]-2-oxoethyl}acetamide

Using the compound obtained in Reference Example 1 and the compound obtained in Reference Example 39, the title compound was obtained by the reaction and purification in the same manner as in the method described in Example 7.

MS(ESI+): 565(M+H)

### Example 70

### N-{2-[(3R,4S)-4-({[4′-chloro-2′-fluoro-4-(trifluoromethoxy)biphenyl-3-yl]methyl}amino)-3-phenylpiperidin-1-yl]-2-oxoethyl}acetamide

Using the compound obtained in Reference Example 1 and the compound obtained in Reference Example 29, the title compound was obtained by the reaction and purification in the same manner as in the method described in Example 7.

MS(ESI+): 578(M+H)

### Example 71

### N-{2-oxo-2-[(3R,4S)-3-phenyl-4-({[4-(trifluoromethoxy)-4′-(trifluoromethyl)biphenyl-3-yl]methyl}amino)piperidin-1-yl]ethyl}acetamide

Using the compound obtained in Reference Example 1 and the compound obtained in Reference Example 30, the title compound was obtained by the reaction and purification in the same manner as in the method described in Example 7.

MS(ESI+): 594(M+H)

### Example 72

### N-{2-[(3R,4S)-4-({[4′-chloro-4-(trifluoromethoxy)biphenyl-3-yl]methyl}amino)-3-phenylpiperidin-1-yl]-2-oxoethyl}acetamide

A solution of the compound (557 mg) obtained in Reference Example 1, the compound (451 mg) obtained in Reference Example 31, Et₃N (304 mg) and acetic acid (0.1 mL) in ethyl acetate (8 mL) was stirred at 45° C. for 10 min. NaBH(OAc)₃ (954 mg) was added to the reaction mixture, and the mixture was stirred at room temperature for 5 hr. Water was added to the reaction mixture, and the mixture was extracted with ethyl acetate. The organic layer was washed with a saturated aqueous sodium hydrogen carbonate solution and then with brine, dried over anhydrous magnesium sulfate and concentrated. The residue was purified by silica gel column chromatography (NH Chromatorex) (solvent gradient; 0→25% ethyl acetate/hexane) to give the title compound (128 mg, 15%) as a white amorphous solid.

Elemental analysis: C₂₉H₂₉ClF₃N₃O₃.H₂O Found C, 60.66; H, 5.19; N, 7.10; Calcd. C, 60.26; H, 5.41; N, 7.27. MS(ESI+): 560(M+H)

### Example 73

### N-{2-[(3R,4S)-4-({[4′-fluoro-4-(trifluoromethoxy)biphenyl-3-yl]methyl}amino)-3-phenylpiperidin-1-yl]-2-oxoethyl}acetamide

Using the compound obtained in Reference Example 1 and the compound obtained in Reference Example 32, the title compound was obtained by the reaction and purification in the same manner as in the method described in Example 7.

MS(ESI+): 544 (M+H)

### Example 74

### N-{2-[(3R,4S)-4-({[2′,4′-dichloro-4-(trifluoromethoxy)biphenyl-3-yl]methyl}amino)-3-phenylpiperidin-1-yl]-2-oxoethyl}acetamide

Using the compound obtained in Reference Example 1 and the compound obtained in Reference Example 33, the title compound was obtained by the reaction and purification in the same manner as in the method described in Example 7.

MS(ESI+): 594(M+H)

### Example 75

### N-{2-[(3R,4S)-4-({[2′,4′-difluoro-4-(trifluoromethoxy)biphenyl-3-yl]methyl}amino)-3-phenylpiperidin-1-yl]-2-oxoethyl}acetamide

Using the compound obtained in Reference Example 1 and the compound obtained in Reference Example 34, the title compound was obtained by the reaction and purification in the same manner as in the method described in Example 7.

MS(ESI+): 562(M+H)

### Example 76

### N-{2-[(3R,4S)-4-({[2′-chloro-4′-cyano-4-(trifluoromethoxy)biphenyl-3-yl]methyl}amino)-3-phenylpiperidin-1-yl]-2-oxoethyl}acetamide

Using the compound obtained in Reference Example 1 and the compound obtained in Reference Example 35, the title compound was obtained by the reaction and purification in the same manner as in the method described in Example 7.

MS(ESI+): 585(M+H)

### Example 77

### N-{2-[(3R,4S)-4-({[2′-chloro-4′-fluoro-4-(trifluoromethoxy)biphenyl-3-yl]methyl}amino)-3-phenylpiperidin-1-yl]-2-oxoethyl}acetamide

Using the compound obtained in Reference Example 1 and the compound obtained in Reference Example 36, the title compound was obtained by the reaction and purification in the same manner as in the method described in Example 7.

MS(ESI+): 578(M+H)

### Example 78

### N-{2-[(3R,4S)-4-({[4′-cyano-4-(difluoromethoxy)biphenyl-3-yl]methyl}amino)-3-phenylpiperidin-1-yl]-2-oxoethyl}acetamide

Using the compound obtained in Reference Example 1 and the compound obtained in Reference Example 37, the title compound was obtained by the reaction and purification in the same manner as in the method described in Example 7.

MS(ESI+): 533(M+H)

### Example 79

### 2-((3R,4S)-4-{[(4′-cyano-4-methoxybiphenyl-3-yl)methyl]amino}-3-phenylpiperidin-1-yl)-2-oxoethyl acetate

To a solution of the compound (155 mg) obtained in Example 5, Et₃N (184 μL) and acetoxyacetic acid (47 mg) in THF (5 mL) were added WSC.HCl (97 mg) and HOBt.H₂O (76 mg), and the mixture was stirred at room temperature for 16 hr. The reaction mixture was poured into water, and the product was extracted with ethyl acetate. The organic layer was washed with a saturated aqueous sodium hydrogen carbonate solution and brine and-dried, and the solvent was evaporated under reduced pressure. The obtained residue was purified by silica gel column chromatography (solvent gradient; 20→75% ethyl acetate/hexane) to give the title compound (62 mg, 38%) as white crystals.

MS(ESI+): 498(M+H)

### Example 80

### 3′-({[(3R,4S)-1-glycoloyl-3-phenylpiperidin-4-yl]amino}methyl)-4′-methoxybiphenyl-4-carbonitrile

Using the compound obtained in Example 5 and glycolic acid, the title compound was obtained by the reaction and purification in the same manner as in the method described in Example 79.

MS(ESI+): 456(M+H)

### Example 81

### 4-((3R,4S)-4-{[(4′-cyano-4-methoxybiphenyl-3-yl)methyl]amino}-3-phenylpiperidin-1-yl)-4-oxobutanamide

Using the compound obtained in Example 5 and succinamic acid, the title compound was obtained by the reaction and purification in the same manner as in the method described in Example 79.

MS(ESI+): 497(M+H)

### Example 82

### 3′-({[(3R,4S)-1-acetyl-3-phenylpiperidin-4-yl]amino}methyl)-4′-methoxybiphenyl-4-carbonitrile monohydrochloride

To a solution of the compound (172 mg) obtained in Example 5 and Et₃N (204 μL) in THF (5 mL) was added acetyl chloride (30 μL) at 0° C., and the mixture was stirred at room temperature for 2 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (50% ethyl acetate/hexane). The obtained product was treated with 1 equivalent of hydrogen chloride/ethyl acetate to give the title compound.

MS(ESI+): 440(M−HCl+H)

### Example 83

### 4′-methoxy-3′-({[(3R,4S)-1-(methylsulfonyl)-3-phenylpiperidin-4-yl]amino}methyl)biphenyl-4-carbonitrile

To a solution of the compound (177 mg) obtained in Example 5 and Et₃N (210 μL) in THF (5 mL) was added methanesulfonyl chloride (33 μL) at 0° C., and the mixture was stirred at room temperature for 2 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (50% ethyl acetate/hexane) to give the title compound.

MS(ESI+): 476(M+H)

### Example 84

### 4′-methoxy-3′-{[((3R,4S)-3-phenyl-1-{[1-(1H-tetrazol-1-ylacetyl)piperidin-4-yl]carbonyl}piperidin-4-yl)amino]methyl}biphenyl-4-carbonitrile monohydrochloride

Using the compound obtained in Example 38 and 1H-tetrazol-1-ylacetic acid, the reaction and purification were performed in the same manner as in the method described in Example 79. The obtained product was treated with 1 equivalent of hydrogen chloride/ethyl acetate to give the title compound.

MS(ESI+): 619(M−HCl+H)

### Example 85

### 4′-methoxy-3′-({[(3R,4S)-1-({1-[(5-oxo-4,5-dihydro-1H-1,2,4-triazol-3-yl)acetyl]piperidin-4-yl}carbonyl)-3-phenylpiperidin-4-yl]amino}methyl)biphenyl-4-carbonitrile monohydrochloride

Using the compound obtained in Example 38 and (5-oxo-4,5-dihydro-1H-1,2,4-triazol-3-yl)acetic acid, the reaction and purification were performed in the same manner as in the method described in Example 79. The obtained product was treated with 1 equivalent of hydrogen chloride/ethyl acetate to give the title compound.

MS(ESI+): 634(M−HCl+H)

### Example 86

### 4-[{(3R,4S)-4-{[(4′-cyano-4-methoxybiphenyl-3-yl)methyl]amino}-3-phenylpiperidin-1-yl}carbonyl]-N,N-dimethylpiperidine-1-carboxamide

Using the compound obtained in Example 38 and dimethylcarbamoyl chloride, the title compound was obtained by the reaction and purification in the same manner as in the method described in Example 83.

MS(ESI+): 580(M+H)

### Example 87

### 4′-methoxy-3′-{[((3R,4S)-3-phenyl-1-{[1-(1H-1,2,4-triazol-1-ylacetyl)piperidin-4-yl]carbonyl}piperidin-4-yl)amino]methyl}biphenyl-4-carbonitrile monohydrochloride

Using the compound obtained in Example 38 and (1H-1,2,4-triazol-1-yl)acetic acid, the reaction and purification were performed in the same manner as in the method described in Example 79. The obtained product was treated with 1 equivalent of hydrogen chloride/ethyl acetate to give the title compound.

MS(ESI+): 618(M−HCl+H)

### Example 88

### 3′-[({(3R,4S)-1-[(5,5-dimethyl-2,4-dioxo-1,3-oxazolidin-3-yl)acetyl]-3-phenylpiperidin-4-yl}amino)methyl]-4′-methoxybiphenyl-4-carbonitrile

Using the compound obtained in Example 5 and (5,5-dimethyl-2,4-dioxo-1,3-oxazolidin-3-yl)acetic acid synthesized by a known method (WO2006/030975), the title compound was obtained by the reaction and purification in the same manner as in the method described in Example 79.

MS(ESI+): 567(M+H)

### Example 89

### tert-butyl (3R,4S)-4-{[(4′-cyano-4-methoxybiphenyl-3-yl)methyl]amino}-3-(4-fluorophenyl)piperidine-1-carboxylate

(Step 1)

A solution of tert-butyl 3-(4-fluorophenyl)-4-oxopiperidine-1-carboxylate (6.0 g) synthesized by a known method (WO03/101964), (S)-1-phenylethylamine (3.7 g) and aluminum chloride (0.14 g) in toluene (40 mL) was stirred for 14 hr under an argon atmosphere, while azeotropic dehydration was carried out at 140° C. using a Dean-Stark trap, and the reaction mixture was concentrated under reduced pressure. Raney-nickel (10 g) was washed with ethanol and suspended in ethanol (45 mL). The residue obtained from the reaction mixture mentioned above was added, and the mixture was stirred under a hydrogen pressure of 0.5 MPa at 25° C. for 48 hr. Raney-nickel was removed by decantation, and the supernatant was concentrated under reduced pressure. The residue was purified by silica gel column chromatography (solvent gradient; 10→50% ethyl acetate/hexane) to give a pale-yellow oil (6.1 g). A mixture of the obtained residue, palladium carbon (5 wt %, 1 g) and ethanol (50 mL) was stirred under a hydrogen pressure of 0.5 MPa at 60° C. for 30 hr. The palladium carbon was filtered off, and the filtrate was concentrated under reduced pressure. The residue was purified by silica gel column chromatography (solvent gradient; hexane:ethyl acetate=2:1→ethyl acetate alone→ethyl acetate:methanol=2:1) to give tert-butyl (3R,4S)-4-amino-3-(4-fluorophenyl)piperidine-1-carboxylate (4.3 g, 71%) as a colorless powder.

Elemental analysis: C₁₆H₂₃FN₂O₂ Found C, 65.36; H, 7.77; N, 9.34; Calcd. C, 65.28; H, 7.88; N, 9.52.

(Step 2)

To a solution of the compound (1.2 g) obtained in Step 1, 3′-formyl-4′-methoxybiphenyl-4-carbonitrile (0.95 g) and acetic acid (0.8 mL) in methylene chloride (30 mL) was added NaBH(OAc)₃ (2.5 g), and the mixture was stirred at room temperature for 4 hr. A saturated aqueous sodium hydrogen carbonate solution was added to the reaction mixture, and the mixture was extracted with ethyl acetate. The organic layer was washed with a saturated aqueous sodium hydrogen carbonate solution and then with brine, dried over anhydrous magnesium sulfate and concentrated. The residue was purified by silica gel column chromatography (NH Chromatorex) (solvent gradient; 0→50% ethyl acetate/hexane) to give the title compound (1.9 g, 81%) as white crystals.

Elemental analysis: C₃₁H₃₄FN₃O₃.H₂O Found C, 71.05; H, 6.68; N, 8.21; Calcd. C, 70.97; H, 6.72; N, 8.01. MS(ESI+): 516(M+H)

### Example 90

### 3′-({[(3R,4S)-3-(4-fluorophenyl)piperidin-4-yl]amino}methyl)-4′-methoxybiphenyl-4-carbonitrile dihydrochloride

Using the compound obtained in Example 89, the title compound was obtained by the reaction and purification in the same manner as in the method described in Example 4.

MS(ESI+): 416(M−2HCl+H)

### Example 91

### N-{2-[(3R,4S)-4-{[(4′-cyano-4-methoxybiphenyl-3-yl)methyl]amino}-3-(4-fluorophenyl)piperidin-1-yl]-2-oxoethyl}acetamide

Using the compound obtained in Example 90 and acetylglycine, the title compound was obtained by the reaction and purification in the same manner as in the method described in Example 79.

MS (ESI+): 515 (M+H)

### Example 92

### 3′-({[(3R,4S)-1-[(1-acetylpiperidin-4-yl)carbonyl]-3-(4-fluorophenyl)piperidin-4-yl]amino}methyl)-4′-methoxybiphenyl-4-carbonitrile

Using the compound obtained in Example 90 and 1-acetylpiperidine-4-carboxylic acid, the title compound was obtained by the reaction and purification in the same manner as in the method described in Example 79.

MS(ESI+): 569(M+H)

### Example 93

### tert-butyl (3R,4S)-4-({[4′-cyano-2′-fluoro-4-(trifluoromethoxy)biphenyl-3-yl]methyl}amino)-3-phenylpiperidine-1-carboxylate

Using tert-butyl (3R,4S)-4-amino-3-phenylpiperidine-1-carboxylate and the compound obtained in Reference Example 28, the title compound was obtained by the reaction and purification in the same manner as in the method described in Example 7.

MS(ESI+): 570(M+H)

### Example 94

### 2-fluoro-3′-({[(3R,4S)-3-phenylpiperidin-4-yl]amino}methyl)-4′-(trifluoromethoxy)biphenyl-4-carbonitrile dihydrochloride

Using the compound obtained in Example 93, the title compound was obtained by the reaction and purification in the same manner as in the method described in Example 4.

MS(ESI+): 470(M−2HCl+H)

### Example 95

### 2-fluoro-3′-({[(3R,4S)-3-phenylpiperidin-4-yl]amino}methyl)-4′-(trifluoromethoxy)biphenyl-4-carbonitrile bis(trifluoroacetate)

A solution of the compound (4.1 g) obtained in Example 93 in TFA (10 mL) was stirred at room temperature for 2 hr. The reaction solution was concentrated under reduced pressure to give the title compound.

MS(ESI+): 470(M−2TFA+H)

### Example 96

### 3′-[({(3R,4S)-1-[(5,5-dimethyl-2,4-dioxo-1,3-oxazolidin-3-yl)acetyl]-3-phenylpiperidin-4-yl}amino)methyl]-2-fluoro-4′-(trifluoromethoxy)biphenyl-4-carbonitrile

To a solution of the compound (325 mg) obtained in Example 94, Et₃N (182 mg) and (5,5-dimethyl-2,4-dioxo-1,3-oxazolidin-3-yl)acetic acid (168 mg) in DMF (6 mL) were added WSC.HCl (173 mg) and HOBt.H₂O (138 mg), and the mixture was stirred at room temperature for 12 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (NH Chromatorex) (solvent gradient; 20→75% ethyl acetate/hexane) to give the title compound (180 mg, 47%) as a colorless amorphous solid.

Elemental analysis: C₃₃H₃₀F₄N₄O_{5.}0.3H₂O Found C, 61.63; H, 4.80; N, 8.72; Calcd. C, 61.54; H, 4.79; N, 8.70. MS(ESI+): 639(M+H)

### Example 97

### 3′-[({(3R,4S)-1-[(5,5-dimethyl-2,4-dioxo-1,3-oxazolidin-3-yl)acetyl]-3-phenylpiperidin-4-yl}amino)methyl]-2-fluoro-4′-(trifluoromethoxy)biphenyl-4-carbonitrile monohydrochloride

The compound (78 mg) obtained in Example 96 was treated with 1 equivalent of 4N hydrogen chloride/ethyl acetate (0.1 mL), and crystallized from ethyl acetate/IPE to give the title compound.

Melting point: 198-200° C. Elemental analysis: C₃₃H₃₁ClF₄N₄O₅.0.5H₂O Found C, 58.02; H, 4.58; N, 8.16; Calcd. C, 57.94; H, 4.71; N, 8.19. MS(ESI+): 639(M−HCl+H)

### Example 98

### 3′-{[((3R,4S)-1-{[(4S)-2,5-dioxoimidazolidin-4-yl]acetyl}-3-phenylpiperidin-4-yl)amino]methyl}-2-fluoro-4′-(trifluoromethoxy)biphenyl-4-carbonitrile

Using the compound obtained in Example 94 and [(4S)-2,5-dioxoimidazolidin-4-yl]acetic acid synthesized by a known method (Journal of the American Chemical Society (1947), 69, 1382), the title compound was obtained by the reaction and purification in the same manner as in the method described in Example 79.

MS (ESI+): 610(M+H)

### Example 99

### 3′-[({(3R,4S)-1-[(2,5-dioxoimidazolidin-1-yl)acetyl]-3-phenylpiperidin-4-yl}amino)methyl]-2-fluoro-4′-(trifluoromethoxy)biphenyl-4-carbonitrile

Using the compound obtained in Example 94 and (2,5-dioxoimidazolidin-1-yl)acetic acid synthesized by a known method (Journal of the Chemical Society, Perkin Transactions 1: Organic and Bio-Organic Chemistry (1972-1999) (1988), (12), 3175-82), the title compound was obtained by the reaction and purification in the same manner as in the method described in Example 79.

MS(ESI+): 610(M+H)

### Example 100

### 2-fluoro-3′-({[(3R,4S)-1-glycoloyl-3-phenylpiperidin-4-yl]amino}methyl)-4′-(trifluoromethoxy)biphenyl-4-carbonitrile

To a solution of the compound (325 mg) obtained in Example 94, Et₃N (182 mg) and glycolic acid (92 mg) in DMF (6 mL) were added WSC.HCl (173 mg) and HOBt.H₂O (138 mg), and the mixture was stirred at room temperature for 12 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (NH Chromatorex) (solvent gradient; 20→75% ethyl acetate/hexane) to give the title compound (215 mg, 68%) as white crystals.

Melting point: 132-134° C. Elemental analysis: C₂₈H₂₅F₄N₃O₃ Found C, 63.72; H, 4.84; N, 7.84; Calcd. C, 63.75; H, 4.78; N, 7.97. MS(ESI+): 528(M+H)

### Example 101

### 3′-[({(3R,4S)-1-[(1-acetylpiperidin-4-yl)carbonyl]-3-phenylpiperidin-4-yl}amino)methyl]-2-fluoro-4′-(trifluoromethoxy)biphenyl-4-carbonitrile

To a solution of the compound (325 mg) obtained in Example 94, Et₃N (182 mg) and 1-acetylpiperidine-4-carboxylic acid (154 mg) in DMF (6 mL) were added WSC.HCl (173 mg) and HOBt.H₂O (138 mg), and the mixture was stirred at room temperature for 12 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (NH Chromatorex) (solvent gradient; 20→75% ethyl acetate/hexane) to give the title compound (253 mg, 68%) as a colorless amorphous solid.

Elemental analysis: C₃₄H₃₄F₄N₄O₃.0.2H₂O Found C, 65.29; H, 5.70; N, 8.54; Calcd. C, 65.21; H, 5.54; N, 8.95. MS(ESI+): 623(M+H)

### Example 102

### 3′-[({(3R,4S)-1-[(2,6-dioxopiperidin-4-yl)carbonyl]-3-phenylpiperidin-4-yl}amino)methyl]-2-fluoro-4′-(trifluoromethoxy)biphenyl-4-carbonitrile

To a solution of the compound (325 mg) obtained in Example 94, Et₃N (182 mg) and 2,6-dioxopiperidine-4-carboxylic acid (141 mg) in DMF (6 mL) were added WSC.HCl (173 mg) and HOBt.H₂O (138 mg), and the mixture was stirred at room temperature for 12 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (NH Chromatorex) (solvent gradient; 20→75% ethyl acetate/hexane) to give the title compound (245 mg, 67%) as a colorless amorphous solid.

Elemental analysis: C₃₂H₂₈F₄N₄O₄ Found C, 62.78; H, 4.94; N, 8.92; Calcd. C, 63.15; H, 4.64; N, 9.21. MS(ESI+): 609(M+H)

### Example 103

### 3′-[({(3R,4S)-1-[(2,4-dioxo-1,3-oxazolidin-3-yl)acetyl]-3-phenylpiperidin-4-yl}amino)methyl]-2-fluoro-4′-(trifluoromethoxy)biphenyl-4-carbonitrile

Using the compound obtained in Example 94 and (2,4-dioxo-1,3-oxazolidin-3-yl)acetic acid, the title compound was obtained by the reaction and purification in the same manner as in the method described in Example 79.

MS(ESI+): 611(M+H)

### Example 104

### 2-[(3R,4S)-4-({[4′-cyano-2′-fluoro-4-(trifluoromethoxy)biphenyl-3-yl]methyl}amino)-3-phenylpiperidin-1-yl]-2-oxoacetamide

To a solution of the compound (380 mg) obtained in Example 94, Et₃N (213 mg) and oxamic acid (94 mg) in DMF (6 mL) were added WSC.HCl (201 mg) and HOBt.H₂O (161 mg) and the mixture was stirred at room temperature for 13 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (solvent gradient; 50→100% ethyl acetate/hexane) to give the title compound (230 mg, 61%) as white crystals.

Melting point: 142-144° C. Elemental analysis: C₂₈H₂₄F₄N₄O₃ Found C, 62.09; H, 4.55; N, 10.27; Calcd. C, 62.22; H, 4.48; N, 10.37. MS(ESI+): 541(M+H)

### Example 105

### 2-[(3R,4S)-4-({[4′-cyano-2′-fluoro-4-(trifluoromethoxy)biphenyl-3-yl]methyl}amino)-3-phenylpiperidin-1-yl]-N,N-dimethyl-2-oxoacetamide

Using the compound obtained in Example 94 and N,N-dimethyloxamic acid, the title compound was obtained by the reaction and purification in the same manner as in the method described in Example 79.

MS(ESI+): 569(M+H)

### Example 106

### 3′-[({(3R,4S)-1-[(2,4-dioxo-3,4-dihydropyrimidin-1(2H)-yl)acetyl]-3-phenylpiperidin-4-yl}amino)methyl]-2-fluoro-4′-(trifluoromethoxy)biphenyl-4-carbonitrile

Using the compound obtained in Example 94 and (2,4-dioxo-3,4-dihydro-2H-pyrimidin-1-yl)acetic acid, the title compound was obtained by the reaction and purification in the same manner as in the method described in Example 79.

MS(ESI+): 622(M+H)

### Example 107

### 2-fluoro-3′-[({(3R,4S)-3-phenyl-1-[(3,4,4-trimethyl-2,5-dioxoimidazolidin-1-yl)acetyl]piperidin-4-yl}amino)methyl]-4′-(trifluoromethoxy)biphenyl-4-carbonitrile monohydrochloride

(Step 1)

To a solution of 1,5,5-trimethylhydantoin (4.98 g) in DMF (30 mL) was added 60% NaH (1.68 g) at 0° C., and the mixture was stirred for 30 min. A solution of benzyl bromoacetate (8.0 g) in DMF (10 mL) was added to the reaction mixture, and the mixture was stirred at room temperature for 3 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (solvent gradient; 50→100% ethyl acetate/hexane) to give a colorless oil (8.5 g). A mixture of the obtained oil (7.3 g), 10% palladium carbon (0.73 g) and ethanol (130 mL) was stirred at room temperature under a hydrogen atmosphere (0.1 MPa) for 5 hr. The catalyst was filtered off, and the filtrate was concentrated under reduced pressure to give (3,4,4-trimethyl-2,5-dioxoimidazolidin-1-yl)acetic acid (4.6 g) as white crystals.

Melting point: 130-132° C.

(Step 2)

Using the compound obtained in Example 94 and the compound obtained in Step 1, the reaction and purification were performed in the same manner as in the method described in Example 79. The obtained product was treated with 1 equivalent of hydrogen chloride/ethyl acetate to give the title compound.

MS(ESI+): 652(M−HCl+H)

### Example 108

### 2-[(3R,4S)-4-({[4′-cyano-2′-fluoro-4-(trifluoromethoxy)biphenyl-3-yl]methyl}amino)-3-phenylpiperidin-1-yl]-N-methyl-2-oxoacetamide monohydrochloride

Using the compound obtained in Example 94 and oxalic acid mono-(N-methyl)-amide, the reaction and purification were performed in the same manner as in the method described in Example 79. The obtained product was treated with 1 equivalent of hydrogen chloride/ethyl acetate to give the title compound. MS(ESI+): 555(M−HCl+H)

### Example 109

### 3′-[({(3R,4S)-1-[(2,6-dioxopiperidin-1-yl)acetyl]-3-phenylpiperidin-4-yl}amino)methyl]-2-fluoro-4′-(trifluoromethoxy)biphenyl-4-carbonitrile monohydrochloride

Using the compound obtained in Example 94 and (2,6-dioxopiperidin-1-yl)acetic acid, the reaction and purification were performed in the same manner as in the method described in Example 79. The obtained product was treated with 1 equivalent of hydrogen chloride/ethyl acetate to give the title compound. That is, the title compound was obtained by the following procedure.

A mixture of 2-fluoro-3′-({[(3R,4S)-3-phenylpiperidin-4-yl]amino}methyl)-4′-(trifluoromethoxy)biphenyl-4-carbonitrile dihydrochloride (332 mg), (2,6-dioxopiperidin-1-yl)acetic acid (128 mg), WSC.HCl (144 mg), HOBt.H₂O (115 mg), triethylamine (152 mg) and DMF (6 mL) was stirred at room temperature for 14 hr. The reaction mixture was poured into water, and the product was extracted with ethyl acetate. The organic layer was washed with a saturated aqueous sodium hydrogen carbonate solution and brine and dried over magnesium sulfate, and the solvent was evaporated under reduced pressure. The obtained residue was purified by silica gel column chromatography (solvent gradient; 50→100% ethyl acetate/hexane). The obtained product was treated with 4N hydrogen chloride/ethyl acetate and the mixture was concentrated under reduced pressure. The obtained residue was crystallized from diisopropyl ether/hexane/ethyl acetate to give the title compound (277 mg) as white powder.

Melting point: 223° C. Elemental analysis: C₃₃H₃₁ClF₄N₄O₄ Found C, 59.94; H, 4.75; N, 8.45; Calcd. C, 60.14; H, 4.74; N, 8.50. MS(ESI+): 623(M−HCl+H)

### Example 110

### (3R,4S)-4-({[4′-cyano-2′-fluoro-4-(trifluoromethoxy)biphenyl-3-yl]methyl}amino)-N-methyl-3-phenylpiperidine-1-carboxamide

Using the compound obtained in Example 94 and methyl isocyanate, the title compound was obtained by the reaction and purification in the same manner as in the method described in Example 83.

MS(ESI+): 527(M+H)

### Example 111

### 3′-[({(3R,4S)-1-[(4,4-dimethyl-2,5-dioxoimidazolidin-1-yl)acetyl]-3-phenylpiperidin-4-yl}amino)methyl]-2-fluoro-4′-(trifluoromethoxy)biphenyl-4-carbonitrile monohydrochloride

Using the compound obtained in Example 94 and (4,4-dimethyl-2,5-dioxoimidazolidin-1-yl)acetic acid, the reaction and purification were performed in the same manner as in the method described in Example 79. The obtained product was treated with 1 equivalent of hydrogen chloride/ethyl acetate to give the title compound.

MS(ESI+): 638(M−HCl+H)

### Example 112

### 2-fluoro-3′-[({(3R,4S)-1-[(3-oxo-2-azaspiro[4.5]dec-2-yl)acetyl]-3-phenylpiperidin-4-yl}amino)methyl]-4′-(trifluoromethoxy)biphenyl-4-carbonitrile monohydrochloride

(Step 1)

Using 2-azaspiro[4.5]decan-3-one, (3-oxo-2-azaspiro[4.5]dec-2-yl)acetic acid was obtained by the reaction and purification in the same manner as in the method described in Example 107, Step 1.

Melting point: 122-124° C.

(Step 2)

Using the compound obtained in Example 94 and the compound obtained in Step 1, the reaction and purification were performed in the same manner as in the method described in Example 79. The obtained product was treated with 1 equivalent of hydrogen chloride/ethyl acetate to give the title compound.

MS(ESI+): 663(M−HCl+H)

### Example 113

### 3′-[({(3R,4S)-1-[(2,4-dioxo-1,3-diazaspiro[4.4]non-3-yl)acetyl]-3-phenylpiperidin-4-yl}amino)methyl]-2-fluoro-4′-(trifluoromethoxy)biphenyl-4-carbonitrile monohydrochloride

Using the compound obtained in Example 94 and (2,4-dioxo-1,3-diazaspiro[4.4]non-3-yl)acetic acid, the reaction and purification were performed in the same manner as in the method described in Example 79. The obtained product was treated with 1 equivalent of hydrogen chloride/ethyl acetate to give the title compound.

MS(ESI+): 664(M−HCl+H)

### Example 114

### tert-butyl (3R,4S)-4-({[4′-chloro-4-(trifluoromethoxy)biphenyl-3-yl]methyl}amino)-3-phenylpiperidine-1-carboxylate

Using tert-butyl (3R,4S)-4-amino-3-phenylpiperidine-1-carboxylate and the compound obtained in Reference Example 31, the title compound was obtained by the reaction and purification in the same manner as in the method described in Example 7.

MS(ESI+): 561(M+H)

### Example 115

### (3R,4S)-N-{[4′-chloro-4-(trifluoromethoxy)biphenyl-3-yl]methyl}-3-phenylpiperidin-4-amine dihydrochloride

A solution of the compound (2.1 g) obtained in Example 114 in TFA (6 mL) was stirred at room temperature for 2 hr. The reaction solution was concentrated under reduced pressure, and the residue was dissolved in a mixture of ethyl acetate-1M NaOH aqueous solution. The organic layer was washed with brine and dried, and the solvent was evaporated under reduced pressure. The residue was treated with 2 equivalents of hydrogen chloride/ethyl acetate to give the title compound.

MS(ESI+): 461(M−2HCl+H)

### Example 116

### 3-{2-[(3R,4S)-4-({[4′-chloro-4-(trifluoromethoxy)biphenyl-3-yl]methyl}amino)-3-phenylpiperidin-1-yl]-2-oxoethyl}-5,5-dimethyl-1,3-oxazolidine-2,4-dione monohydrochloride

To a solution of the compound (267 mg) obtained in Example 115, Et₃N (152 mg) and (5,5-dimethyl-2,4-dioxo-1,3-oxazolidin-3-yl)acetic acid (140 mg) in DMF (6 mL) were added WSC.HCl (144 mg) and HOBt.H₂O (115 mg), and the mixture was stirred at room temperature for 14 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (solvent gradient; 50→100% ethyl acetate/hexane). The obtained product was treated with 1 equivalent of hydrogen chloride/ethyl acetate to give the title compound.

MS(ESI+): 630(M−HCl+H)

### Example 117

### 4-{[(3R,4S)-4-({[4′-chloro-4-(trifluoromethoxy)biphenyl-3-yl]methyl}amino)-3-phenylpiperidin-1-yl]carbonyl}piperidine-2,6-dione monohydrochloride

Using the compound obtained in Example 115 and 2,6-dioxopiperidine-4-carboxylic acid, the reaction and purification were performed in the same manner as in the method described in Example 79. The obtained product was treated with 1 equivalent of hydrogen chloride/ethyl acetate to give the title compound. That is, the title compound was obtained by the following procedure.

A mixture of (3R,4S)-N-{[4′-chloro-4-(trifluoromethoxy)biphenyl-3-yl]methyl}-3-phenylpiperidin-4-amine dihydrochloride (267 mg), 2,6-dioxopiperidine-4-carboxylic acid (118 mg), WSC.HCl (144 mg), HOBt.H₂O (115 mg), triethylamine (152 mg) and DMF (6 mL) was stirred at room temperature for 14 hr. The reaction mixture was poured into water, and the product was extracted with ethyl acetate. The organic layer was washed with a saturated aqueous sodium hydrogen carbonate solution and brine and dried over magnesium sulfate, and the solvent was evaporated under reduced pressure. The obtained residue was purified by silica gel column chromatography (solvent gradient; 20→50% ethyl acetate/hexane) to give 4-{[(3R,4S)-4-({[4′-chloro-4-(trifluoromethoxy)biphenyl-3-yl]methyl}amino)-3-phenylpiperidin-1-yl]carbonyl}piperidine-2,6-dione as an amorphous solid. The obtained amorphous solid was treated with 4N hydrogen chloride/ethyl acetate (0.3 mL) and the mixture was concentrated under reduced pressure. The obtained residue was crystallized from diisopropyl ether/hexane/ethyl acetate to give the title compound (197 mg) as white powder.

Melting point: 153° C. Elemental analysis: C₃₁H₃₀Cl₂F₃N₃O_{4.}0.5H₂O Found C, 57.75; H, 4.92; N, 6.50; Calcd. C, 57.68; H, 4.84; N, 6.51. MS(ESI+): 600(M−HCl+H)

### Example 118

### (3R,4S)-1-[(1-acetylpiperidin-4-yl)carbonyl]-N-{[4′-chloro-4-(trifluoromethoxy)biphenyl-3-yl]methyl}-3-phenylpiperidin-4-amine monohydrochloride

Using the compound obtained in Example 115 and 1-acetylpiperidine-4-carboxylic acid, the reaction and purification were performed in the same manner as in the method described in Example 79. The obtained product was treated with 1 equivalent of hydrogen chloride/ethyl acetate to give the title compound.

MS(ESI+): 614(M−HCl+H)

### Example 119

### tert-butyl (3R,4S)-4-({[2′-chloro-4′-cyano-4-(trifluoromethoxy)biphenyl-3-yl]methyl}amino)-3-phenylpiperidine-1-carboxylate

Using tert-butyl (3R,4S)-4-amino-3-phenylpiperidine-1-carboxylate and the compound obtained in Reference Example 35, the title compound was obtained by the reaction and purification in the same manner as in the method described in Example 7.

MS(ESI+): 586(M+H)

### Example 120

### 2-chloro-3′-({[(3R,4S)-3-phenylpiperidin-4-yl]amino}methyl)-4′-(trifluoromethoxy)biphenyl-4-carbonitrile dihydrochloride

Using the compound obtained in Example 119, the title compound was obtained by the reaction and purification in the same manner as in the method described in Example 115.

MS(ESI+): 486(M−2HCl+H)

### Example 121

### 2-chloro-3′-[({(3R,4S)-1-[(5,5-dimethyl-2,4-dioxo-1,3-oxazolidin-3-yl)acetyl]-3-phenylpiperidin-4-yl}amino)methyl]-4′-(trifluoromethoxy)biphenyl-4-carbonitrile monohydrochloride

Using the compound obtained in Example 120 and (5,5-dimethyl-2,4-dioxo-1,3-oxazolidin-3-yl)acetic acid, the reaction and purification were performed in the same manner as in the method described in Example 79. The obtained product was treated with 1 equivalent of hydrogen chloride/ethyl acetate to give the title compound.

MS(ESI+): 655(M−HCl+H)

### Example 122

### 2-chloro-3′-[({(3R,4S)-1-[(2,6-dioxopiperidin-4-yl)carbonyl]-3-phenylpiperidin-4-yl}amino)methyl]-4′-(trifluoromethoxy)biphenyl-4-carbonitrile monohydrochloride

Using the compound obtained in Example 120 and 2,6-dioxopiperidine-4-carboxylic acid, the reaction and purification were performed in the same manner as ′in the method described in Example 79. The obtained product was treated with 1 equivalent of hydrogen chloride/ethyl acetate to give the title compound.

MS(ESI+): 625(M−HCl+H)

### Example 123

### 3′-[({(3R,4S)-1-[(1-acetylpiperidin-4-yl)carbonyl]-3-phenylpiperidin-4-yl}amino)methyl]-2-chloro-4′-(trifluoromethoxy)biphenyl-4-carbonitrile monohydrochloride

Using the compound obtained in Example 120 and 1-acetylpiperidine-4-carboxylic acid, the reaction and purification were performed in the same manner as in the method described in Example 79. The obtained product was treated with 1 equivalent of hydrogen chloride/ethyl acetate to give the title compound.

MS(ESI+): 639(M−HCl+H)

### Example 124

### tert-butyl (3R,4S)-4-({[2′,4′-dichloro-4-(trifluoromethoxy)biphenyl-3-yl]methyl}amino)-3-phenylpiperidine-1-carboxylate

Using tert-butyl (3R,4S)-4-amino-3-phenylpiperidine-1-carboxylate and the compound obtained in Reference Example 33, the title compound was obtained by the reaction and purification in the same manner as in the method described in Example 7.

MS(ESI+): 595(M+H)

### Example 125

### (3R,4S)-N-{[2′,4′-dichloro-4-(trifluoromethoxy)biphenyl-3-yl]methyl}-3-phenylpiperidin-4-amine dihydrochloride

Using the compound obtained in Example 124, the title compound was obtained by the reaction and purification in the same manner as in the method described in Example 115.

MS(ESI+): 495(M−2HCl+H)

### Example 126

### 3-{2-[(3R,4S)-4-({[2′,4′-dichloro-4-(trifluoromethoxy)biphenyl-3-yl]methyl}amino)-3-phenylpiperidin-1-yl]-2-oxoethyl}-5,5-dimethyl-1,3-oxazolidine-2,4-dione monohydrochloride

Using the compound obtained in Example 125 and (5,5-dimethyl-2,4-dioxo-1,3-oxazolidin-3-yl)acetic acid, the reaction and purification were performed in the same manner as in the method described in Example 79. The obtained product was treated with 1 equivalent of hydrogen chloride/ethyl acetate to give the title compound. MS(ESI+): 664(M−HCl+H)

### Example 127

### 4-{[(3R,4S)-4-({[2′,4′-dichloro-4-(trifluoromethoxy)biphenyl-3-yl]methyl}amino)-3-phenylpiperidin-1-yl]carbonyl}piperidine-2,6-dione monohydrochloride

Using the compound obtained in Example 125 and 2,6-dioxopiperidine-4-carboxylic acid, the reaction and purification were performed in the same manner as in the method described in Example 79. The obtained product was treated with 1 equivalent of hydrogen chloride/ethyl acetate to give the title compound.

MS(ESI+): 634(M−HCl+H)

### Example 128

### (3R,4S)-1-[(1-acetylpiperidin-4-yl)carbonyl]-N-{[2′,4′-dichloro-4-(trifluoromethoxy)biphenyl-3-yl]methyl}-3-phenylpiperidin-4-amine monohydrochloride

Using the compound obtained in Example 125 and 1-acetylpiperidine-4-carboxylic acid, the reaction and purification were performed in the same manner as in the method described in Example 79. The obtained product was treated with 1 equivalent of hydrogen chloride/ethyl acetate to give the title compound.

MS(ESI+): 648(M−HCl+H)

### Example 129

### tert-butyl (3R,4S)-4-({[4′-chloro-2′-fluoro-4-(trifluoromethoxy)biphenyl-3-yl]methyl}amino)-3-phenylpiperidine-1-carboxylate

Using tert-butyl (3R,4S)-4-amino-3-phenylpiperidine-1-carboxylate and the compound obtained in Reference Example 29, the title compound was obtained by the reaction and purification in the same manner as in the method described in Example 7.

MS(ESI+): 579(M+H)

### Example 130

### (3R,4S)-N-{[4′-chloro-2′-fluoro-4-(trifluoromethoxy)biphenyl-3-yl]methyl}-3-phenylpiperidin-4-amine dihydrochloride

Using the compound obtained in Example 129, the title compound was obtained by the reaction and purification in the same manner as in the method described in Example 115.

MS(ESI+): 479(M−2HCl+H)

### Example 131

### 3-{2-[(3R,4S)-4-({[4′-chloro-2′-fluoro-4-(trifluoromethoxy)biphenyl-3-yl]methyl}amino)-3-phenylpiperidin-1-yl]-2-oxoethyl}-5,5-dimethyl-1,3-oxazolidine-2,4-dione monohydrochloride

Using the compound obtained in Example 130 and (5,5-dimethyl-2,4-dioxo-1,3-oxazolidin-3-yl)acetic acid, the reaction and purification were performed in the same manner as in the method described in Example 79. The obtained product was treated with 1 equivalent of hydrogen chloride/ethyl acetate to give the title compound.

MS(ESI+): 648(M−HCl+H)

### Example 132

### 4-{[(3R,4S)-4-({[4′-chloro-2′-fluoro-4-(trifluoromethoxy)biphenyl-3-yl]methyl}amino)-3-phenylpiperidin-1-yl]carbonyl}piperidine-2,6-dione monohydrochloride

To a solution of the compound (276 mg) obtained in Example 130, Et₃N (152 mg) and 2,6-dioxopiperidine-4-carboxylic acid (118 mg) in DMF (6 mL) were added WSC.HCl (144 mg) and HOBt.H₂O (115 mg), and the mixture was stirred at room temperature for 14 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (solvent gradient; 50→100% ethyl acetate/hexane). The obtained product was treated with 1 equivalent of hydrogen chloride/ethyl acetate to give the title compound.

Melting point: 162-164° C. Elemental analysis: C₃₁H₂₉Cl₂F₄N₃O_{4.}1.3H₂O Found C, 54.93; H, 4.85; N, 5.92; Calcd. C, 54.92; H, 4.70; N, 6.20. MS(ESI+): 618(M−HCl+H)

### Example 133

### 2-[(3R,4S)-4-({[4′-chloro-2′-fluoro-4-(trifluoromethoxy)biphenyl-3-yl]methyl}amino)-3-phenylpiperidin-1-yl]-2-oxoethanol

Using the compound obtained in Example 130 and glycolic acid, the title compound was obtained by the reaction and purification in the same manner as in the method described in Example 79.

MS(ESI+): 537(M+H)

### Example 134

(3R,4S)-1-[(1-acetylpiperidin-4-yl)carbonyl]-N-{[4′-chloro-2′-fluoro-4-(trifluoromethoxy)biphenyl-3-yl]methyl}-3-phenylpiperidin-4-amine monohydrochloride

Using the compound obtained in Example 130 and 1-acetylpiperidine-4-carboxylic acid, the reaction and purification were performed in the same manner as in the method described in Example 79. The obtained product was treated with 1 equivalent of hydrogen chloride/ethyl acetate to give the title compound.

MS(ESI+): 632(M−HCl+H)

### Example 135

### tert-butyl (3R,4S)-4-({[4′-cyano-4-(trifluoromethoxy)biphenyl-3-yl]methyl}amino)-3-phenylpiperidine-1-carboxylate

Using tert-butyl (3R,4S)-4-amino-3-phenylpiperidine-1-carboxylate and the compound obtained in Reference Example 8, the title compound was obtained by the reaction and purification in the same manner as in the method described in Example 7.

MS(ESI+): 552(M+H)

### Example 136

### 3′-({[(3R,4S)-3-phenylpiperidin-4-yl]amino}methyl)-4′-(trifluoromethoxy)biphenyl-4-carbonitrile dihydrochloride

Using the compound obtained in Example 135, the title compound was obtained by the reaction and purification in the same manner as in the method described in Example 115.

MS(ESI+): 452(M−2HCl+H)

### Example 137

### 3′-[({(3R,4S)-1-[(5,5-dimethyl-2,4-dioxo-1,3-oxazolidin-3-yl)acetyl]-3-phenylpiperidin-4-yl}amino)methyl]-4′-(trifluoromethoxy)biphenyl-4-carbonitrile monohydrochloride

To a solution of the compound (262 mg) obtained in Example 136, Et₃N (152 mg) and (5,5-dimethyl-2,4-dioxo-1,3-oxazolidin-3-yl)acetic acid (140 mg) in DMF (6 mL) were added WSC.HCl (144 mg) and HOBt.H₂O (115 mg), and the mixture was stirred at room temperature for 14 hr. The reaction mixture was poured into water, and the product was extracted with ethyl acetate. The organic layer was washed with a saturated aqueous sodium hydrogen carbonate solution and brine and dried, and the solvent was evaporated under reduced pressure. The obtained residue was purified by silica gel column chromatography (solvent gradient; 50→100% ethyl acetate/hexane). The obtained product was treated with 1 equivalent of hydrogen chloride/ethyl acetate to give the title compound.

Melting point: 197-199° C. Elemental analysis: C₃₃H₃₂ClF₃N₄O₅.0.5H₂O Found C, 59.78; H, 4.85; N, 8.42; Calcd. C, 59.50; H, 4.99; N, 8.41. MS(ESI+): 620(M−HCl+H)

The compounds described in Examples 47-137 are as follows (Tables 8-14).

TABLE 8

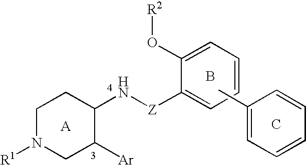

| Ex. No. | R¹ | 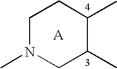 | Ar | Z | 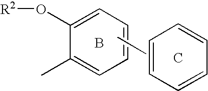 | Addi- tives | MS (ESI) |
|---|---|---|---|---|---|---|---|
| 47 | 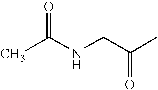 | 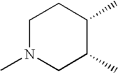 (3R, 4S) | 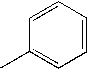 | CH₂ | 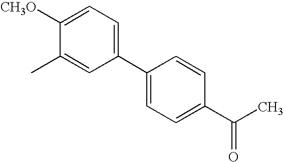 | HCl | 514 (M-HCl + H)⁺ |
| 48 | 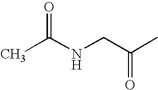 | 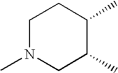 (3R, 4S) | 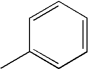 | CH₂ | 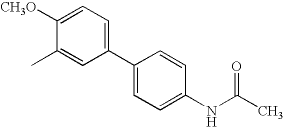 | HCl | 529 (M-HCl + H)⁺ |
| 49 | 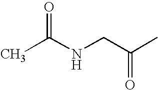 | 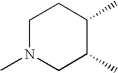 (3R, 4S) | 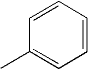 | CH₂ | 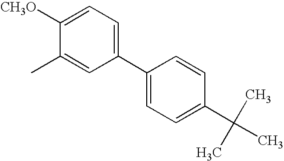 | HCl | 528 (M-HCl + H)⁺ |
| 50 | 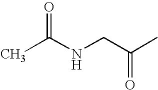 | 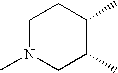 (3R, 4S) | 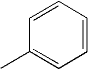 | CH₂ | 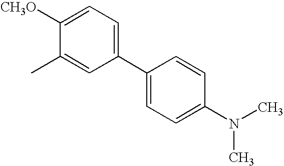 | 2HCl | 515 (M-2HCl + H)⁺ |
| 51 | 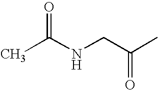 | 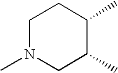 (3R, 4S) | 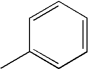 | CH₂ | 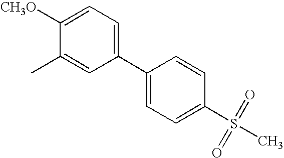 | | 550 (M + H)⁺ |
| 52 | 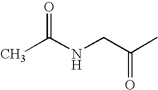 | 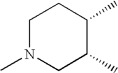 (3R, 4S) | 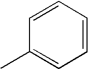 | CH₂ | 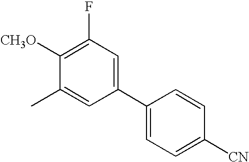 | | 515 (M + H)⁺ |
| 53 | 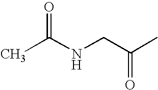 | 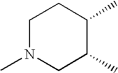 (3R, 4S) | 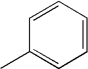 | CH₂ | 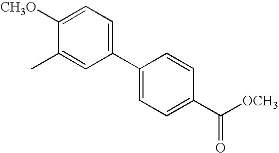 | | 530 (M + H)⁺ |
| 54 | 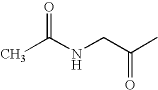 | 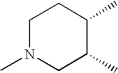 (3R, 4S) | 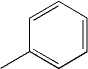 | CH₂ | 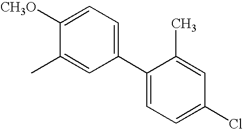 | | 520 (M + H)⁺ |
| 55 | 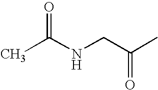 | 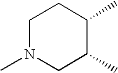 (3R, 4S) | 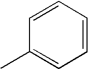 | CH₂ | 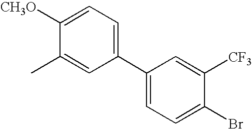 | | 618, 620 (M + H)⁺ |
| 56 | 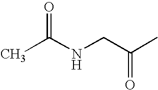 | 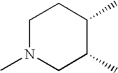 (3R, 4S) | 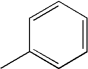 | CH₂ | 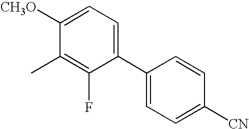 | | 515 (M + H)⁺ |
| 57 | 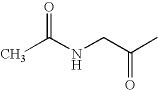 | 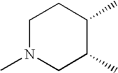 (3R, 4S) | 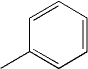 | CH₂ | 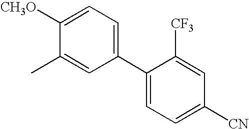 | | 565 (M + H)⁺ |
| 58 | 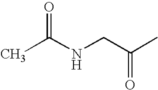 | 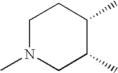 (3R, 4S) | 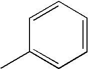 | CH₂ | 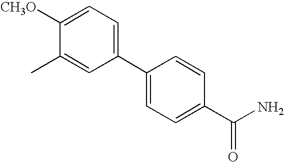 | | 515 (M + H)⁺ |
| 59 | 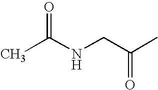 | 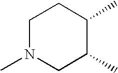 (3R, 4S) | 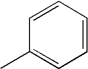 | CH₂ | 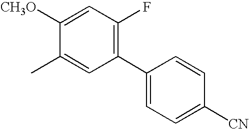 | | 515 (M + H)⁺ |

TABLE 9

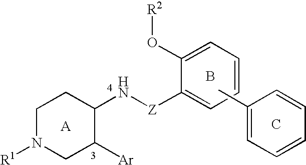

| Ex. No. | R¹ | 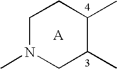 | Ar | Z | 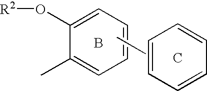 | Addi- tives | MS (ESI) |
|---|---|---|---|---|---|---|---|
| 60 | 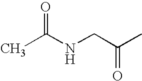 | 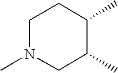 (3R, 4S) | 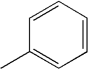 | CH₂ | 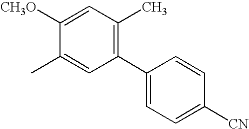 | | 511 (M + H)⁺ |
| 61 | 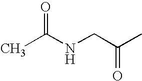 | 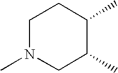 (3R, 4S) | 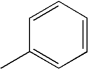 | CH₂ | 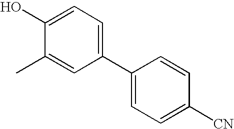 | | 483 (M + H)⁺ |
| 62 | 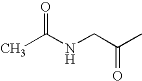 | 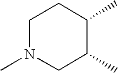 (3R, 4S) | 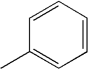 | CH₂ | 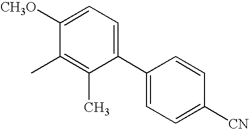 | | 511 (M + H)⁺ |
| 63 | 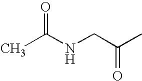 | 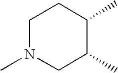 (3R, 4S) | 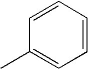 | C(CH₃)H (1:1 diastereo- mixture) | 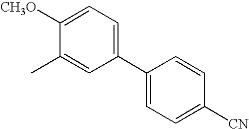 | | 511 (M + H)⁺ |
| 64 | 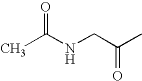 | 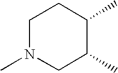 (3R, 4S) | 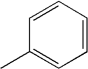 | C(CH₃)H (shorter Rt) | 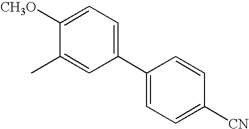 | | 511 (M + H)⁺ |
| 65 | 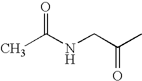 | 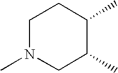 (3R, 4S) | 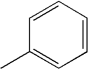 | C(CH₃)H (longer Rt) | 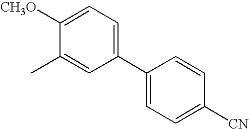 | | 511 (M + H)⁺ |
| 66 | 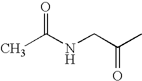 | 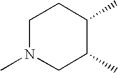 (3R, 4S) | 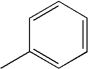 | CH₂ | 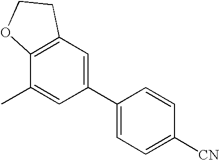 | | 509 (M + H)⁺ |
| 67 | 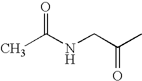 | 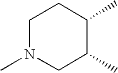 (3R, 4S) | 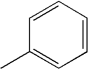 | CH₂ | 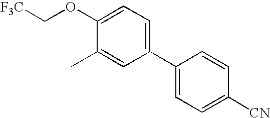 | HCl | 565 (M-HCl + H)⁺ |
| 68 | 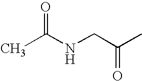 | 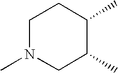 (3R, 4S) | 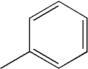 | CH₂ | 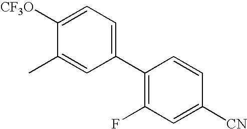 | | 569 (M + H)⁺ |
| 69 | 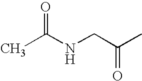 | 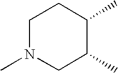 (3R, 4S) | 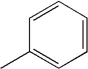 | CH₂ | 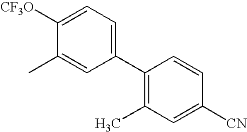 | | 565 (M + H)⁺ |
| 70 | 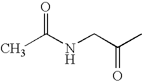 | 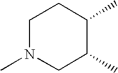 (3R, 4S) | 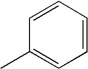 | CH₂ | 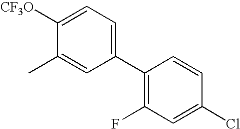 | | 578 (M + H)⁺ |
| 71 | 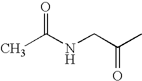 | 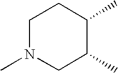 (3R, 4S) | 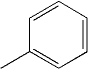 | CH₂ | 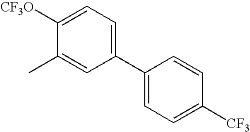 | | 594 (M + H)⁺ |
| 72 | 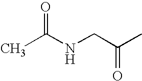 | 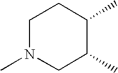 (3R, 4S) | 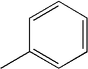 | CH₂ | 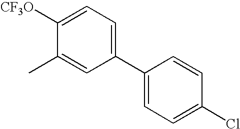 | | 560 (M + H)⁺ |

TABLE 10

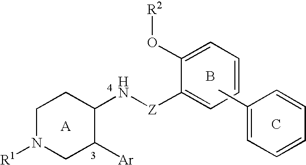

| Ex. No. | R¹ | 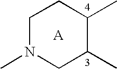 | Ar | Z | 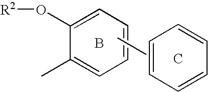 | Addi- tives | MS (ESI) |
|---|---|---|---|---|---|---|---|
| 73 | 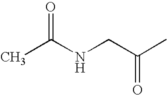 | 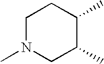 (3R, 4S) | 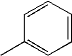 | CH₂ | 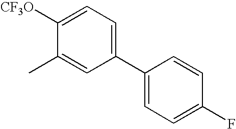 | | 544 (M + H)⁺ |
| 74 | 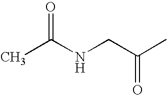 | 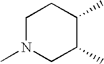 (3R, 4S) | 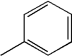 | CH₂ | 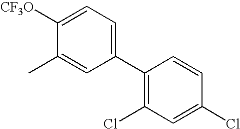 | | 594 (M + H)⁺ |
| 75 | 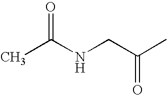 | 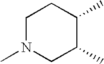 (3R, 4S) | 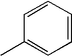 | CH₂ | 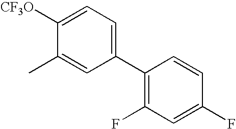 | | 562 (M + H)⁺ |
| 76 | 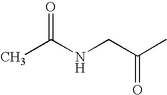 | 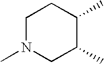 (3R, 4S) | 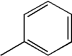 | CH₂ | 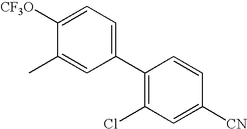 | | 585 (M + H)⁺ |
| 77 | 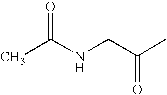 | 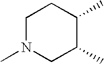 (3R, 4S) | 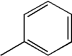 | CH₂ | 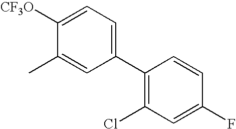 | | 578 (M + H)⁺ |
| 78 | 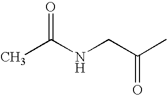 | 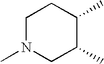 (3R, 4S) | 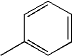 | CH₂ | 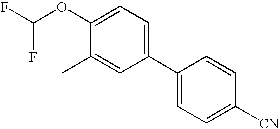 | | 533 (M + H)⁺ |
| 79 | 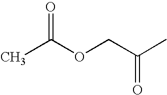 | 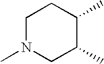 (3R, 4S) | 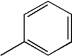 | CH₂ | 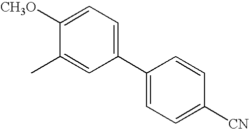 | | 498 (M + H)⁺ |
| 80 | 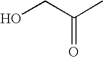 | 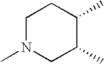 (3R, 4S) | 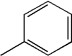 | CH₂ | 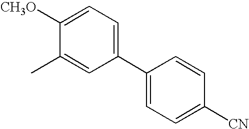 | | 456 (M + H)⁺ |
| 81 | 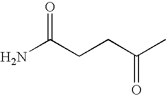 | 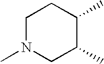 (3R, 4S) | 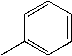 | CH₂ | 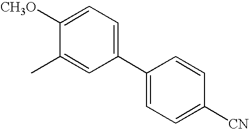 | | 497 (M + H)⁺ |
| 82 | 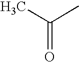 | 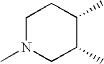 (3R, 4S) | 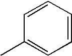 | CH₂ | 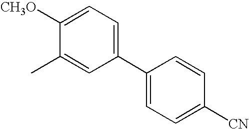 | HCl | 440 (M-HCl + H)⁺ |
| 83 | 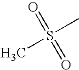 | 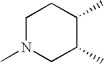 (3R, 4S) | 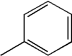 | CH₂ | 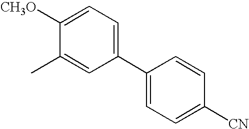 | | 476 (M + H)⁺ |
| 84 | 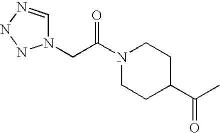 | 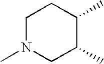 (3R, 4S) | 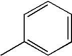 | CH₂ | 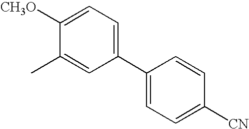 | HCl | 619 (M-HCl + H)⁺ |
| 85 | 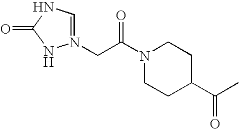 | 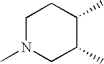 (3R, 4S) | 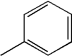 | CH₂ | 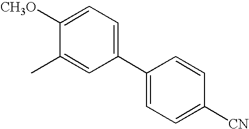 | HCl | 634 (M-HCl + H)⁺ |

TABLE 11

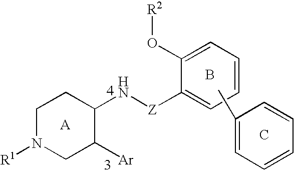

| Ex. No. | RHU 1 | 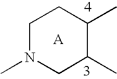 | Ar |
|---|---|---|---|
| 86 | 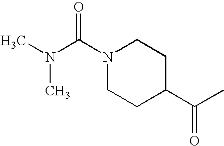 | 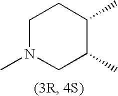 | 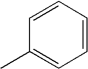 |
| 87 | 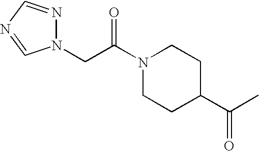 | 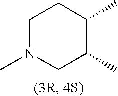 | 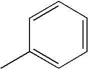 |
| 88 | 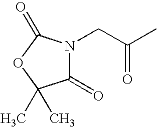 | 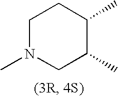 | 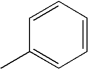 |
| 89 | 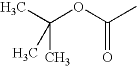 | 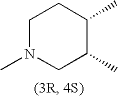 | 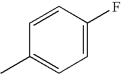 |
| 90 | H | 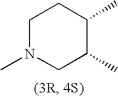 | 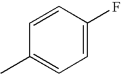 |
| 91 | 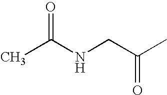 | 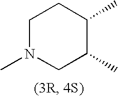 | 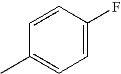 |
| 92 | 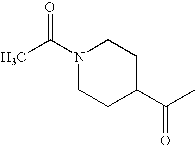 | 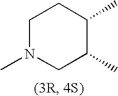 | 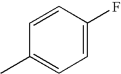 |
| 93 | 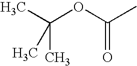 | 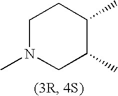 | 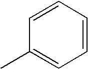 |
| 94 | H | 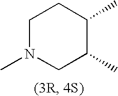 | 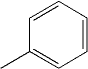 |
| 95 | H | 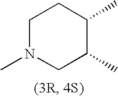 | 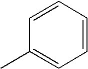 |
| 96 | 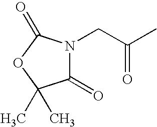 | 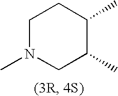 | 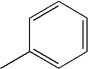 |
| 97 | 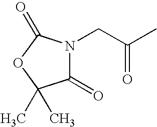 | 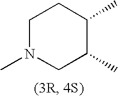 | 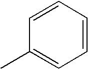 |
| 98 | 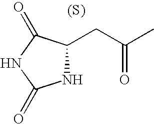 | 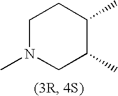 | 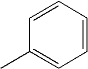 |

| Ex. No. | Z | 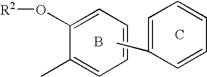 | Additives | MS (ESI) |
|---|---|---|---|---|
| 86 | CH₂ | 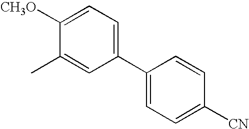 | | 580 (M + H)⁺ |
| 87 | CH₂ | 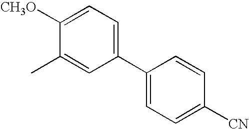 | HCl | 618 (M − HCl + H)⁺ |
| 88 | CH₂ | 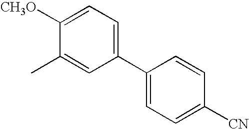 | | 567 (M + H)⁺ |
| 89 | CH₂ | 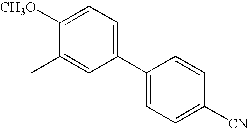 | | 516 (M + H)⁺ |
| 90 | CH₂ | 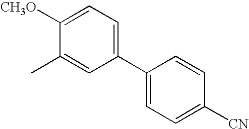 | 2HCl | 416 (M − 2HCl + H)⁺ |
| 91 | CH₂ | 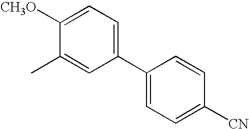 | | 515 (M + H)⁺ |
| 92 | CH₂ | 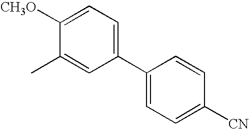 | | 569 (M + H)⁺ |
| 93 | CH₂ | 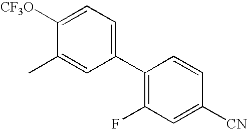 | | 570 (M + H)⁺ |
| 94 | CH₂ | 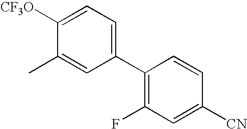 | 2HCl | 470 (M − 2HCl + H)⁺ |
| 95 | CH₂ | 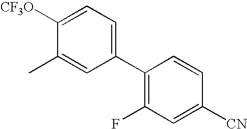 | 2TFA | 470 (M − 2TFA + H)⁺ |
| 96 | CH₂ | 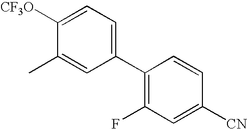 | | 639 (M + H)⁺ |
| 97 | CH₂ | 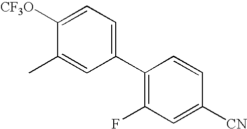 | HCl | 639 (M − HCl + H)⁺ |
| 98 | CH₂ | 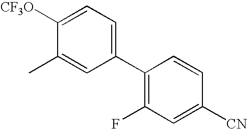 | | 610 (M + H)⁺ |

TABLE 12

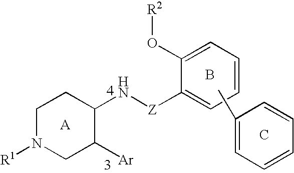

| Ex. No. | R¹ | 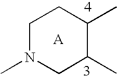 | Ar | Z | 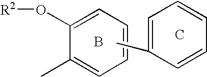 | Additives | MS (ESI) |
|---|---|---|---|---|---|---|---|
| 99 | 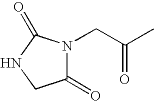 | 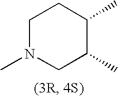 | 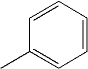 | CH₂ | 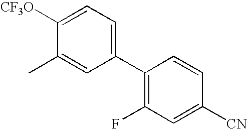 | | 610 (M + H)⁺ |
| 100 | 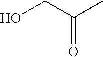 | 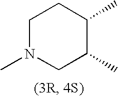 | 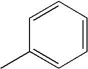 | CH₂ | 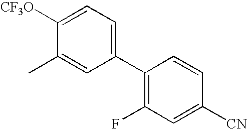 | | 528 (M + H)⁺ |
| 101 | 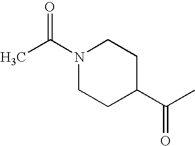 | 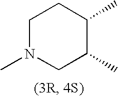 | 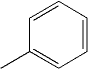 | CH₂ | 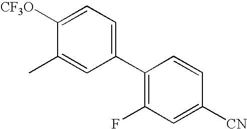 | | 623 (M + H)⁺ |
| 102 | 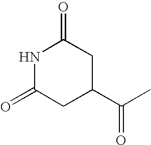 | 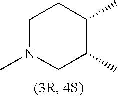 | 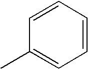 | CH₂ | 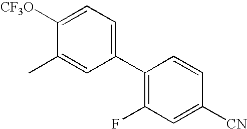 | | 609 (M + H)⁺ |
| 103 | 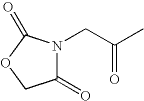 | 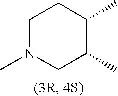 | 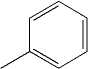 | CH₂ | 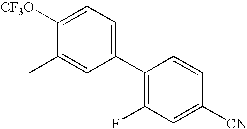 | | 611 (M + H)⁺ |
| 104 | 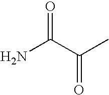 | 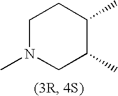 | 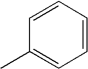 | CH₂ | 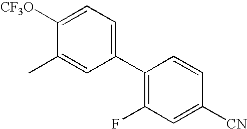 | | 541 (M + H)⁺ |
| 105 | 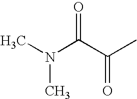 | 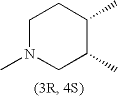 | 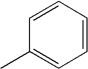 | CH₂ | 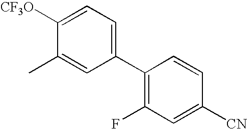 | | 569 (M + H)⁺ |
| 106 | 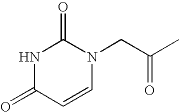 | 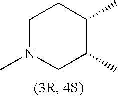 | 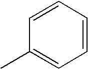 | CH₂ | 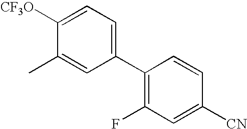 | | 622 (M + H)⁺ |
| 107 | 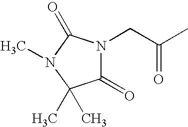 | 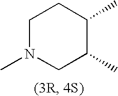 | 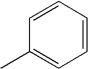 | CH₂ | 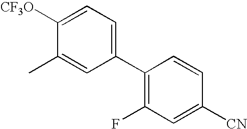 | HCl | 652 (M −HCl +H)⁺ |
| 108 | 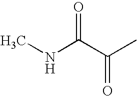 | 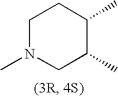 | 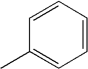 | CH₂ | 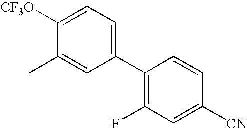 | HCl | 555 (M −HCl +H)⁺ |
| 109 | 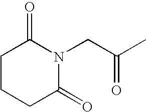 | 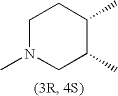 | 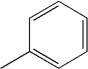 | CH₂ | 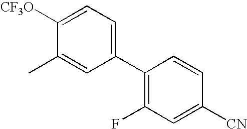 | HCl | 623 (M −HCl +H)⁺ |
| 110 | 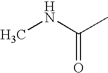 | 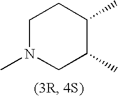 | 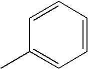 | CH₂ | 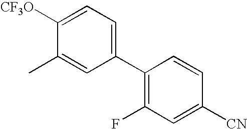 | | 527 (M + H)⁺ |
| 111 | 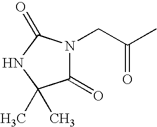 | 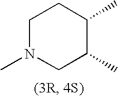 | 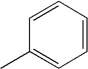 | CH₂ | 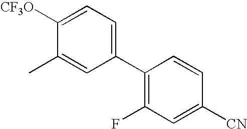 | HCl | 638 (M −HCl +H)⁺ |

TABLE 13

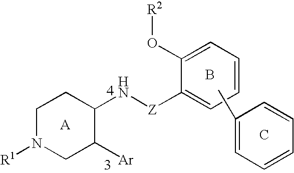

| Ex. No. | R¹ | 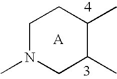 | Ar | Z | 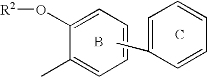 | Additives | MS (ESI) |
|---|---|---|---|---|---|---|---|
| 112 | 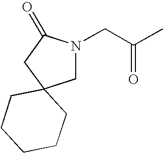 | 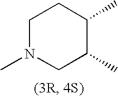 | 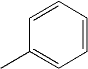 | CH₂ | 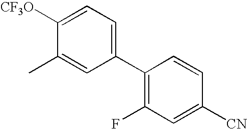 | HCl | 653 (M −HCl +H)⁺ |
| 113 | 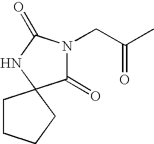 | 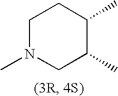 | 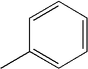 | CH₂ | 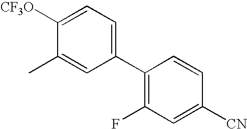 | HCl | 664 (M −HCl +H)⁺ |
| 114 | 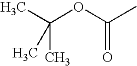 | 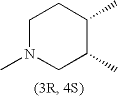 | 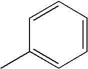 | CH₂ | 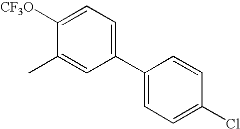 | | 561 (M + H)⁺ |
| 115 | H | 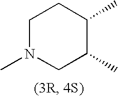 | 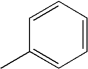 | CH₂ | 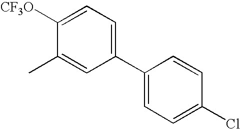 | 2HCl | 461 (M −2HCl +H)⁺ |
| 116 | 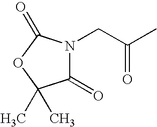 | 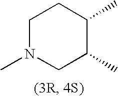 | 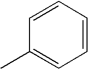 | CH₂ | 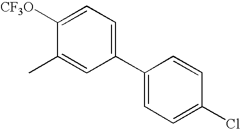 | HCl | 630 (M −HCl +H)⁺ |
| 117 | 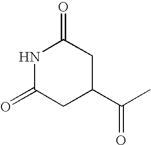 | 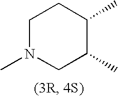 | 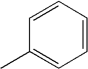 | CH₂ | 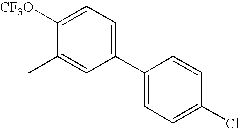 | HCl | 600 (M −HCl +H)⁺ |
| 118 | 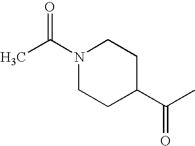 | 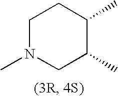 | 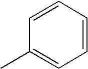 | CH₂ | 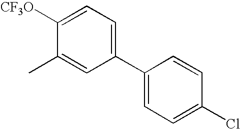 | HCl | 614 (M −HCl +H)⁺ |
| 119 | 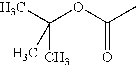 | 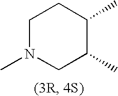 | 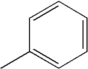 | CH₂ | 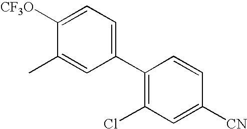 | | 586 (M + H)⁺ |
| 120 | H | 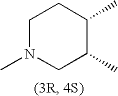 | 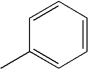 | CH₂ | 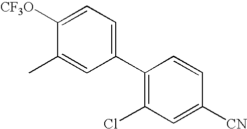 | 2HCl | 486 (M −2HCl +H)⁺ |
| 121 | 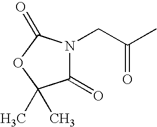 | 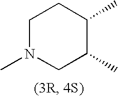 | 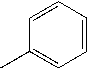 | CH₂ | 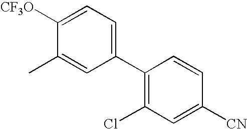 | HCl | 655 (M −HCl +H)⁺ |
| 122 | 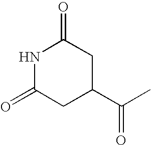 | 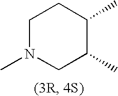 | 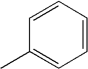 | CH₂ | 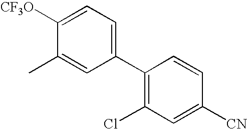 | HCl | 625 (M −HCl +H)⁺ |
| 123 | 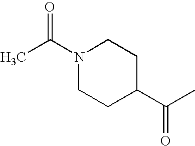 | 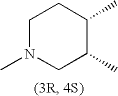 | 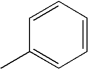 | CH₂ | 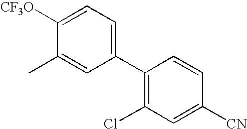 | HCl | 639 (M −HCl +H)⁺ |
| 124 | 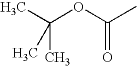 | 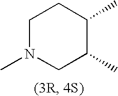 | 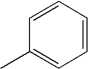 | CH₂ | 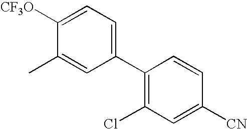 | | 595 (M + H)⁺ |

TABLE 14

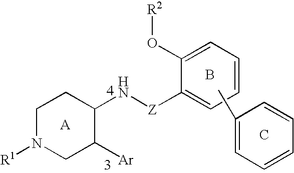

| Ex. No. | R¹ | 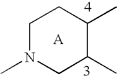 | Ar | Z | 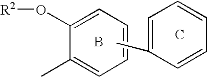 | Additives | MS (ESI) |
|---|---|---|---|---|---|---|---|
| 125 | H | 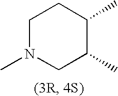 | 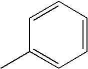 | CH₂ | 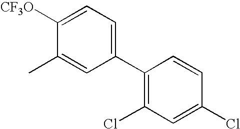 | 2HCl | 495 (M −2HCl +H)⁺ |
| 126 | 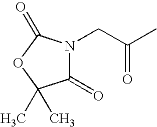 | 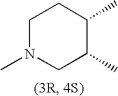 | 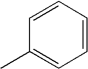 | CH₂ | 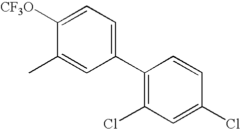 | HCl | 664 (M −HCl +H)⁺ |
| 127 | 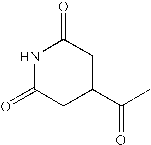 | 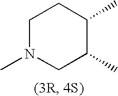 | 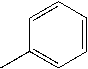 | CH₂ | 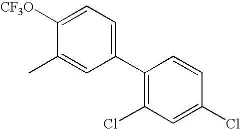 | HCl | 634 (M −HCl +H)⁺ |
| 128 | 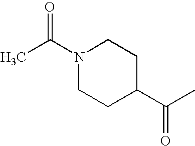 | 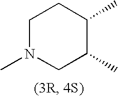 | 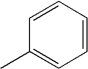 | CH₂ | 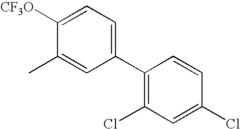 | HCl | 648 (M −HCl +H)⁺ |
| 129 | 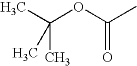 | 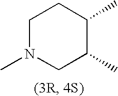 | 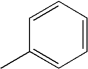 | CH₂ | 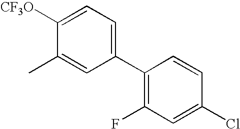 | | 579 (M + H)⁺ |
| 130 | H | 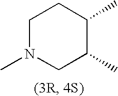 | 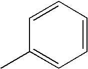 | CH₂ | 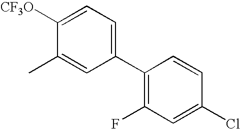 | 2HCl | 479 (M −2HCl +H)⁺ |
| 131 | 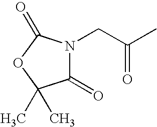 | 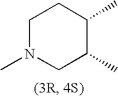 | 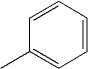 | CH₂ | 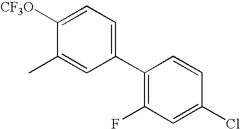 | HCl | 648 (M −HCl +H)⁺ |
| 132 | 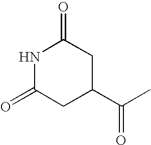 | 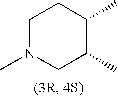 | 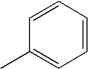 | CH₂ | 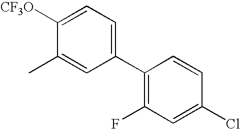 | HCl | 618 (M −HCl +H)⁺ |
| 133 | 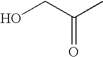 | 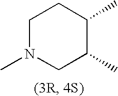 | 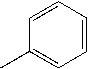 | CH₂ | 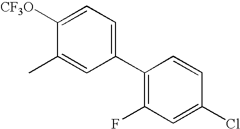 | | 537 (M + H)⁺ |
| 134 | 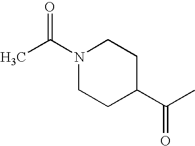 | 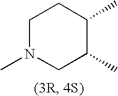 | 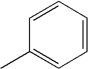 | CH₂ | 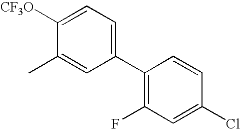 | HCl | 632 (M −HCl +H)⁺ |
| 135 | 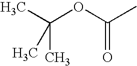 | 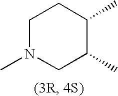 | 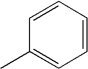 | CH₂ | 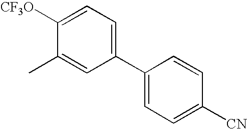 | | 552 (M + H)⁺ |
| 136 | H | 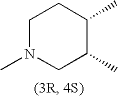 | 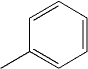 | CH₂ | 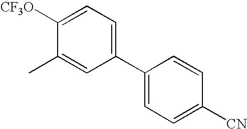 | 2HCl | 452 (M −2HCl +H)⁺ |
| 137 | 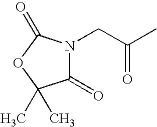 | 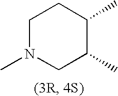 | 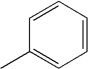 | CH₂ | 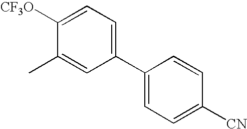 | HCl | 620 (M −HCl +H)⁺ |

### Example 138

### 3′-[({(3R,4S)-1-[(2,6-Dioxopiperidin-1-yl)acetyl]-3-phenylpiperidin-4-yl}amino)methyl]-2-fluoro-4′-(trifluoromethoxy)biphenyl-4-carbonitrile monomaleate

To the compound (0.35 g) obtained in Example 109 was added a saturated aqueous sodium hydrogen carbonate solution. The resulting mixture was extracted with ethyl acetate. The extract was dried over anhydrous magnesium sulfate and concentrated under reduced pressure to give 3′-[({(3R,4S)-1-[(2,6-dioxopiperidin-1-yl)acetyl]-3-phenylpiperidin-4-yl}amino)methyl]-2-fluoro-4′-(trifluoromethoxy)biphenyl-4-carbonitrile as a colorless solid. The obtained colorless solid (0.05 g) was dissolved in ethanol (2 mL) and acetone (4 mL) and then maleic acid (0.0095 g) was added thereto. The resulting mixture was stirred at room temperature for 14 hr. A mixture of hexane and diisopropyl ether was added thereto and the precipitated solid was collected by filtration to give the title compound as white powder (0.052 g).

MS(ESI+): 623 (M−116+H) Elemental analysis: C₃₇H₃₄N₄O₈F₄ Found C, 59.77; H, 4.62; N, 7.40. Calcd. C, 60.16; H, 4.64; N, 7.58.

### Example 139

### 3′-[({(3R,4S)-1-[(2,6-Dioxopiperidin-1-yl)acetyl]-3-phenylpiperidin-4-yl}amino)methyl]-2-fluoro-4′-(trifluoromethoxy)biphenyl-4-carbonitrile monofumarate

The colorless solid of 3′-[({(3R,4S)-1-[(2,6-dioxopiperidin-1-yl)acetyl]-3-phenylpiperidin-4-yl}amino)methyl]-2-fluoro-4′-(trifluoromethoxy)biphenyl-4-carbonitrile (0.05 g) was dissolved in ethanol (2 mL) and acetone (3 mL) and then fumaric acid (0.0095 g) was added thereto. The resulting mixture was stirred at room temperature for 14 hr. A mixture of hexane and diisopropyl ether was added thereto and the precipitated solid was collected by filtration to give the title compound as white powder (0.052 g).

MS(ESI+): 623 (M−116+H) Elemental analysis: C₃₇H₃₄N₄O₈F₄ Found C, 59.74; H, 4.57; N, 7.26. Calcd. C, 60.16; H, 4.64; N, 7.58.

### Example 140

### 4-{[(3R,4S)-4-({[4′-Chloro-4-(trifluoromethoxy)biphenyl-3-yl]methyl}amino)-3-phenylpiperidin-1-yl]carbonyl}piperidine-2,6-dione monohydrate

A mixture of (3R,4S)-N-{[4′-chloro-4-(trifluoromethoxy)biphenyl-3-yl]methyl}-3-phenylpiperidin-4-amine dihydrochloride (9.8 g), 2,6-dioxopiperidine-4-carboxylic acid (5.0 g), WSC.HCL (6.1 g), HOBt.H₂O (4.9 g), triethylamine (6.4 g) and DMF (200 mL) was stirred at room temperature for 14 hr. The reaction mixture was poured into water, and the product was extracted with ethyl acetate. The organic layer was washed with a saturated aqueous sodium hydrogen carbonate solution and brine and dried over magnesium sulfate, and the solvent was evaporated under reduced pressure. The obtained residue was purified by silica gel column chromatography (solvent gradient; 20→50% ethyl acetate/hexane)to give 4-{[(3R,4S)-4-({[4′-chloro-4-(trifluoromethoxy)biphenyl-3-yl]methyl}amino)-3-phenylpiperidin-1-yl]carbonyl}piperidine-2,6-dione (11.5 g) as an amorphous solid. The obtained amorphous solid (8 g) was dissolved in ethanol (60 mL) and then water (15 mL) was added thereto. The resulting mixture was stirred at room temperature for 14 hr to lead crystallization. The crystals were collected by filtration, washed with water and dried to give the title compound as white powdery crystals (7.3 g, Yield 93%).

Elemental analysis: C₃₁H₂₉N₃O₄ClF₃.H₂O Found C, 60.02; H, 4.96; N, 6.72. Calcd. C, 60.24; H, 5.06; N, 6.80.

### Example 141

### 4-{[(3R,4S)-4-({[4′-Chloro-4-(trifluoromethoxy)biphenyl-3-yl]methyl}amino)-3-phenylpiperidin-1-yl]carbonyl}piperidine-2,6-dione monomaleate

The compound (0.30 g) obtained in Example 140 was dissolved in toluene (1.5 mL). To the solution was added a solution of maleic acid (0.06 g) in 1,4-dioxane. The resulting mixture was stirred at room temperature and then concentrated under reduced pressure. The residue was treated with toluene to give the crude solid (0.36 g). The obtained solid (0.28 g) was recrystallized from ethanol/acetone to give the title compound (0.12 g, Yield 45%) as white powder.

Elemental analysis: C₃₅H₃₃N₃O₈ClF₃ Found C, 58.48; H, 4.51; N, 5.83. Calcd. C, 58.70; H, 4.64; N, 5.87.

### Example 142

### 4-{[(3R,4S)-4-({[4′-Chloro-4-(trifluoromethoxy)biphenyl-3-yl]methyl}amino)-3-phenylpiperidin-1-yl]carbonyl}piperidine-2,6-dione monofumarate

The compound (0.30 g) obtained in Example 140 was dissolved in toluene (1.5 mL). To the solution was added a solution′of fumaric acid (0.06 g) in 1,4-dioxane. The resulting mixture was stirred at room temperature and then concentrated under reduced pressure. The residue was treated with toluene to give the crude solid (0.30 g). The obtained solid (0.28 g) was recrystallized from ethanol/acetone to give the title compound (0.16 g, Yield 57%) as white powder.

Elemental analysis: C₃₅H₃₃N₃O₈ClF₃ Found C, 58.47; H, 4.56; N, 5.86. Calcd. C, 58.70; H, 4.64; N, 5.87.

### Example 143

### 2-Fluoro-3′-({[(3R,4S)-1-(3-methyl-1,2,4-thiadiazol-5-yl)-3-phenylpiperidin-4-yl]amino}methyl)-4′-(trifluoromethoxy)biphenyl-4-carbonitrile monohydrochloride

To a stirred mixture of acetamidine hydrochloride (21 g) and perchloromethyl mercaptan (37 g) in dichloromethane (180 mL) at −10° C. was added an aqueous sodium hydroxide solution (44 g in 67 mL) dropwise over the period of 1 hr. The resulting mixture was stirred at room temperature for 2 hr and then filtered. The filtrate was washed with water, dried over anhydrous magnesium sulfate and distilled (47° C./10 mmHg) to give 5-chloro-3-methyl-1,2,4-thiadiazole (12 g) as colorless oil.

To a mixture of 2-fluoro-3′-({[(3R,4S)-3-phenylpiperidin-4-yl]amino}methyl)-4′-(trifluoromethoxy)biphenyl-4-carbonitrile dihydrochloride (0.27 g) and triethylamine (0.15 g) in ethanol (5 mL) was added 5-chloro-3-methyl-1,2,4-thiadiazole (0.081 g) at 0° C. The resulting mixture was stirred at room temperature for 1.5 hr. The reaction mixture was concentrated under reduced pressure and the residue was partitioned between water and ethyl acetate. The organic layer was washed sequentially with a saturated aqueous sodium hydrogen carbonate solution and brine, dried over anhydrous magnesium sulfate and concentrated under reduced pressure. The residue was purified by silica gel column chromatography (ethyl acetate/hexane) to give 2-fluoro-3′-({[(3R,4S)-1-(3-methyl-1,2,4-thiadiazol-5-yl)-3-phenylpiperidin-4-yl]amino}methyl)-4′-(trifluoromethoxy)-biphenyl-4-carbonitrile. The obtained compound was treated with 4N hydrogen chloride/ethyl acetate solution and then concentrated under reduced pressure. The residue was crystallized from ethanol/diisopropyl ether to give the title compound (0.23 g, Yield 75%) as white powder.

Elemental analysis: C₂₉H₂₆N₅OSClF₄.0.5H₂O Found C, 56.41; H, 4.50; N, 11.38. Calcd. C, 56.81; H, 4.44; N, 11.42.

### Example 144

### Ethyl [(3R,4S)-4-({[4′-cyano-2′-fluoro-4-(trifluoromethoxy)biphenyl-3-yl]methyl}amino)-3-phenylpiperidin-1-yl](oxo)acetate

To a mixture of 2-fluoro-3′-({[(3R,4S)-3-phenylpiperidin-4-yl]amino}methyl)-4′-(trifluoromethoxy)biphenyl-4-carbonitrile dihydrochloride (2.0 g) and triethylamine (0.75 g) in THF (50 mL) was added ethyl chlorooxoacetate (0.50 g) at 0° C. The resulting mixture was stirred at room temperature for 2 hr. The reaction mixture was concentrated under reduced pressure and the residue was partitioned between water and ethyl acetate. The organic layer was washed sequentially with a saturated aqueous sodium hydrogen carbonate solution and brine, dried over anhydrous magnesium sulfate and concentrated under reduced pressure. The residue was purified by silica gel column chromatography (ethyl acetate/hexane) to give the title compound (1.3 g, Yield 61%) as an amorphous solid.

Elemental analysis: C₃₀H₂₇N₃O₄F₄ Found C, 63.06; H, 4.78; N, 7.35. Calcd. C, 63.26; H, 4.78; N, 7.38.

### Example 145

### 3′-({[(3R,4S)-1-(3,3-Dimethyl-2-oxobutanoyl)-3-phenylpiperidin-4-yl]amino}methyl)-2-fluoro-4′-(trifluoromethoxy)biphenyl-4-carbonitrile monohydrochloride

To a mixture of 2-fluoro-3′-({[(3R,4S)-3-phenylpiperidin-4-yl]amino}methyl)-4′-(trifluoromethoxy)biphenyl-4-carbonitrile dihydrochloride (0.27 g), 3,3-dimethyl-2-oxobutyric acid sodium salt (0.10 g) and triethylamine (0.15 g) in DMF (6 mL) was added HOBt.H₂O (0.12 g) and WSC.HCl (0.14 g). The resulting mixture was stirred at room temperature for 24 hr. The reaction mixture was partitioned between water and ethyl acetate. The organic layer was washed sequentially with saturated aqueous sodium hydrogen carbonate solution and brine, dried over anhydrous magnesium sulfate and concentrated under reduced pressure. The residue was purified by silica gel column chromatography (ethyl acetate/hexane) to give 3′-({[(3R,4S)-1-(3,3-dimethyl-2-oxobutanoyl)-3-phenylpiperidin-4-yl]amino}methyl)-2-fluoro-4′-(trifluoromethoxy)biphenyl-4-carbonitrile. The obtained compound was treated with 4N hydrogen chloride/ethyl acetate solution and then concentrated under reduced pressure. The residue was crystallized from diisopropyl ether/n-hexane/ethyl acetate to give the title compound (0.13 g, Yield 41%) as an amorphous solid.

Elemental analysis: C₃₂H₃₂N₃O₃ClF₄ Found C, 61.89; H, 5.55; N, 6.46. Calcd. C, 62.19; H, 5.22; N, 6.80.

### Example 146

### tert-Butyl (3R,4S)-4-({[4′-cyano-2′-fluoro-4-(trifluoromethoxy)biphenyl-3-yl]methyl}amino)-3-phenyl-1,4′-bipiperidine-1′-carboxylate

To a mixture of 2-fluoro-3′-({[(3R,4S)-3-phenylpiperidin-4-yl]amino}methyl)-4′-(trifluoromethoxy)biphenyl-4-carbonitrile dihydrochloride (0.11 g), 1-Boc-piperidone (4.0 g) and acetic acid (1 mL) in DMF/THF (1:10, 11 mL) was added NaBH(OAc)₃ (2.1 g). The resulting mixture was stirred at room temperature for 16 hr. The reaction mixture was partitioned between water and ethyl acetate. The organic layer was washed sequentially with saturated aqueous sodium hydrogen carbonate solution and brine, dried over anhydrous magnesium sulfate and concentrated under reduced pressure. The residue was purified by silica gel column chromatography (ethyl acetate/hexane) to give the title compound (1.1 g, Yield 80%) as an amorphous solid.

MS(ESI+): 653 (M+H)

### Example 147

### 3′-({[(3R,4S)-1′-Acetyl-3-phenyl-1,4′-bipiperidin-4-yl]amino}methyl)-2-fluoro-4′-(trifluoromethoxy)biphenyl-4-carbonitrile

A mixture of the compound (0.88 g) obtained in Example 146 and 4N hydrogen chloride/ethyl acetate solution (1.5 mL) was stirred at 60° C. for 1.5 hr. The reaction mixture was concentrated under reduced pressure to give 2-fluoro-3′-({[(3R,4S)-3-phenyl-1,4′-bipiperidin-4-yl]amino}methyl)-4′-(trifluoromethoxy)biphenyl-4-carbonitrile dihydrochloride (0.82 g) as an amorphous solid.

To a mixture of the obtained amorphous solid (0.33 g) and triethylamine (0.20 g) in THF (7 mL) was added acetyl chloride (0.047 g) at 0° C. The resulting mixture was stirred at that temperature for 2 hr. The reaction mixture was partitioned between water and ethyl acetate. The organic layer was washed sequentially with a saturated aqueous sodium hydrogen carbonate solution and brine, dried over anhydrous magnesium sulfate and concentrated under reduced pressure. The residue was purified by silica gel column chromatography (ethyl acetate/hexane) to give the title compound (0.23 g, Yield 77%) as an amorphous solid.

MS(ESI+): 595 (M+H) Elemental analysis: C₃₃H₃₄N₃O₂F₄.0.5H₂O Found C, 65.58; H, 5.84; N, 8.93. Calcd. C, 65.66; H, 5.84; N, 9.28.

The compounds described in Examples 138-147 are as follows (Table 15).

TABLE 15

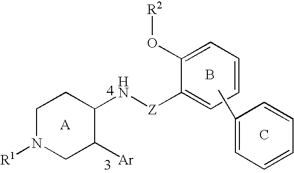

| Ex. No. | R¹ | 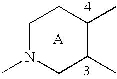 | Ar | Z |
|---|---|---|---|---|
| 138 | 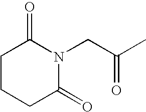 | 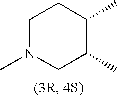 | 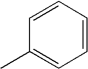 | CH₂ |
| 139 | 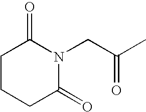 | 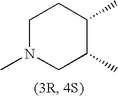 | 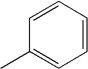 | CH₂ |
| 140 | 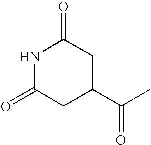 | 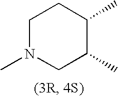 | 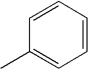 | CH₂ |
| 141 | 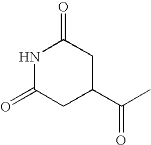 | 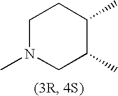 | 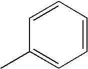 | CH₂ |
| 142 | 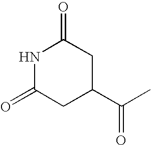 | 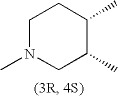 | 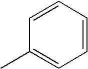 | CH₂ |
| 143 | 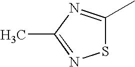 | 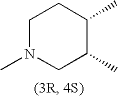 | 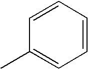 | CH₂ |
| 144 | 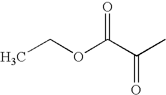 | 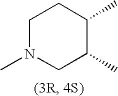 | 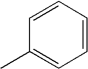 | CH₂ |
| 145 | 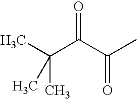 | 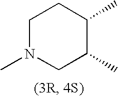 | 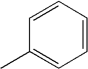 | CH₂ |
| 146 | 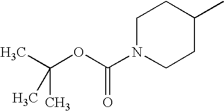 | 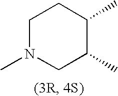 | 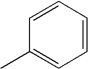 | CH₂ |
| 147 | 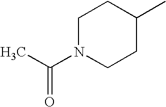 | 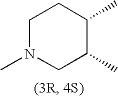 | 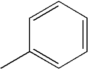 | CH₂ |

| Ex. No. | 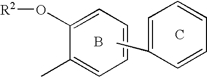 | Additives | MS (ESI) |
|---|---|---|---|
| 138 | 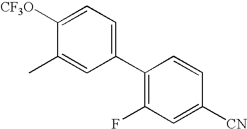 | maleic acid | 623 (M − 116 + H)⁺ |
| 139 | 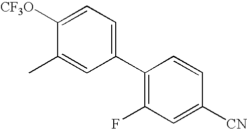 | fumaric acid | 623 (M − 116 + H)⁺ |
| 140 | 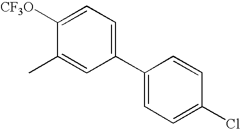 | H₂O | 600 (M + H)⁺ |
| 141 | 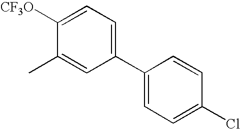 | maleic acid | 600 (M − 116 + H)⁺ |
| 142 | 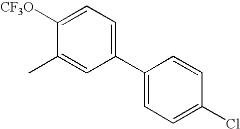 | fumaric acid | 600 (M − 116 + H)⁺ |
| 143 | 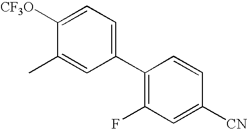 | HCl | 568 (M − HCl + H)⁺ |
| 144 | 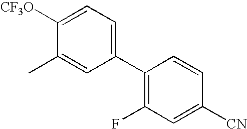 | | 570 (M + H)⁺ |
| 145 | 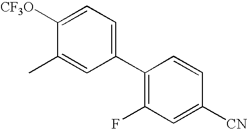 | HCl | 582 (M − HCl + H)⁺ |
| 146 | 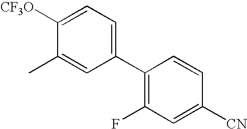 | | 653 (M + H)⁺ |
| 147 | 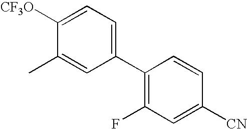 | | 595 (M + H)⁺ |

### Preparative Example 1

| | |
|---|---|
| (1) Compound of Example 1 | 10 mg |
| (2) Lactose | 60 mg |
| (3) Corn starch | 35 mg |
| (4) Hydroxypropylmethylcellulose | 3 mg |
| (5) Magnesium stearate | 2 mg |

A mixture of the compound (10 mg) obtained in Example 1, lactose (60 mg) and corn starch (35 mg) is granulated using an aqueous solution (0.03 mL) of 10 wt % hydroxypropylmethylcellulose (3 mg as hydroxypropylmethylcellulose), and then dried at 40° C. and sieved. The obtained granules are mixed with magnesium stearate (2 mg) and compressed. The obtained uncoated tablets are sugar-coated with an aqueous suspension of sucrose, titanium dioxide, talc and gum Arabic. The thus-coated tablets are glazed with bees wax to obtain finally-coated tablets.

### Preparative Example 2

| | |
|---|---|
| (1) Compound of Example 1 | 10 mg |
| (2) Lactose | 70 mg |
| (3) Corn starch | 50 mg |
| (4) Soluble starch | 7 mg |
| (5) Magnesium stearate | 3 mg |

The compound obtained in Example 1 (10 mg) and magnesium stearate (3 mg) are granulated with an aqueous soluble starch solution (0.07 mL, 7 mg as soluble starch), dried, and mixed with lactose (70 mg) and corn starch (50 mg). The mixture is compressed to obtain tablets.

### Reference Preparative Example 1

| | |
|---|---|
| (1) Rofecoxib | 5.0 mg |
| (2) Sodium chloride | 20.0 mg |
| (3) Distilled water | to 2.0 mL of total volume |

Rofecoxib (5.0 mg) and sodium chloride (20.0 mg) are dissolved in distilled water, and water is added to make the total volume 2.0 mL. The solution is filtered, and filled into ampoule (2 mL) under sterile condition. The ampoule is sterilized, and then sealed to obtain a solution for injection.

### Reference Preparative Example 2

| | | |
|---|---|---|
| (1) Rofecoxib | 50 | mg |
| (2) Lactose | 34 | mg |
| (3) Corn starch | 10.6 | mg |
| (4) Corn starch (paste) | 5 | mg |
| (5) Magnesium stearate | 0.4 | mg |
| (6) Calcium carboxymethylcellulose | 20 | mg |
| total | 120 | mg |

The above-mentioned (1) to (6) are mixed according to a conventional method and tableted by a tablet machine to obtain tablets.

### Preparative Example 3

The formulation prepared in Preparative Example 1 or 2, and the formulation prepared in Reference Preparative Example 1 or 2 are combined.

### Experimental Example 1

Radioligand receptor binding inhibitory activity (Binding inhibitory activity using receptor from human lymphoblast cells (IM-9))

The method of M. A. Cascieri et al., [Molecular Pharmacology, vol. 42, p. 458 (1992)] was modified and used. The receptor was prepared from human lymphoblast cells (IM-9). IM-9 cells (2×10⁵ cells/mL) were incubated for 3 days (one liter), which was then subjected to centrifugation for 5 minutes at 500×G to obtain cell pellets. The obtained pellets were washed once with phosphate buffer (Flow Laboratories, CAT. No. 28-103-05), which were then homogenized using Polytron homogenizer (“Kinematika”, Germany) in 30 mL of 50 mM Tris-HCl buffer (pH 7.4) containing 120 mM sodium chloride, 5 mM potassium chloride, 2 μg/mL chymostatin, 40 μg/mL bacitracin, 5 μg/mL phosphoramidon, 0.5 mM phenylmethylsulfonyl fluoride, and 1 mM ethylenediamine tetraacetate, which was subjected to centrifugation at 40,000×G for 20 minutes. The residue was washed twice with 30 mL of the above-mentioned buffer, which was then preserved frozen (−80° C.) as a specimen of the receptor.

The specimen was suspended in a reaction buffer (50 mM Tri-HCl buffer (pH 7.4), 0.02% bovine serum albumin, 1 mM phenylmethylsulfonyl fluoride, 2 μg/mL chymostatin, 40 μg/mL bacitracin and 3 mM manganese chloride) to have protein in the concentration of 0.5 mg/mL of protein and 100 μL portion of the suspension was used in the reaction. After addition of the sample and ¹²⁵I-BHSP (0.46 KBq), the reaction was allowed to proceed in 0.2 mL of reaction buffer at 25° C. for 30 minutes. The amount of nonspecific binding was determined by adding substance P at a final concentration of 2×10⁻⁶ M.

After the reaction, using a cell harvester (290 PHD, Cambridge Technology, Inc., U.S.A.), filtration was carried out through a glass filter (GF/B, Whatman, U.S.A.), which was immersed in 0.1% polyethyleneimine for 24 hours and dried. After washing three times with 250 μL of 50 mM Tris-HCl buffer (pH 7.4) containing 0.02% bovine serum albumin, the radioactivity remaining on the filter was determined with a gamma counter.

The antagonistic activity of each compound obtained in Examples was determined in terms of the concentration necessary to cause 50% inhibition (IC₅₀ value) under the above-described conditions, and the results were shown in Table (Table 16).

TABLE 16

| Example No. | IC₅₀ (nM) |
|---|---|
| 10 | 0.019 |
| 11 | 0.016 |
| 15 | 0.020 |
| 16 | 0.031 |
| 17 | 0.022 |
| 18 | 0.036 |
| 21 | 0.019 |
| 22 | 0.018 |
| 23 | 0.015 |
| 24 | 0.021 |
| 25 | 0.018 |
| 32 | 0.015 |
| 33 | 0.015 |
| 35 | 0.017 |
| 36 | 0.017 |
| 41 | 0.019 |
| 42 | 0.014 |
| 43 | 0.015 |
| 44 | 0.015 |
| 72 | 0.023 |
| 96 | 0.021 |
| 97 | 0.023 |
| 100 | 0.015 |
| 101 | 0.024 |
| 102 | 0.013 |
| 104 | 0.019 |
| 116 | 0.058 |
| 132 | 0.140 |
| 137 | 0.042 |

Radioligand means substance P labeled with [¹²⁵I].

From Table 16, it is understood that the compounds of the present invention have an excellent antagonistic action for the substance P receptors.

This application is based on a patent application No. 2005-227183 filed in Japan, and PCT/JP2006/315899 the contents of which are incorporated in full herein by this reference.

1. A compound represented by the formula: 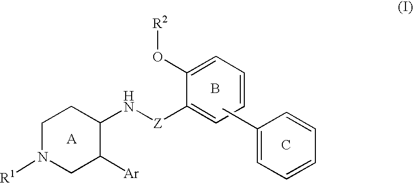
wherein Ar is a phenyl group optionally having substituent(s), R¹ is a hydrogen atom, a hydrocarbon group optionally having substituent(s), an acyl group or a heterocyclic group optionally having substituent(s), R² is a hydrogen atom, a C₁₋₆ alkyl group optionally having substituent(s) or a C₃₋₆ cycloalkyl group optionally having substituent(s), Z is a methylene group optionally having a C₁₋₆ alkyl group, ring A is a piperidine ring optionally further-having substituent(s), ring B and ring C are benzene rings optionally further having substituent(s), and R² optionally forms a ring together with the adjacent substituent on the ring B, except the compounds represented by the formula: 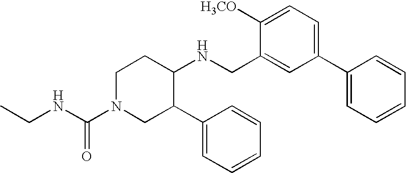
and the formula: 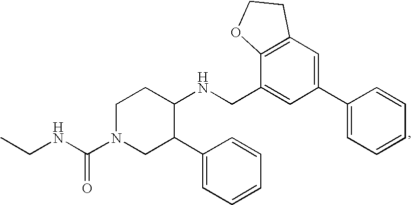
or a salt thereof.

2. The compound of claim 1, which is represented by the formula: 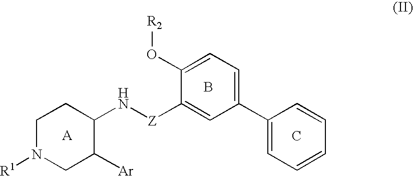
wherein the symbols in the formula are as defined in claim 1.

3. The compound of claim 1, which is represented by the formula: 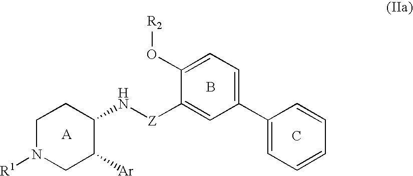
wherein the symbols in the formula are as defined in claim 1.

4. The compound of any one of claims 1 to 3, wherein R¹ is a hydrogen atom or an acyl group.

5. The compound of any one of claims 1 to 3, wherein R² is a hydrogen atom or a C₁₋₆ alkyl group optionally having substituent(s).

6. The compound of any one of claims 1 to 3, wherein Z is a methylene group optionally having a methyl group.

7. The compound of claim 3, wherein Ar is a phenyl group optionally having 1 to 3 halogen atoms;
R¹ is (1) a hydrogen atom,
(2) a C₁₋₆ alkyl-carbonyl optionally having 1 or 2 substituents selected from the group consisting of (i) an amino, (ii) a C₁₋₆ alkoxy, (iii) a C₁₋₆ alkyl-carbonylamino, (iv) a C₁₋₆ alkoxy-carbonylamino, (v) a C₁₋₆ alkylsulfonylamino, (vi) a 5- or 6-membered nitrogen-containing heterocyclic group optionally having 1 to 5 substituents selected from the group consisting of a C₁₋₆ alkyl and an oxo, said heterocyclic group optionally forms a spiro ring together with cyclopentane or cyclohexane, (vii) a C₁₋₆ alkyl-carbonyloxy, (viii) a hydroxy and (ix) a carbamoyl,
(3) a C₁₋₆ alkoxy-carbonyl,
(4) a C₁₋₆ alkylamino-carbonyl,
(5) a C₁₋₆ alkylsulfonyl,
(6) an aminocarbonylcarbonyl,
(7) a C₁₋₆ alkylamino-carbonylcarbonyl,
(8) a di-C₁₋₆ alkylamino-carbonylcarbonyl, or
(9) a piperidin-4-ylcarbonyl optionally having 1 or 2 substituents selected from the group consisting of (i) a C₁₋₆ alkyl-carbonyl optionally having a 5- or 6-membered nitrogen-containing heterocyclic group optionally having 1 or 2 oxo, (ii) a C₁₋₆ alkoxy-carbonyl, (iii) a C₁₋₆ alkylsulfonyl, (iv) a C₁₋₆ alkyl-carbonylamino-C₁₋₆ alkyl-carbonyl, (v) a di-C₁₋₆ alkyl-carbamoyl and (vi) an oxo;
R² is (1) a hydrogen atom or (2) a C₁₋₆ alkyl group optionally having 1 to 3 halogen atoms;
Z is a methylene group optionally having a methyl group;
ring A is a piperidine ring without a further substituent;
ring B is a benzene ring optionally further having a halogen atom or a C₁₋₆ alkyl or ring B forms a 2,3-dihydrobenzofuran ring together with R²; and
ring C is a benzene ring optionally having 1 or 2 substituents selected from the group consisting of
(1) a cyano,
(2) a nitro,
(3) a halogen atom,
(4) a C₁₋₆ alkyl optionally having 1 to 3 halogen atoms,
(5) a C₁₋₆ alkynyl,
(6) a C₁₋₆ alkoxy optionally having 1 to 3 halogen atoms,
(7) a C₁₋₆ alkylthio,
(8) a C₁₋₆ alkylsulfonyl,
(9) a di-C₁₋₆ alkylamino,
(10) a C₁₋₆ alkyl-carbonyl,
(11) a C₁₋₆ alkyl-carbonylamino,
(12) a C₁₋₆ alkoxy-carbonyl and
(13) a carbamoyl.

8. A compound represented by the formula: 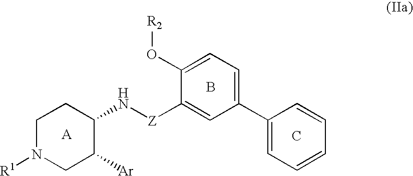
wherein
Ar is a phenyl group optionally having 1 to 3 halogen atoms;
R¹ is (1) a hydrogen atom,
(2) a C₁₋₆ alkyl-carbonyl optionally having 1 or 2 substituents selected from the group consisting of (i) an amino, (ii) a C₁₋₆ alkoxy, (iii) a C₁₋₆ alkyl-carbonylamino, (iv) a C₁₋₆ alkoxy-carbonylamino, (v) a C₁₋₆ alkylsulfonylamino, (vi) a 5- or 6-membered nitrogen-containing heterocyclic group optionally having 1 to 5 substituents selected from the group consisting of a C₁₋₆ alkyl and an oxo, said heterocyclic group optionally forms a spiro ring together with cyclopentane or cyclohexane, (vii) a C₁₋₆ alkyl-carbonyloxy, (viii) a hydroxy and (ix) a carbamoyl,
(3) a C₁₋₆ alkoxy-carbonyl,
(4) a C₁₋₆ alkylsulfonyl,
(5) an aminocarbonylcarbonyl,
(6) a C₁₋₆ alkylamino-carbonylcarbonyl,
(7) a di-C₁₋₆ alkylamino-carbonylcarbonyl, or
(8) a piperidin-4-ylcarbonyl optionally having 1 or 2 substituents selected from the group consisting of (i) a C₁₋₆ alkyl-carbonyl optionally having a 5- or 6-membered nitrogen-containing heterocyclic group optionally having 1 or 2 oxo, (ii) a C₁₋₆ alkoxy-carbonyl, (iii) a C₁₋₆ alkylsulfonyl, (iv) a C₁₋₆ alkyl-carbonylamino-C₁₋₆ alkyl-carbonyl, (v) a di-C₁₋₆ alkyl-carbamoyl and (vi) an oxo;
R² is (1) a hydrogen atom or (2) a C₁₋₆ alkyl group optionally having 1 to 3 halogen atoms;
Z is a methylene group optionally having a methyl group;
ring A is a piperidine ring without a further substituent;
ring B is a benzene ring optionally further having a halogen atom or a C₁₋₆ alkyl or ring B forms a 2,3-dihydrobenzofuran ring together with R²; and
ring C is a benzene ring optionally having 1 or 2 substituents selected from the group consisting of
(1) a cyano,
(2) a nitro,
(3) a halogen atom,
(4) a C₁₋₆ alkyl optionally having 1 to 3 halogen atoms,
(5) a C₁₋₆ alkynyl,
(6) a C₁₋₆ alkoxy optionally having 1 to 3 halogen atoms,
(7) a C₁₋₆ alkylthio,
(8) a C₁₋₆ alkylsulfonyl,
(9) a di-C₁₋₆ alkylamino,
(10) a C₁₋₆ alkyl-carbonyl,
(11) a C₁₋₆ alkyl-carbonylamino,
(12) a C₁₋₆ alkoxy-carbonyl and
(13) a carbamoyl,
or a salt thereof.

9. N-{2-[(3R,4S)-4-({[4′-chloro-4-(trifluoromethoxy)biphenyl-3-yl]methyl}amino)-3-phenylpiperidin-1-yl]-2-oxoethyl}acetamide,
3′-[({(3R,4S)-1-[(5,5-dimethyl-2,4-dioxo-1,3-oxazolidin-3-yl)acetyl]-3-phenylpiperidin-4-yl}amino)methyl]-2-fluoro-4′-(trifluoromethoxy)biphenyl-4-carbonitrile,
2-fluoro-3′-({[(3R,4S)-1-glycoloyl-3-phenylpiperidin-4-yl]amino}methyl)-4′-(trifluoromethoxy)biphenyl-4-carbonitrile,
3′-[({(3R,4S)-1-[(1-acetylpiperidin-4-yl)carbonyl]-3-phenylpiperidin-4-yl}amino)methyl]-2-fluoro-4′-(trifluoromethoxy)biphenyl-4-carbonitrile,
3′-[({(3R,4S)-1-[(2,6-dioxopiperidin-4-yl)carbonyl]-3-phenylpiperidin-4-yl}amino)methyl]-2-fluoro-4′-(trifluoromethoxy)biphenyl-4-carbonitrile,
2-[(3R,4S)-4-({[4′-cyano-2′-fluoro-4-(trifluoromethoxy)biphenyl-3-yl]methyl}amino)-3-phenylpiperidin-1-yl]-2-oxoacetamide,
3-{2-[(3R,4S)-4-({[4′-chloro-4-(trifluoromethoxy)biphenyl-3-yl]methyl}amino)-3-phenylpiperidin-1-yl]-2-oxoethyl}-5,5-dimethyl-1,3-oxazolidine-2,4-dione,
4-{[(3R,4S)-4-({[4′-chloro-2′-fluoro-4-(trifluoromethoxy)biphenyl-3-yl]methyl}amino)-3-phenylpiperidin-1-yl]carbonyl}piperidine-2,6-dione,
3′-[({(3R,4S)-1-[(5,5-dimethyl-2,4-dioxo-1,3-oxazolidin-3-yl)acetyl]-3-phenylpiperidin-4-yl}amino)methyl]-4′-(trifluoromethoxy)biphenyl-4-carbonitrile,
or a salt thereof.

10. A prodrug of the compound of claim 1.

11. A pharmaceutical agent comprising the compound of claim 1 or a prodrug thereof.

12. The pharmaceutical agent of claim 11, which is a tachykinin receptor antagonist.

13. The pharmaceutical agent of claim 11, which is an agent for the prophylaxis or treatment of lower urinary tract symptoms, a digestive organ disease or a central nerve disease.

14. The pharmaceutical agent of claim 11, which is an agent for the prophylaxis or treatment of overactive bladder, lower urinary tract symptoms associated with benign prostatic hyperplasia, pelvic visceral pain, lower urinary tract symptoms associated with chronic prostatitis, lower urinary tract symptoms associated with interstitial cystitis, irritable bowel syndrome, inflammatory bowel disease, vomiting, nausea, depression, anxiety neurosis, anxiety or sleep disorder (insomnia).

15. A method for the prophylaxis or treatment of lower urinary tract symptoms, a digestive organ disease or a central nerve disease, which comprises administering an effective amount of the compound of claim 1 or a prodrug thereof to a mammal.

16. Use of the compound of claim 1 or a prodrug thereof for the production of an agent for the prophylaxis or treatment of lower urinary tract symptoms, a digestive organ disease or a central nerve disease.
